(19) United States
(12) Patent Application Publication
(10) Pub. No.: US 2026/0123633 A1
(43) Pub. Date: May 7, 2026

(54) METHANOTROPH STRAINS FOR MITIGATING METHANE AND METHODS RELATED THERETO

(71) Applicant: NewLeaf Symbiotics, Inc., St. Louis, MO (US)

(72) Inventors: Natalie Breakfield, St. Louis, MO (US); Desmond R. Jimenez, Woodland, CA (US); David Flack, Upper Arlington, OH (US); Anthony Neumann, Manchester, MO (US)

(73) Assignee: NewLeaf Symbiotics, Inc., St. Louis, MO (US)

(21) Appl. No.: 18/992,756

(22) PCT Filed: Jul. 12, 2023
(86) PCT No.: PCT/US2023/070041

§ 371 Date: Jan. 9, 2025

Related U.S. Application Data
(60) Provisional application No. 63/388,472, filed on Jul. 12, 2022.
(60) Provisional application No. 63/383,405, filed on Nov. 11, 2022.
(60) Provisional application No. 63/489,104, filed on Mar. 8, 2023.

(51) Int. Cl. A01N 63/20; A01P 21/00; C12N 1/205; C12N 9/02; C12Q 1/02; C12R 1/01

(52) U.S. Cl. CPC A01N 63/20; A01P 21/00; C12N 1/205; C12N 9/0073; C12Q 1/02; C12Y 114/13025; C12R 2001/01

(57) ABSTRACT

Methanotroph strains that enhance early growth of plants, improve propagation/transplant vigor, increase nutrient uptake, improve stand establishment, improve stress tolerance and/or increase a plant's ability to utilize nutrients are provided herein. Uses of compostions comprising such strains and optionally methylotroph strains, for methane mitigation and crop improvement is provided. Also provided are methods to reduce green-house gas emission and convert methane to methanol with methanotroph strains.

### CROSS-REFERENCE TO RELATED APPLICATIONS

This patent application claims the benefit of U.S. Provisional Patent Application No. 63/338,472, filed Jul. 12, 2022; U.S. Provisional Patent Application No. 63/383,405, filed Nov. 11, 2022; and U.S. Provisional Patent Application No. 63/489,104, filed Mar. 8, 2023; the entire disclosure of which are incorporated herein by reference.

### INCORPORATION OF SEQUENCE LISTING XML

A computer readable form of the Sequence Listing XML containing the file named “NLSYM7007.WO Sequence Listing.xml,” which is 349,090 bytes in size (as measured in MICROSOFT WINDOWS® EXPLORER) and was created on Jul. 11, 2023, is provided herein and is herein incorporated by reference. This Sequence Listing consists of SEQ ID NOs: 1-128.

### BACKGROUND

Concentrations of atmospheric methane are rapidly increasing, and sources of methane include agricultural sources, such as rice paddies and ruminants, wetlands, landfills, waste facilities, animal feed, and water. Biologic methods to reduce or mitigate methane from such sources are desired. In addition, methods of enhancing plant production by improving growth and/or increasing nutrient utilization by biologic methods are desired.

One-carbon organic compounds such as methane and methanol are found extensively in nature, and may be utilized as carbon sources by a diverse group of bacteria. Methanotrophs possess the enzyme methane monooxygenase (MMO) which incorporates an atom of oxygen from O₂ into methane, forming methanol. There are two forms of MMO, a soluble methane monooxygenase (sMMO), and a particulate methane monooxygenase (pMMO). Most known methanotrophs possess pMMO, and sMMO is also present in some methanotrophs. Methanotrophs are classified into three groups, Type I, Type II and Type X on the basis of various physiological and morphological differences. Type I and Type X methanotrophs are gammaproteobacteria, while Type II methanotrophs are alphaproteobacterial. Some methanotrophs have been reported to contain two distinct isozymes of particulate methane monooxygenase (pMMO). pMMO1 facilitates oxidation of methane under conditions where methane is at high concentrations, and pMMO2 facilitates oxidation of methane in environments where the methane concentration is low, including oxidation of atmospheric methane. pMMO consists of three protein subunits, PmoA, PmoB and PmoC, which are encoded on an operon present in the methanotroph genome.

Methylotrophs, as defined herein, can utilize more complex organic compounds, such as organic acids, higher alcohols, sugars, and the like. Thus, methylotrophic bacteria can be facultative methylotrophs. Some methylotrophic bacteria in the Methylobacterium or Methylorubrum genera are pink-pigmented. They are conventionally referred to as PPFM bacteria, being pink-pigmented facultative methylotrophs.

All nations have tried to frame a global regime to control green-house gas emissions and to assist with adaptation and yet emissions have continued to increase. Methane is a critical component of Earth's carbon cycle and contributes to global warming. Agriculture (e.g., enteric fermentation in livestock, manure management, and rice cultivation) is a contributor to global CH₄ emission. Implementation of a biological methane oxidizing technology has the potential for mitigation of atmospheric methane levels and reduction of green-house gas emissions.

### SUMMARY

Provided herein are strains of methanotrophic bacteria and compositions comprising one or more methanotroph strains, wherein the strains are capable of using methane as a carbon source for growth. Also provided are methods that utilize such strains for mitigating methane production, for example in agricultural applications, including plant production in flooded fields, for reducing methane produced in animal production, such as cattle or dairy industries, or for reducing natural methane sources that exist in wetlands or other natural water sources, (including but not limited to lakes, rivers, mangroves, marshes, bogs and streams), in geological sources, or in gases produced as the result of wildfires, wild animals, or insects. In some embodiments provided herein methanotrophs are applied to rice plants resulting in decreased levels of methane and enhanced plant growth.

In some embodiments, methanotroph strains in the methods and compositions provided herein are species from a bacterial genus selected from the group consisting of Methyloacidimicrobium, Methyloacidiplilum, Methylobacter, Methylocaldum, Methylocapsa, Methylocella, Methylococcus, Methylocystis, Methyloferula, Methylogaea, Methyloglobus, Methylohalobius, Methylomagnum, Methylomarinum, Methylomicrobium, Methylomonas, Methyloparacoccus, Methyloperedens, Methyloprofundus, Methylosarcina, Methylosinus, Methylosoma, Methylosphaera, Methylothermus, and Methylovulum. In some embodiments, a methanotroph provided herein is a Methylocystis species selected from M. hirsuta, M. rosea and M. parvus.

In some embodiments, methanotroph strains in the methods and compositions provided herein are Type II (Alphaproteobacteria) strains that comprise a pMMO2 methane monooxygenase encoded by an operon comprising expression sequences for pMMO2 protein components PmoA2, PmoB2 and PmoC2. In some embodiments, the Type II methanotroph is a Methylocystis species. In some embodiments a methanotroph provided herein is a Methylocystis hirsuta isolate and PmoA2, PmoB2 and PmoC2 have protein sequences of SEQ ID NOS: 76-78 or SEQ ID NOS: 79-81. In some embodiments, a Methylocystis hirsuta strain provided herein comprises a pMMO2 monooxygenase having PmoA2, PmoB2 and PmoC2 proteins with sequences at least 97, 98 or 99% identical to SEQ ID NOS:76-78 or SEQ ID NOS: 79-81. In some embodiments, a Methylocystis hirsuta strain is selected from the group consisting of NLS1505, NLS1506, NLS1508, NLS1509, NLS1511, and NLS1512, and variants thereof. In some embodiments, Methylocystis hirsuta bacterial strains provided herein comprise sMMO proteins in addition to pMMO proteins.

In some embodiments, methanotroph strains in the methods and compositions provided herein are Type I (Gammaproteobacter) strains. In some embodiments, Type I methanotrophs are species of Methylomicrobium or Methylosarcina. In some embodiments, a Methylomicrobium isolate comprises a PmoA protein at least 97, 98 or 99% identical to SEQ ID NO:83 or SEQ ID NO:84. In some embodiments, a Methylomicrobium isolate comprises a PmoB protein at least 97, 98 or 99% identical to SEQ ID NO:85 or SEQ ID NO:86. In some embodiments, a Methylomicrobium isolate comprises a PmoC protein at least 97, 98 or 99% identical to SEQ ID NO:87 or SEQ ID NO:88. In some embodiments, a Methylosarcina isolate comprises a PmoA protein at least 97, 98 or 99% identical to SEQ ID NO:89, SEQ ID NO:90 or SEQ ID NO:91. In some embodiments, a Methylosarcina isolate comprises a PmoB protein at least 97, 98 or 99% identical to SEQ ID NO:92 or SEQ ID NO:93. In some embodiments, a Methylosarcina isolate comprises a PmoC protein at least 97, 98 or 99% identical to SEQ ID NO:94, SEQ ID NO:95 or SEQ ID NO:96. In some embodiments the methanotrophs are isolates of Methylomicrobium lacus or Methylosarcina fibrata. In some embodiments, a Methylomicrobium lacus isolate is NLS1501. In some embodiments a Methylosarcina fibrata isolate is NLS1504. In some embodiments, methanotroph bacterial strains provided herein comprise sMMO proteins in addition to pMMO proteins. In some embodiments, a methanotroph strain for use in the compositions and methods provided herein comprises a 16S encoding sequence of any one of SEQ ID NO:118-120.

Also provided herein are methods of using methanotroph strains and compositions to improve plant growth resulting in increased biomass and yield. In some embodiments, a methanotroph that improves plant growth and yield is selected from the group consisting of a Methylocystis species, a Methylomicrobium species and a Methylosarcina species. In some embodiments, a methanotroph that enhances plant growth and yield is selected from the group consisting of NLS1501, NLS1504 and NLS1508. In some embodiments, methanotrophs improve plant growth and production by fixing nitrogen as the result of the presence and expression of core nif genes in the methanotroph genome. In some embodiments, the increased availability of nitrogen to the plant results in growth promotion and increased plant size and yield and provides for enhanced growth even when limiting levels of nitrogen fertilizer are applied to the plant. In some embodiments, a methanotroph that improves plant production by fixing nitrogen is selected from the group consisting of NLS1501, NLS1504, NLS1505, NLS1506, NLS1508, NLS1509, NLS1511, and NLS1512.

In further embodiments, a method and composition provided herein comprises one or more methanotroph strains and one or more methylotrophic bacterial strains. In some embodiments, a provided methyltroph enhances plant growth and/or provides for one or more plant production benefits, for example but not by way of limitation, enhanced nitrogen uptake and or utilization, enhanced early growth of plants, improved yield of a crop and/or crop product, improved propagation/transplant vigor, increased nutrient uptake, improved stand establishment, improved stress tolerance, improved pest resistance, improved pathogen resistance, fruit ripening and/or increased ability of the plant to utilize nutrients, such as nitrogen, potassium, sulfur, cobalt, copper, zinc, phosphorus, boron, iron, and manganese. In some embodiments, a methylotroph in the methods and compositions provided herein is capable of oxidizing methane. In some embodiments, a methylotroph in compositions and methods provided herein is a Methylobacterium or Methylorubrum species.

In some embodiments, compositions for application to plants, plant parts, a plant growth environment or other methane source, comprise one or more methanotrophs and one or more methylotrophs. In some embodiments, methods are provided wherein methanotrophs and methylotrophs are applied to plants, plant parts, a plant growth environment, or other methane sources, in separate compositions. In some embodiments, methanotrophs and/or methylotrophs are applied at multiple times, for example to plants, plant parts, or a plant growth environment at various stages of plant growth. In some embodiments, methanotrophs and methylotrophs are applied in the same composition. In some embodiments, a methanotroph employed in such compositions and methods is a Methylocystis species, a Methylomicrobium species or a Methylosarcina species. In some embodiments, a methanotroph strain for use in the compositions and methods provided herein comprises a 16S encoding sequence of any one of SEQ ID NO:118-120. In some embodiments, a methylotroph employed in such compositions and methods is a Methylobacterium or Methylorubrum species capable of oxidizing methane and/or facilitating growth and function of the applied methanotroph strain or strains. In some embodiments, a methylotroph is a strain disclosed in Table 1 herein. In some embodiment, a methytroph is a species selected from the group consisting of M. radiotolerans, M. populi and M. extorquens. In some embodiments, a methylotroph strain in the methods and compositions provided herein is selected from the group consisting of LGP2002 (NRRL B-50931), LGP2003 (NRRL B-50932), LGP2004 (NRRL B-50933), LGP2009 (NRRL B-50938), LGP2015 (NRRL B-67340), LGP2016 (NRRL B-67341), LGP2017 (NRRL B-67741), LGP2019 (NRRL B-67743), NLS0693 (NRRL B-67926), LGP2020 (NRRL-B-67892), and NLS7725.

In some embodiments, a methylotroph strain for use in the compositions and methods provided herein comprises a 16S encoding sequence of SEQ ID NO: 121-122. In some embodiments the methyltroph is M. radiotolerans strain LGP2020 (NRRL-B-67892) or M. populi strain NLS7725. In some embodiments, a methanotroph strain is selected from the group consisting of NLS1501, NLS1504, NLS1505, NLS1506, NLS1508, NLS1509, NLS1511, and NLS1512; and a methylotroph strain is selected from the group consisting of LGP2000 (NRRL B-50929), LGP2001 (NRRL B-50930), LGP2002 (NRRL B-50931), LGP2003 (NRRL B-50932), LGP2004 (NRRL B-50933), LGP2005 (NRRL B-50934), LGP2006 (NRRL B-50935), LGP2007 (NRRL B-50936), LGP2008 (NRRL B-50937), LGP2009 (NRRL B-50938), LGP2010 (NRRL B-50939), LGP2011 (NRRL B-50940), LGP2012 (NRRL B-50941), LGP2013 (NRRL B-50942), LGP2014 (NRRL B-67339), LGP2015 (NRRL B-67340), LGP2016 (NRRL B-67341), LGP2017 (NRRL B-67741), LGP2018 (NRRL B-67742), LGP2019 (NRRL B-67743), NLS0497 (NRRL B-67925), NLS0693 (NRRL B-67926), NLS1179 (NRRL B-67929), LGP2167 (NRRL B-67927), LGP2020 (NRRL-B-67892), LGP2021 (NRRL-B-68032), LGP2022 (NRRL-B-68033), LGP2023 (NRRL-B-68034), LGP2028 (NRRL B-68064), LGP2029 (NRRL B-68065), LGP2030 (NRRL B-68066), LGP2031 (NRRL B-68067), LGP2033 (NRRL B-68068), LGP2034 (NRRL B-68069), NLS0665 (NRRL B-68194), NLS0754 (NRRL B-68197), NLS0672 (NRRL B-68196), NLS0729 (NRRL B-68195), NLS0049 (NRRL-B-68236), NLS0591 (NRRL-B-68215), NLS0439 (NRRL-B-68216), NLS1310 (NRRL-B-68217), NLS1312 (NRRL-B-68218), NLS0612 (NRRL-B-68237), NLS0706 (NRRL-B-68238), NLS0725 (NRRL-B-68239), NLS0770 (NRRL-B-68075), NLS0737 (NRRL-B-68074), NLS5278 (NRRL-B-68186), NLS5334 (NRRL-B-68187), NLS5480 (NRRL-B-68188), NLS5549 (NRRL-B-68189), NLS7725, and variants thereof. In certain embodiments, a variant of an additional Methylobacterium is identified by the presence of one or more of SEQ ID NOs: 33-75. In some embodiments, a methanotroph strain is NLS1501, NLS1504 or NLS1508 and a methylotroph strain is LGP2019 (NRRL B-67743), LGP2020 (NRRL-B-67892) or NLS7725. In some embodiment, a methytroph is a species selected from the group consisting of M. radiotolerans, M. populi and M. extorquens. In some embodiments, a methylotroph strain in the methods and compositions provided herein is selected from the group consisting of LGP2002 (NRRL B-50931), LGP2003 (NRRL B-50932), LGP2004 (NRRL B-50933), LGP2009 (NRRL B-50938), LGP2015 (NRRL B-67340), LGP2016 (NRRL B-67341), LGP2017 (NRRL B-67741), LGP2019 (NRRL B-67743), NLS0693 (NRRL B-67926), LGP2020 (NRRL-B-67892), and NLS7725. In some embodiments, a methanotroph is NLS1508 and a methylotroph is LGP2019 (NRRL B-67743). In some embodiments, a methanotroph is NLS1501 and a methylotroph is NLS7725. In some embodiments, a methanotroph is NLS1501 and a methylotroph is LGP2020 (NRRL-B-67892).

In some embodiments, a methanotroph is NLS1508 and a methylotroph is LGP2019 (NRRL B-67743), in some embodiments, a methanotroph is NLS1508 and a methylotroph is LGP2020 (NRRL-B-67892), in some embodiments, a methanotroph is NLS1508 and a methylotroph is NLS7725. In some embodiments, a methanotroph is NLS1501 and a methylotroph is LGP2019 (NRRL B-67743), in some embodiments, a methanotroph is NLS1501 and a methylotroph is LGP2020 (NRRL-B-67892), in some embodiments, a methanotroph is NLS1501 and a methylotroph is NLS7725. In some embodiments, a methanotroph is NLS1504 and a methylotroph is LGP2019 (NRRL B-67743), in some embodiments, a methanotroph is NLS1504 and a methylotroph is LGP2020 (NRRL-B-67892), in some embodiments, a methanotroph is NLS1504 and a methylotroph is NLS7725. In some embodiments, a methanotroph is NLS1501 and a methylotroph is NLS7725. In some embodiments, a methanotroph is NLS1501 and a methylotroph is LGP2020 (NRRL-B-67892). In some embodiments, a methanotroph is NLS1508 and a methylotroph is LGP2019 (NRRL B-67743). In some embodiments, a methanotroph is NLS1501 and a methylotroph is NLS7725. In some embodiments, a methanotroph is NLS1501 and a methylotroph is LGP2020 (NRRL-B-67892).

In some embodiments, the compositions provided herein comprise microbial strains providing additional plant benefits. In certain embodiments, microbial strains in the compositions that provide additional plant benefits are Methylobacterium strains. In some embodiments, compositions comprising a methanotroph strain comprise one or more Methylobacterium strains that provide for at least one plant benefit selected from the group consisting of enhanced nitrogen uptake and or utilization, enhanced early growth of plants, improved yield of a crop and/or crop product, improved propagation/transplant vigor, increased nutrient uptake, improved stand establishment, improved stress tolerance, improved pest resistance, improved pathogen resistance, fruit ripening and/or increased ability of the plant to utilize nutrients, such as nitrogen, potassium, sulfur, cobalt, copper, zinc, phosphorus, boron, iron, and manganese. In some embodiments, such compositions comprise a Methylobacterium that facilitates nitrogen fixation. In certain embodiments, the compositions provided herein enhance uptake and/or utilization of one or more nutrients and/or enhances nitrogen use efficiency and/or nitrogen fixation of a treated plant or a plant grown in treated soil, and a Methylobacterium in the composition is selected from the group consisting of LGP2001 (NRRL B-50930), LGP2002 (NRRL B-50931), LGP2009 (NRRL B-50938), LGP2015 (NRRL B-67340), LGP2016 (NRRL B-67341), LGP2017 (NRRL B-67741), LGP2018 (NRRL B-67742), LGP2019 (NRRL B-67743), NLS0693 (NRRL B-67926), LGP2167 (NRRL B-67927), LGP2020 (NRRL-B-67892), LGP2021 (NRRL-B-68032), LGP2022 (NRRL-B-68033), LGP2023 (NRRL-B-68034), LGP2029 (NRRL B-68065), LGP2030 (NRRL B-68066), LGP2031 (NRRL B-68067), LGP2033 (NRRL B-68068), LGP2034 (NRRL B-68069), NLS0665 (NRRL B-68194), NLS0754 (NRRL B-68197), NLS0672 (NRRL B-68196), NLS0729 (NRRL B-68195), NLS0049 (NRRL-B-68236), NLS0591 (NRRL-B-68215), NLS0439 (NRRL-B-68216), NLS1310 (NRRL-B-68217), NLS1312 (NRRL-B-68218), NLS0612 (NRRL-B-68237), NLS0706 (NRRL-B-68238), NLS0725 (NRRL-B-68239), NLS7725, and variants thereof. In certain embodiments, a Methylobacterium or variant thereof that increases uptake and/or utilization of one or more nutrients and/or enhances nitrogen use efficiency is identified by the presence of one or more of SEQ ID NOs: 33-39, 46-60, 64-66, or 70-75. In certain embodiments, the Methylobacterium in the compositions provided herein comprise one or more genetic elements associated with the ability to enhance early plant growth, wherein the one or more genetic elements (i) is recD2_2 or pinR; or (ii) the one or more genetic elements encode a protein having a consensus amino acid sequence of SEQ ID NO: 17 to SEQ ID NO: 23. In some embodiments, Methylobacterium in the compositions provided herein that improve early plant growth also impart one or more additional beneficial traits to treated plants or plants grown from treated plant parts or seeds, wherein the trait is enhanced uptake of nutrients, enhanced assimilation of nutrients, and/or enhanced nutrient use efficiency. In some embodiments, plants treated with Methylobacterium isolates provided herein demonstrate enhanced nitrogen use efficiency. In certain embodiments, the compositions provided herein enhance yield of a treated crop or crop product of a treated crop plant or a crop plant grown in treated soil.

In some embodiments a Methylobacterium strain provided in combination with a methanotroph strain is capable of mitigating methane and comprises a soluble methane monooxygenase (sMMO) encoded by genetic elements in the genome of the Methylobacterium strain. In some embodiments, sMMO encoding genetic elements are present on a plasmid in a methane mitigating Methylobacterium strain. In some embodiments, a Methylobacterium strain capable of mitigating methane comprises genetic elements encoding SEQ ID NOS: 1-4. In some embodiments, a Methylobacterium strain capable of mitigating methane comprises genetic elements encoding SEQ ID NOS: 5-8. In some embodiments, a Methylobacterium strain capable of mitigating methane comprises genetic elements encoding sMMO components having at least 65% identity to SEQ ID NOS: 1-4 or 5-8. In some embodiments, the genetic elements comprise SEQ ID NOS: 9-12. In some embodiments, the genetic elements comprise SEQ ID NOS:13-16. In some embodiments, the genetic elements have at least 65% identity to SEQ ID NOS: 9-12 or 13-16. In some embodiments, a Methylobacterium strain capable of mitigating methane is selected from the group consisting of NLS0770 (NRRL B-68075), NLS0737 (NRRL B-68074), NLS5278 (NRRL-B-68186), NLS5334 (NRRL-B-68187), NLS5480 (NRRL-B-68188), NLS5549 (NRRL-B-68189) and variants thereof. In certain embodiments, a Methylobacterium capable of mitigating methane also provides for at least one plant benefit selected from the group consisting of enhanced early growth of plants, improved yield, improved propagation/transplant vigor, increased nutrient uptake, improved stand establishment, improved stress tolerance, and/or increased ability of the plant to utilize nutrients, such as nitrogen, potassium, sulfur, cobalt, copper, zinc, phosphorus, boron, iron, and manganese. In some embodiments, a Methylobacterium capable of mitigating methane fixates nitrogen. In certain embodiments, a Methylobacterium provided in combination with a methanotroph strain provides for at least one plant benefit selected from the group consisting of enhanced early growth of plants, improved yield, improved propagation/transplant vigor, increased nutrient uptake, improved stand establishment, improved stress tolerance, and/or increased ability of the plant to utilize nutrients, such as nitrogen, potassium, sulfur, cobalt, copper, zinc, phosphorus, boron, iron, and manganese. In some embodiments, a Methylobacterium provided in combination with a methanotroph strain fixates nitrogen.

In certain embodiments, the compositions provided herein enhance yield of a treated crop or crop product of a treated crop plant or a crop plant grown in treated soil. In some embodiments, a treated crop plant is a corn plant. In some embodiments a corn plant is treated with methanotroph NLS1508 and optionally with methylotroph LGP2019 (NRRL B-67743). In certain embodiments, the crop is rice. In some embodiments, compositions for treating a rice crop comprise a methanotroph and one or more Methylobacterium strains. In some embodiments, a methanotroph in such compositions is NLS1501, NLS1504 or NLS1508. In some embodiments a Methylobacterium in such compositions increases rice yield. In some embodiments, a Methylobacterium providing for increased rice yield is selected from the group consisting of LGP2016 (NRRL B-67341), LGP2017 (NRRL B-67741), LGP2019 (NRRL B-67743), LGP2020 (NRRL B-67892), and variants thereof. In some embodiments, a methanotroph is selected from the group consisting of NLS1501, NLS1504, NLS1505, NLS1506, NLS1508, NLS1509, NLS1511, and NLS1512, and variants thereof.

In some embodiments, methanotroph compositions provided herein further comprise a Methylobacterium capable of mitigating methane, and the Methylobacterium is selected from the group consisting of NLS0770 (NRRL-B-68075), NLS5278 (NRRL-B-68186), NLS5334 (NRRL-B-68187), NLS5480 (NRRL-B-68188), NLS5549 (NRRL-B-68189), and variants thereof. Variants of NLS0737 or NLS0770 can be identified, for example, by the presence of SEQ ID NO:31 in the genome of a methane mitigating Methylobacterium. Variants of NLS5278, NLS5334, NLS5480, or NLS5549 can be identified, for example, by the presence of SEQ ID NO:32 in the genome of a methane mitigating Methylobacterium. In some embodiments, the compositions comprise an additional Methylobacterium providing for enhanced yield of rice.

Also provided are isolated methanotroph strains selected from NLS1501, NLS1504, NLS1505, NLS1506, NLS1508, NLS1509, NLS1511, and NLS1512, and variants thereof, compositions comprising such Methylobacterium isolates or variants thereof, and plants, plant parts, or seeds that are at least partially coated with compositions comprising NLS1501, NLS1504, NLS1505, NLS1506, NLS1508, NLS1509, NLS1511, and NLS1512, and variants thereof. Variants of NLS1501, NLS1504, NLS1505, NLS1506, NLS1508, NLS1509, NLS1511, and NLS1512, can be identified, for example, by the presence of SEQ ID NO: SEQ ID NOS:97-117 in the genome of a methanotroph. In some embodiments, the plant is rice. In some embodiments, the plant is a crop grown for feeding livestock, for example grasses in a pasture where livestock feed. In some embodiments, the coated plant or plant part comprises plant material harvested for livestock feed, wherein the methanotroph is applied to a seed or to a growing a plant, and wherein harvested plant material comprises methane mitigating methanotroph. In some embodiments, a methanotroph is added directly to livestock feed.

Also provided are isolated Methylobacterium NLS7725 and variants thereof, compositions comprising such Methylobacterium isolate or variants thereof, and plants, plant parts, or seeds that are at least partially coated with compositions comprising NLS7725 or variants thereof.

Compositions comprising a fermentation product comprising a methanotroph strain, and optionally a methylotroph strain, that is essentially free of contaminating microorganisms, and methods of use of such compositions to mitigate methane and enhance plant production are provided herein. In certain embodiments, a methanotroph strain is selected from the group consisting of NLS1501, NLS1504, NLS1505, NLS1506, NLS1508, NLS1509, NLS1511, and NLS1512, and variants thereof. In certain embodiments, a composition comprises a methanotroph strain and one or more additional components including one or more agriculturally acceptable adjuvants or excipients, and/or an additional active component, for example a pesticide or a second biological. In certain embodiments, the pesticide can be, for example, an insecticide, a fungicide, an herbicide, or a nematicide. In certain embodiments, the second biological can be a strain that improves yield or controls an insect, pest, fungi, weed, or nematode. In some embodiments, a second biological is a Methylobacterium strain. In some embodiments, a composition comprises at least one methanotroph strain and one or more methylotroph strains. In some embodiments, the methylotroph strains are Methylobacterium strains. In some embodiments, a Methylobacterium strain in the compositions provided herein comprises an sMMO protein and further contributes to methane mitigation. In some embodiments, a Methylobacterium strain in the compositions provided herein provides a plant benefit selected from the group consisting of enhanced early growth of plants, improved yield, improved propagation/transplant vigor, increased nutrient uptake, improved stand establishment, improved stress tolerance, and/or increased ability of the plant to utilize nutrients, such as nitrogen, potassium, sulfur, cobalt, copper, zinc, phosphorus, boron, iron, and manganese.

Also provided herein are plants, plant parts or seeds that are treated with methanotroph and/or methylotroph strains, including for example, Methylobacterium strains, and compositions comprising such strains. Such plants can be without limitation, agricultural crop plants, fruits and vegetables, leafy green plants, herbs, ornamentals, turf grasses, golf grass, shrubs, and trees.

Methods of mitigating methane using compositions comprising methanotroph and optionally methylotroph strains are provided herein. Methane mitigation methods provided herein include methods to decrease methane levels by reducing methane emissions or by enhancing removal of methane from sources of the gas, such as agricultural soil, wetlands, landfills, waste facilities, animal feed, water or air. In one embodiment, a method for mitigating methane gas in an agricultural field comprises applying a composition to a field, plant, plant part or seed, wherein the composition comprises at least one methanotroph selected from the group consisting of NLS1501, NLS1504, NLS1505, NLS1506, NLS1508, NLS1509, NLS1511, and NLS1512. In some embodiments, a composition applied in such methods further comprises a Methylobacterium provided in Table 1 herein. In some embodiments, a Methylobacterium strain is capable of mitigating methane. In some embodiments, a Methylobacterium capable of mitigating methane is selected from the group consisting of NLS0737, NLS0770, NLS5278, NLS5334, NLS5480, NLS5549, and variants thereof. Variants of NLS0737 and NLS0770 can be identified, for example, by the presence of SEQ ID NO:31 in the genome of a methane mitigating Methylobacterium. Variants of NLS5278, NLS5334, NLS5480, or NLS5549 can be identified, for example, by the presence of SEQ ID NO:32 in the genome of a methane mitigating Methylobacterium. In such embodiments, growth of a methanotroph and optionally, a Methylobacterium, results in utilization of methane as a carbon source. In this manner, methane is oxidized and methane emissions from an agricultural field or other methane source are reduced. In some embodiments, the composition comprising a methanotroph, and optionally a Methylobacterium, is applied to an irrigated field, a flooded field, or a field that will be irrigated or will become flooded. In some embodiments, the composition comprising a methanotroph, and optionally a Methylobacterium, is applied to a rice plant, plant part or seed. In some embodiments, the composition comprising a methanotroph, and optionally a Methylobacterium, is applied to a flooded or irrigated rice field. In some embodiments, a methanotroph strain is NLS1501, NLS1504 or NLS1508 and a methylotroph strain is LGP2020 (NRRL-B-67892) or NLS7725. In some embodiments, a methanotroph is NLS1501 and a methylotroph is NLS7725. In some embodiments, a methanotroph is NLS1508 and a methylotroph is LGP2019 (NRRL B-67743). In some embodiment, a methytroph is a species selected from the group consisting of M. radiotolerans, M. aminovorans and M. extorquens. In some embodiments, a methylotroph strain in the methods and compositions provided herein is selected from the group consisting of LGP2002 (NRRL B-50931), LGP2003 (NRRL B-50932), LGP2004 (NRRL B-50933), LGP2009 (NRRL B-50938), LGP2015 (NRRL B-67340), LGP2016 (NRRL B-67341), LGP2017 (NRRL B-67741), LGP2019 (NRRL B-67743), NLS0693 (NRRL B-67926), LGP2020 (NRRL-B-67892), and NLS7725.

In some embodiments, a method for mitigating methane comprises treating a pasture, wasteland, a landfill or waste with a composition comprising at least one methanotroph, and optionally, at least one methylotroph strain or isolate; and growing the methanotroph, and optionally, the methylotroph, in the pasture, wasteland, a landfill or waste thereby mitigating methane. In some embodiments, the methanotroph is selected from the group consisting of NLS1501, NLS1504, NLS1505, NLS1506, NLS1508, NLS1509, NLS1511, NLS1512, and variants thereof, and the methylotroph is a Methylobacterium selected from the group consisting of LGP2001 (NRRL B-50930), LGP2002 (NRRL B-50931), LGP2009 (NRRL B-50938), LGP2015 (NRRL B-67340), LGP2016 (NRRL B-67341), LGP2017 (NRRL B-67741), LGP2018 (NRRL B-67742), LGP2019 (NRRL B-67743), NLS0693 (NRRL B-67926), LGP2167 (NRRL B-67927), LGP2020 (NRRL-B-67892), LGP2021 (NRRL-B-68032), LGP2022 (NRRL-B-68033), LGP2023 (NRRL-B-68034), LGP2029 (NRRL B-68065), LGP2030 (NRRL B-68066), LGP2031 (NRRL B-68067), LGP2033 (NRRL B-68068), LGP2034 (NRRL B-68069), NLS0665 (NRRL B-68194), NLS0754 (NRRL B-68197), NLS0672 (NRRL B-68196), NLS0729 (NRRL B-68195), NLS0049 (NRRL-B-68236), NLS0591 (NRRL-B-68215), NLS0439 (NRRL-B-68216), NLS1310 (NRRL-B-68217), NLS1312 (NRRL-B-68218), NLS0612 (NRRL-B-68237), NLS0706 (NRRL-B-68238), NLS0725 (NRRL-B-68239), NLS7725, NLS0737, NLS0770, NLS5278, NLS5334, NLS5480, NLS5549, and variants thereof.

In some embodiments, a methanotroph strain is NLS1501, NLS1504 or NLS1508 and a methylotroph strain is LGP2020 (NRRL-B-67892) or NLS7725.

In some embodiments, a methytroph is a species selected from the group consisting of M. radiotolerans, M. populi and M. extorquens. In some embodiments, a methylotroph strain in the methods and compositions provided herein is selected from the group consisting of LGP2002 (NRRL B-50931), LGP2003 (NRRL B-50932), LGP2004 (NRRL B-50933), LGP2009 (NRRL B-50938), LGP2015 (NRRL B-67340), LGP2016 (NRRL B-67341), LGP2017 (NRRL B-67741), LGP2019 (NRRL B-67743), NLS0693 (NRRL B-67926), LGP2020 (NRRL-B-67892), and NLS7725. In some embodiments, a methanotroph is NLS1501 and a methylotroph is NLS7725. In some embodiments, a methanotroph is NLS1508 and a methylotroph is LGP2019 (NRRL B-67743).

In some embodiments, a method for mitigating methane comprises mitigation of methane production by livestock, wherein said method comprises treating land where livestock feed or will feed, with at least one methanotroph thereby reducing methane from livestock feed. In some embodiments, the methanotroph is selected from the group consisting of NLS1501, NLS1504, NLS1505, NLS1506, NLS1508, NLS1509, NLS1511, and NLS1512. In some embodiments, where treatment further comprises a methylotroph. In some embodiments, the methylotroph is a Methylobacterium selected from the group consisting of LGP2001 (NRRL B-50930), LGP2002 (NRRL B-50931), LGP2009 (NRRL B-50938), LGP2015 (NRRL B-67340), LGP2016 (NRRL B-67341), LGP2017 (NRRL B-67741), LGP2018 (NRRL B-67742), LGP2019 (NRRL B-67743), NLS0693 (NRRL B-67926), LGP2167 (NRRL B-67927), LGP2020 (NRRL-B-67892), LGP2021 (NRRL-B-68032), LGP2022 (NRRL-B-68033), LGP2023 (NRRL-B-68034), LGP2029 (NRRL B-68065), LGP2030 (NRRL B-68066), LGP2031 (NRRL B-68067), LGP2033 (NRRL B-68068), LGP2034 (NRRL B-68069), NLS0665 (NRRL B-68194), NLS0754 (NRRL B-68197), NLS0672 (NRRL B-68196), NLS0729 (NRRL B-68195), NLS0049 (NRRL-B-68236), NLS0591 (NRRL-B-68215), NLS0439 (NRRL-B-68216), NLS1310 (NRRL-B-68217), NLS1312 (NRRL-B-68218), NLS0612 (NRRL-B-68237), NLS0706 (NRRL-B-68238), NLS0725 (NRRL-B-68239), NLS7725, NLS0737, NLS0770, NLS5278, NLS5334, NLS5480, NLS5549, and variants thereof. In some embodiments, a methanotroph strain is NLS1501, NLS1504 or NLS1508 and a methylotroph strain is LGP2020 (NRRL-B-67892) or NLS7725. In some embodiment, a methytroph is a species selected from the group consisting of M. radiotolerans, M. populi and M. extorquens. In some embodiments, a methylotroph strain in the methods and compositions provided herein is selected from the group consisting of LGP2002 (NRRL B-50931), LGP2003 (NRRL B-50932), LGP2004 (NRRL B-50933), LGP2009 (NRRL B-50938), LGP2015 (NRRL B-67340), LGP2016 (NRRL B-67341), LGP2017 (NRRL B-67741), LGP2019 (NRRL B-67743), NLS0693 (NRRL B-67926), LGP2020 (NRRL-B-67892), and NLS7725. In some embodiments, a methanotroph is NLS1501 and a methylotroph is NLS7725. In some embodiments, a methanotroph is NLS1508 and a methylotroph is LGP2019 (NRRL B-67743).

Methods for reducing methane emissions from a methane emitting source are also disclosed. These methods comprise applying a composition comprising at least one methanotroph and optionally, a methylotroph, to the methane emitting source. In some embodiments, the methylotroph is a Methylobacterium strain capable of oxidizing methane selected from the group consisting of NLS0737, NLS0770, NLS5278, NLS5334, NLS5480, NLS5549, and variants thereof. In some embodiments, the methanotroph is selected from the group consisting of NLS1501, NLS1504, NLS1505, NLS1506, NLS1508, NLS1509, NLS1511, and NLS1512, and variants thereof. In some embodiments, the methylotroph is a Methylobacterium selected from the group consisting of LGP2001 (NRRL B-50930), LGP2002 (NRRL B-50931), LGP2009 (NRRL B-50938), LGP2015 (NRRL B-67340), LGP2016 (NRRL B-67341), LGP2017 (NRRL B-67741), LGP2018 (NRRL B-67742), LGP2019 (NRRL B-67743), NLS0693 (NRRL B-67926), LGP2167 (NRRL B-67927), LGP2020 (NRRL-B-67892), LGP2021 (NRRL-B-68032), LGP2022 (NRRL-B-68033), LGP2023 (NRRL-B-68034), LGP2029 (NRRL B-68065), LGP2030 (NRRL B-68066), LGP2031 (NRRL B-68067), LGP2033 (NRRL B-68068), LGP2034 (NRRL B-68069), NLS0665 (NRRL B-68194), NLS0754 (NRRL B-68197), NLS0672 (NRRL B-68196), NLS0729 (NRRL B-68195), NLS0049 (NRRL-B-68236), NLS0591 (NRRL-B-68215), NLS0439 (NRRL-B-68216), NLS1310 (NRRL-B-68217), NLS1312 (NRRL-B-68218), NLS0612 (NRRL-B-68237), NLS0706 (NRRL B-68238), NLS0725 (NRRL-B-68239), NLS7725 and variants thereof. In some embodiments, a methanotroph is selected from the group consisting of NLS1501, NLS1504 or NLS1508, and variants thereof. In some embodiments, a Methylobacterium is selected from the group consisting of LGP2020 (NRRL-B-67892), LGP2016 (NRRL B-67341), LGP2017 (NRRL B-67741), LGP2019 (NRRL B-67743), NLS7725, LGP2003 (NRRL B-50932), LGP2002 (NRRL B-50931), LGP2015 (NRRL B-67340), and variants thereof. In some embodiments, a methanotroph strain is NLS1501, NLS1504 or NLS1508 and a methylotroph strain is LGP2020 (NRRL-B-67892) or NLS7725. In some embodiment, a methytroph is a species selected from the group consisting of M. radiotolerans, M. populi and M. extorquens. In some embodiments, a methylotroph strain in the methods and compositions provided herein is selected from the group consisting of LGP2002 (NRRL B-50931), LGP2003 (NRRL B-50932), LGP2004 (NRRL B-50933), LGP2009 (NRRL B-50938), LGP2015 (NRRL B-67340), LGP2016 (NRRL B-67341), LGP2017 (NRRL B-67741), LGP2019 (NRRL B-67743), NLS0693 (NRRL B-67926), LGP2020 (NRRL-B-67892), and NLS7725. In some embodiments, a methanotroph is NLS1501 and a methylotroph is NLS7725. In some embodiments, a methanotroph is NLS1508 and a methylotroph is LGP2019 (NRRL B-67743).

In some embodiments, a method for mitigating methane comprises reducing methane concentration in a methane-containing media (e.g., manure or livestock waste) or fluid (e.g., any methane-containing gas or liquid such as methane-contaminated groundwater), the method comprising applying a composition comprising at least one methanotroph to the media or fluid. In some embodiments, the applied composition further comprises a methylotroph. In some embodiments the methylotroph is a Methylobacterium isolate capable of oxidizing methane and/or facilitating growth and function of the applied methanotoph strain or strains. In some embodiments, the methanotroph is selected from the group consisting of NLS1501, NLS1504, NLS1505, NLS1506, NLS1508, NLS1509, NLS1511, and NLS1512, and variants thereof. In some embodiments, a Methylobacterium in such embodiments is selected from the Methylobacterium strains provided in Table 1. In some embodiments, the Methylobacterium is selected from the group consisting of NLS0737, NLS0770, NLS5278, NLS5334, NLS5480, NLS5549, and variants thereof. In some embodiments, a methanotroph strain is NLS1501, NLS1504 or NLS1508 and a methylotroph strain is LGP2020 (NRRL-B-67892) or NLS7725. In some embodiment, a methytroph is a species selected from the group consisting of M. radiotolerans, M. populi and M. extorquens. In some embodiments, a methylotroph strain in the methods and compositions provided herein is selected from the group consisting of LGP2002 (NRRL B-50931), LGP2003 (NRRL B-50932), LGP2004 (NRRL B-50933), LGP2009 (NRRL B-50938), LGP2015 (NRRL B-67340), LGP2016 (NRRL B-67341), LGP2017 (NRRL B-67741), LGP2019 (NRRL B-67743), NLS0693 (NRRL B-67926), LGP2020 (NRRL-B-67892), and NLS7725. In some embodiments, a methanotroph is NLS1501 and a methylotroph is NLS7725. In some embodiments, a methanotroph is NLS1508 and a methylotroph is LGP2019 (NRRL B-67743).

In some embodiments, a method for mitigating methane comprises reducing methane emissions (e.g., in a landfill), the method comprising applying a first coating of a composition comprising at least one methanotroph, and optionally a Methylobacterium, to a first layer of material (e.g., overburden/soil or waste); at least partially covering the first layer and first coating with a second layer of material (e.g., overburden/soil or additional waste); applying a second coating of the composition comprising the at least one methanotroph, and optionally a Methylobacterium, to a second layer; and growing the methanotroph, and the Methylobacterium if present in the composition. In some embodiments, the methanotroph is selected from the group consisting of NLS1501, NLS1504, NLS1505, NLS1506, NLS1508, NLS1509, NLS1511, and NLS1512, and variants thereof. In some embodiments, where a Methylobacterium is also employed, the Methylobacterium is selected from the Methylobacterium strains provided in Table 1 herein, and variants thereof. In some embodiments, a methanotroph strain is NLS1501, NLS1504 or NLS1508 and a methylotroph strain is LGP2020 (NRRL-B-67892) or NLS7725. In some embodiment, a methytroph is a species selected from the group consisting ofM. radiotolerans, M. populi and M. extorquens. In some embodiments, a methylotroph strain in the methods and compositions provided herein is selected from the group consisting of LGP2002 (NRRL B-50931), LGP2003 (NRRL B-50932), LGP2004 (NRRL B-50933), LGP2009 (NRRL B-50938), LGP2015 (NRRL B-67340), LGP2016 (NRRL B-67341), LGP2017 (NRRL B-67741), LGP2019 (NRRL B-67743), NLS0693 (NRRL B-67926), LGP2020 (NRRL-B-67892), and NLS7725. In some embodiments, a methanotroph is NLS1501 and a methylotroph is NLS7725. In some embodiments, a methanotroph is NLS1508 and a methylotroph is LGP2019 (NRRL B-67743).

Also disclosed is a method for selecting a methanotroph isolate capable of utilizing methane as a food source, wherein the method comprises (a) selecting a methotroph isolate; (b) isolating the methotroph isolate; (c) detecting in the genome of the methanotroph isolate, a genetic element, wherein the genetic element comprises a component of a particulate methane monooxygenase; (d) treating a field, water, plant, plant part or seed with the methanotroph isolate, and (e) measuring green-house gas emissions.

Also disclosed are methods to identify combinations of methanotroph and methyltroph bacterial strains useful for mitigating methane gas, wherein said method comprises (a) selecting a methyltroph isolate; (b) selecting a methanotroph isolate; (c) isolating the methotroph isolate; (d) isolating a methylotroph isolate; (e) treating a field, water, plant, plant part or seed with said methanotroph and methylotroph combination isolates, (f) measuring green-house gas emissions; and (g) comparing said methanotroph and methylotroph combinations to a control treatment; (h) selecting identifying methanotroph and methylotroph combination that decreased methane levels in said emissions as compared to methane levels resulting from said control treatment. In some embodiments, a control treatment comprises application of a methanotroph in the absence of said methylotroph. In some embodiments, the methods further comprise the step of measuring the biomass and yield of said plant or said plant grown in a field treated with said methanotroph and methylotroph combination. In some embodiments plants treated with said methanotroph and methylotroph combination are grown in a low methane environment. In some embodiments, plants treated with said methanotroph and methylotroph combination are grown in a high methane environment. In some embodiments, a plant growth environment is a simulated rice paddy.

Also provided herein are recombinant constructs for expression of an pMMO component protein, or modification thereof, wherein said construct comprises a genetic element encoding any one or more of SEQ ID NO: 76-96 or a modification thereof. In some embodiments, recombinant constructs for expression of a pMMO component protein comprises an encoding sequence of any one or more of SEQ ID NOS:97-117.

### DETAILED DESCRIPTION

### Definitions

The term “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the terms “include,” “includes,” and “including” are to be construed as at least having the features or encompassing the items to which they refer while not excluding any additional unspecified features or unspecified items.

As used herein, the term “biological” refers to a component of a composition for treatment of plants or plant parts comprised of or derived from a microorganism. Biologicals include biocontrol agents, other beneficial microorganisms, microbial extracts, natural products, plant growth activators or plant defense agents. Non-limiting examples of biocontrol agents include bacteria, fungi, beneficial nematodes, and viruses.

As used herein “mitigate methane” refers to decreasing methane levels by reducing methane emissions or by enhancing removal of methane from sources of the gas, such as agricultural soil, wetlands, landfills, waste facilities, animal feed, water or air. Mitigation of methane may be the result of methane oxidation by the activity of pMMO and/or sMMO enzymes in the methanotrophic bacterial strains provided herein, or may be the result of secondary effects of the provided methanotroph and/or Methylobacterium strains on the microbiome of a treated plant or plant part.

As used herein, the term “methylotrophic bacteria” or “methylotroph” refers to genera and species of bacteria that are capable of using one-carbon compounds, other than methane, as their carbon source for growth. Methylotrophic bacteria include species in the genera Methylobacterium, Methylorubrum, Hyphomicrobium, Methylophilus, Methylobacillus, Methylophaga, Aminobacter, Methylorhabdus, Methylopila, Methylosulfonomonas, Marinosulfonomonas, Paracoccus, Xanthobacter, Ancylobacter (also known as Microcyclus), Thiobacillus, Rhodopseudomonas, Rhodobacter, Acetobacter, Bacillus, Mycobacterium, Arthrobacter, and Nocardia (Lidstrom, 2006). Methylotrophs as used herein refers to both obligate and facultative methylotrophs. Obligate methylotrophs refers to genera and species of bacteria that can only use one-carbon compounds, other than methane, as a carbon source for growth. Facultative methylotrophs refers to genera and species of bacteria that can use multi-carbon compounds, in addition to one-carbon compounds, as a carbon source for growth.

As used herein, the term “methanotrophic bacteria” or “methanotroph” refers to genera and species of bacteria that are capable of using methane as their carbon source for growth. Methanotrophic bacteria include species in the genera Methyloacidimicrobium, Methyloacidiplilum, Methylobacter, Methylocaldum, Methylocapsa, Methylocella, Methylococcus, Methylocystis, Methyloferula, Methylogaea, Methyloglobus, Methylohalobius, Methylomagnum, Methylomarinum, Methylomicrobium, Methylomonas, Methyloparacoccus, Methyloperedens, Methyloprofundus, Methylosarcina, Methylosinus, Methylosoma, Methylosphaera, Methylothermus, and Methylovulum.

As used herein, the term “Methylobacterium” refers to methylotroph genera and species in the methylobacteriaceae family, including bacterial species in the Methylobacterium genus and proposed Methylorubrum genus (Green and Ardley (2018)). Methylobacterium includes pink-pigmented facultative methylotrophic bacteria (PPFM) and also encompasses the non-pink-pigmented Methylobacterium nodulans, as well as colorless mutants of Methylobacterium isolates. For example, and not by way of limitation, “Methylobacterium” refers to bacteria of the species listed below as well as any new Methylobacterium species that have not yet been reported or described that can be characterized as Methylobacterium or Methylorubrum based on phylogenetic analysis: Methylobacterium adhaesivum; Methylobacterium oryzae; Methylobacterium aerolatum; Methylobacterium oxalidis; Methylobacterium aquaticum; Methylobacterium persicinum; Methylobacterium brachiatum; Methylobacterium phyllosphaerae; Methylobacterium brachythecii; Methylobacterium phyllostachyos; Methylobacterium bullatum; Methylobacterium platani; Methylobacterium cerastii; Methylobacterium pseudosasicola; Methylobacterium currus; Methylobacterium radiotolerans; Methylobacterium dankookense; Methylobacterium soli; Methylobacterium frigidaeris; Methylobacterium specialis; Methylobacterium fujisawaense; Methylobacterium tardum; Methylobacterium gnaphalii; Methylobacterium tarhaniae; Methylobacterium goesingense; Methylobacterium thuringiense; Methylobacterium gossipiicola; Methylobacterium trifolii; Methylobacterium gregans; Methylobacterium variabile; Methylobacterium haplocladii; Methylobacterium aminovorans (Methylorubrum aminovorans); Methylobacterium hispanicum; Methylobacterium extorquens (Methylorubrum extorquens); Methylobacterium indicum; Methylobacterium podarium (Methylorubrum podarium); Methylobacterium iners; Methylobacterium populi (Methylorubrum populi); Methylobacterium isbiliense; Methylobacterium pseudosasae (Methylorubrum pseudosasae); Methylobacterium jeotgali; Methylobacterium rhodesianum (Methylorubrum rhodesianum); Methylobacterium komagatae; Methylobacterium rhodinum (Methylorubrum rhodinum); Methylobacterium longum; Methylobacterium salsuginis (Methylorubrum salsuginis); Methylobacterium marchantiae; Methylobacterium suomiense (Methylorubrum suomiense; Methylobacterium mesophilicum; Methylobacterium thiocyanatum (Methylorubrum thiocyanatum); Methylobacterium nodulans; Methylobacterium zatmanii (Methylorubrum zatmanii); Methylobacterium symbiota; or Methylobacterium organophilum.

“Colonization efficiency” as used herein refers to the relative ability of a given microbial strain to colonize a plant host cell or tissue as compared to non-colonizing control samples or other microbial strains. Colonization efficiency can be assessed, for example and without limitation, by determining colonization density, reported for example as colony forming units (CFU) per mg of plant tissue, or by quantification of nucleic acids specific for a strain in a colonization screen, for example using qPCR.

As used herein “mineral nutrients” (also sometime referred to simply as “nutrients”) are micronutrients or macronutrients required or useful for plants or plant parts including for example, but not limited to, nitrogen (N), potassium (K), calcium (Ca), magnesium (Mg), phosphorus (P), and sulfur (S), and the micronutrients chlorine (Cl), Iron (Fe), Boron (B), manganese (Mn), zinc (Z), cobalt (Co), copper (Cu), molybdenum (Mo) and nickel (Ni).

As used herein, “vitamins” are organic compounds required in small amounts for normal growth and metabolism. Vitamins are important for human and/or animal growth and some vitamins have been reported to be beneficial to plants. Vitamins include but are not limited to vitamin A (including but not limited to all-trans-retinol, all-trans-retinyl-esters, as well as all-trans-beta-carotene and other provitamin A carotenoids), vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folic acid or folate), vitamin B12 (cobalamins), vitamin C (ascorbic acid), vitamin D (calciferols), vitamin E (tocopherols and tocotrienols), and vitamin K (quinones).

As used herein “fertilizer” can be a single nutrient nitrogen fertilizer, such as urea, ammonia or ammonia solutions (including ammonium nitrate, ammonium sulfate, calcium ammonium nitrate, and urea ammonium nitrate). In certain embodiments, the fertilizer can be a single nutrient phosphate fertilizer, such as a superphosphate or triple superphosphate or mixtures thereof, including double superphosphate. In certain embodiments, the fertilizer can be a single nutrient potassium-based fertilizer, such as muriate of potash. In certain embodiments, the compositions comprise multinutrient fertilizers including binary fertilizers (NP, NK, PK), including, for example monoammonium phosphate, diammonium phosphate, potassium nitrate and potassium chloride. In further embodiments, three-component fertilizers (NPK) providing nitrogen, phosphorus, and potassium are present in the aqueous compositions. In still further embodiments, the fertilizer comprises micronutrients, which may be chelated or non-chelated. In some embodiments, combinations of various fertilizers can be present in the aqueous solution, including combinations of nitrogen, phosphorus and/or micronutrient fertilizers. Nutrient solutions provided in hydroponic plant growth systems are also considered “fertilizers” in methods and compositions described herein.

As used herein, the term “strain” shall include all isolates of such strain.

As used herein, “variant” when used in the context of a methanotrophic bacterial isolate, refers to any isolate that has chromosomal genomic DNA with at least 99%, 99.9, 99.8, 99.7, 99.6%, or 99.5% sequence identity to chromosomal genomic DNA of a reference methanotrophic bacterial isolate, such as, for example, a deposited methanotrophic bacterial isolate provided herein. A variant of an isolate can be obtained from various sources including soil, plants or plant material, and water, particularly water associated with plants and/or agriculture. Variants include derivatives obtained from deposited isolates. Methanotrophic bacterial isolates or strains can be sequenced (for example as taught by Sanger et al. (1977), Bentley et al. (2008) or Caporaso et al. (2012)) and genome-scale comparison of the sequences conducted (Konstantinidis et al. (2005)) using sequence analysis tools, such as BLAST, as taught by Altschul et al. (1990) or clustalw (www.ebi.ac.uk/Tools/msa/clustalw2/).

As used herein, “derivative” when used in the context of a methanotrophic bacterial isolate, refers to any methanotrophic bacterial that is obtained from a deposited methanotrophic bacterial isolate provided herein. Derivatives of a methanotrophic bacterial isolate include, but are not limited to, derivatives obtained by selection, derivatives selected by mutagenesis and selection, and genetically transformed methanotrophic bacteria obtained from a methanotrophic bacterialisolate. A “derivative” can be identified, for example based on genetic identity to the strain or isolate from which it was obtained and will generally exhibit chromosomal genomic DNA with at least 99%, 99.9, 99.8, 99.7, 99.6%, or 99.5% sequence identity to chromosomal genomic DNA of the strain or isolate from which it was derived.

As used herein, “sequence identity” when used to evaluate whether a particular methanotrophic bacterial strain is a variant or derivative of a methanotrophic bacterial strain provided herein refers to a measure of nucleotide-level genomic similarity between the coding regions of two genomes. Sequence identity between the coding regions of bacterial genomes can be calculated, for example, by determining the Average Nucleotide Identity (ANI) score using FastANI (Jain et al. “High throughput ANI analysis of 90K prokaryotic genomes reveals clear species boundaries”, Nat Communications 9, 5114 (2018)) and Han et al. (“ANI tools web: a web tool for fast genome comparison within multiple bacterial strains”; Database, 2016, 1-5).

As used herein, a “correlation” is a statistical measure that indicates the extent to which two or more variables, here plant growth enhancement and identified genetic elements, occur together. A positive correlation indicates that a microbial strain containing a given genetic element is likely to enhance plant growth.

As used herein, a “pan-genome” is the entire set of genes for the microbial population being screened in a plant colonization efficiency screen. Thus, a pan-genome may represent the entire set of genes for a particular species, or the entire set of genes in multiple different species of the same genus or even the entire set of genes for multiple species classified in more than a single genus, where the strains in the population are from closely related genera.

As used herein a “genetic element” refers to an element in a DNA or RNA molecule that comprises a series of adjacent nucleotides at least 20 nucleotides in length and up to 50, 100, 1,000, or 10,000 or more, nucleic acids in length. A genetic element may comprise different groups of adjacent nucleic acids, for example, where the genome of a plant-associated microorganism contains introns and exons. The genetic element may be present on a chromosome or on an extrachromosomal element, such as a plasmid. In eukaryotic plant-associated microorganisms, the genetic element may be present in the nucleus or in the mitochondria. In some embodiments, the genetic element is a functional genetic element (e.g., a gene) that encodes a protein. Methods to detect genetic elements include, but are not limited to, techniques based on nucleic acid sequencing, nucleic acid hybridization, polymerase chain reactions, mass spectroscopy, nanopore based detection, branched DNA analyses, and combinations thereof.

As used herein, the terms “homologous” or “homologue” or “ortholog” refer to related genetic elements or proteins encoded by the genetic elements that are determined based on the degree of sequence identity. These terms describe the relationship between a genetic element or encoded protein found in one isolate, species or strain and the corresponding or equivalent genetic element or protein in another isolate, species or strain. As used herein, a particular genetic element in a first isolate, species or strain is considered equivalent to a genetic element present in a second isolate, species or strain when the proteins encoded by the genetic element in the isolates, species or strains have at least 50 percent identity. Percent identity can be determined using a number of software programs available in the art including BLASTP, ClustalW, ALLALIGN, DNASTAR, SIM, SEQALN, NEEDLE, SSEARCH and the like.

As used herein, the term “cultivate” means to grow a plant. A cultivated plant can be one grown and raised on a large agricultural scale or on a smaller scale, including for example a single plant.

As used herein, the term “hydroponic”, “hydroponics” or “hydroponically” refers to a method of cultivating plants in the absence of soil.

As used herein, the term “mitigating”, “mitigate”, or “mitigation” refers to a reduction of something or a combination of things.

As used herein, the term “methanotroph or methanotrophic bacteria” refers to genera and species that contains a pMMO gene.

Where a term is provided in the singular, other embodiments described by the plural of that term are also provided.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

### Further Description

Isolated methanotrophic bacteria (methanotrophs) are provided herein that oxidize methane, and can be formulated into compositions that can be used to mitigate methane in environments where methane is emitted or produced, such as in landfills, agricultural lands, wastewater treatment, wetlands, landfills, waste facilities, and dairy farms. Methanotrophic bacteria may also provide for additional benefits, for example in agricultural applications, where they can enhance early growth of plants, improve propagation/transplant vigor, increase nutrient uptake, improve stand establishment, improve stress tolerance, increase yield, and/or increase a plant's ability to utilize nutrients. In some embodiments, methanotrophs provide for nitrogen fixation, and or enhance nitrogen use efficiency of a treated plant. In some embodiments, application of methanotrophs results in increased yield at harvest, for example increased harvested seed yield. Compositions useful for treatment of fields, wasteland, animal feed, wetlands, landfills, waste, plants, seeds, or plant parts with such strains are provided herein. In some embodiments, methanotrophs are applied to rice plants resulting in decreased levels of methane and enhanced plant growth. In some embodiments, methanotrophs are applied to other crop plants, including, agricultural crop plants, for example row crops, such as corn, soybean, wheat, barley and millet, fruits and vegetables, leafy green plants, herbs, ornamentals, turf grasses, golf grass, shrubs, and trees. Strains of methanotrophic bacteria useful in the compositions and methods described herein include bacteria from a genus selected from the group consisting of Methyloacidimicrobium, Methyloacidiplilum, Methylobacter, Methylocaldum, Methylocapsa, Methylocella, Methylococcus, Methylocystis, Methyloferula, Methylogaea, Methyloglobus, Methylohalobius, Methylomagnum, Methylomarinum, Methylomicrobium, Methylomonas, Methyloparacoccus, Methyloperedens, Methyloprofundus, Methylosarcina, Methylosinus, Methylosoma, Methylosphaera, Methylothermus, and Methylovulum. In some embodiments, a methanotroph provided herein is a Methylocystis species selected from M. hirsuta, M. rosea and M. parvus. In some embodiments, a methanotroph provided herein is a Methylomicrobium lacus or Methylosarcina fibrate strain.

In certain embodiments, a methanotrophic bacteria is a Type II (Alphaproteobacteria) strain that comprises a pMMO2 methane monooxygenase encoded by an operon comprising expression sequences for pMMO2 protein components PmoA2, PmoB2 and PmoC2. In some embodiments, the Type II methanotroph is a Methylocystis species. In some embodiments, a methanotroph provided herein is a Methylocystis species selected from M. hirsuta, M. rosea and M. parvus. In some embodiments a methanotroph provided herein is a Methylocystis hirsuta isolate comprising PmoA2, PmoB2 and PmoC2 protein sequences of SEQ ID NOS: 76-78 or SEQ ID NOS: 79-81. In some embodiments, a Methylocystis hirsuta strain comprises a pMMO2 monooxygenase having PmoA2, PmoB2 and PmoC2 proteins with sequences at least 97, 98 or 99% identical to SEQ ID NOS:76-78 or SEQ ID NOS: 79-81. In some embodiments a Methylocystis hirsuta isolate is selected from the group consisting of NLS1505, NLS1506, NLS1508, NLS1509, NLS1511, NLS1512, variants thereof, or combinations thereof. Also provided are isolated methanotroph strains NLS1501, NLS1504, NLS1505, NLS1506, NLS1508, NLS1509, NLS1511, and NLS1512. In certain embodiments the methanotrophic bacteria in the composition has the ability to mitigate methane directly by oxidation of methane by pMMO. In some embodiments, Methylocystis hirsuta bacterial strains provided herein comprise sMMO proteins in addition to pMMO proteins. In some embodiments, methanotrophic bacterial strains facilitate oxidation of CH₄ into methanol (CH₃OH) followed by the incorporation of that carbon into bacterial biomass, or its oxidation to CO₂ and H₂O.

In some embodiments, methanotroph strains in the methods and compositions provided herein are Type I (Gammaproteobacter) strains. In some embodiments, Type I methanotrophs are species of Methylomicrobium or Methylosarcina. In some embodiments, a Methylomicrobium isolate comprises a PmoA protein at least 97, 98 or 99% identical to SEQ ID NO:83 or SEQ ID NO:84. In some embodiments, a Methylomicrobium isolate comprises a PmoB protein at least 97, 98 or 99% identical to SEQ ID NO:85 or SEQ ID NO:86. In some embodiments, a Methylomicrobium isolate comprises a PmoC protein at least 97, 98 or 99% identical to SEQ ID NO:87 or SEQ ID NO:88. In some embodiments, a Methylosarcina isolate comprises a PmoA protein at least 97, 98 or 99% identical to SEQ ID NO:89, SEQ ID NO:90 or SEQ ID NO:91. In some embodiments, a Methylosarcina isolate comprises a PmoB protein at least 97, 98 or 99% identical to SEQ ID NO:92 or SEQ ID NO:93. In some embodiments, a Methylosarcina isolate comprises a PmoC protein at least 97, 98 or 99% identical to SEQ ID NO:94, SEQ ID NO:95 or SEQ ID NO:96. In some embodiments the methanotrophs are isolates of Methylomicrobium lacus or Methylosarcina fibrata. In some embodiments, a Methylomicrobium lacus isolate is NLS1501. In some embodiments a Methylosarcina fibrata isolate is NLS1504. In some embodiments, methanotroph bacterial strains provided herein comprise sMMO proteins in addition to pMMO proteins. In some embodiments, a methanotroph strain for use in the compositions and methods provided herein comprises a 16S encoding sequence of any one of SEQ ID NO:118-120.

In certain embodiments, the methanotroph in the composition is NLS1501, NLS1504, NLS1505, NLS1506, NLS1508, NLS1509, NLS1511, NLS1512, variants thereof, or a combination thereof. In certain embodiments, the methanotroph in the composition has the ability to use methane as a carbon source for growth. Such strains find use as described herein for mitigating methane production, for example in agricultural applications, including plant production in flooded fields, for reducing methane produced in animal production, such as cattle or dairy industries, or for reducing natural methane sources such as exist in wetlands or other natural water sources, (including but not limited to lakes, rivers, mangroves, marshes, bogs and streams), in geological sources, or in gases produced as the result of wildfires, wild animals, or insects. Such strains find use as described herein for mitigating methane production, for example in wetland, landfill, or waste applications for reducing methane produced in animal production, such as cattle or dairy industries, or for reducing natural methane sources such as exist in wetlands or other natural water sources, (including but not limited to lakes, rivers, marshes, bogs and streams), in geological sources, or in gases produced as the result of wildfires, wild animals, or insects. By reducing methane resulting from such practices or present in such sources, the concentration of atmospheric greenhouse gases can be reduced and decrease the potential for methane to have detrimental effects, particularly in contributing to global warming. In some embodiments, methanotroph strains provided herein not only mitigate methane levels associated with agricultural crop production, but also provide additional benefits to a treated plant.

Further provided are methods of improving production of plants by treatment with one or more Type II or Type I methanotroph strains provided herein. In certain embodiments, treated plants are grown in a field, an irrigated or flooded field, hydroponically or in an aeroponic plant cultivation system. Such plants can be without limitation, agricultural crop plants, including without limitation corn, soybean, rice, millet, canola, and wheat, fruits and vegetables, leafy green plants, herbs, ornamentals, turf grasses, golf grass, shrubs, and trees. In certain embodiments, the methanotroph in the composition is NLS1501, NLS1504, NLS1505, NLS1506, NLS1508, NLS1509, NLS1511, NLS1512, variants thereof, or a combination thereof.

In some embodiments, a treated plant is a corn or rice plant and the methanotroph is selected from the group consisting of NLS1501, NLS1504 and NLS1508. In some embodiments, production is improved by enhanced early growth of treated plants or plants grown from treated seeds in comparison to an untreated control plant or in comparison to a control plant grown from an untreated seed. Such enhanced early growth is measured, for example, by an increase in biomass of treated plants, including increased shoot, leaf, root, or whole seedling biomass. Increased early growth can result in various improvements in plant production, including for example increased biomass production or yield of harvested plants, increased and/or more uniform fruit production, faster seed set, earlier maturation, increased rate of leaf growth, increased rate of root growth, increased seed yield, and decreased cycle time in comparison to an untreated control plant or in comparison to a control plant grown from an untreated seed. In certain embodiments, application of methanotroph strains as provided herein provides for a 1%, 2%, 3%, 4%, 5%, 6%, 7%, 8%, 9%, 10%, 12%, 15%, 17%, 20%, 30% or 40% increase in any of the aforementioned traits in comparison to an untreated control plant or in comparison to a control plant grown from an untreated seed. In some embodiments, production is enhanced by increased rooting, for example of plant cuttings, where such increased rooting can result in decreased cycling time and/or increased biomass or yield of the treated plants.

In some embodiments of methods provided herein, a pasture, wasteland or field is treated. In some embodiments of methods provided herein, treatment is done in a waste facility. In some embodiments of method provided herein, the field is flooded or irrigated. In some embodiments of the method provided herein, a plant seed is treated. In certain other embodiments, a plant seedling or part thereof is treated. In some embodiments, a plant shoot or seedling is treated.

Various methods for identifying a methanotroph strain that mitigates methane are also provided herein. In one method, a wetland, field, plant, plant part or seed is treated with at least a first methanotroph strain and methane emissions measured and compared to emissions from control strains and/or other tested strains to identify strains that mitigate methane. In some embodiments, a control strain is a Methylocystis strain that does not contain pMMO2, such as NLS1500. In some embodiments, a methanotroph strain useful for methane mitigation comprises genetic elements encoding one or more of the PmoA, PmoB and PmoC proteins provided herein as SEQ ID NOS:76-96. In some embodiments, a genetic element encoding a PmoA, PmoB and PmoC protein has a nucleotide sequence of SEQ ID NOS:97-117. In some embodiments, a methanotroph strain useful for methane mitigation comprises a 16S sequence of SEQ ID NO:118-120.

In some embodiments of compositions and methods provided herein, a combination of a methanotroph strain and one or more methylotroph bacterial strains are employed to improve plant production and/or mitigate methane. In some embodiments, useful methylotrophic bacterial strains are from a species selected from the group consisting of Methylobacterium, Methylorubrum, Hyphomicrobium, Methylophilus, Methylobacillus, Methylophaga, Aminobacter, Methylorhabdus, Methylopila, Methylosulfonomonas, Marinosulfonomonas, Paracoccus, Xanthobacter, Ancylobacter (also known as Microcyclus), Thiobacillus, Rhodopseudomonas, Rhodobacter, Acetobacter, Bacillus, Mycobacterium, Arthobacter, and Nocardia. In some embodiments, methylotrophic bacteria in the compositions and methods provided herein are species of Methylobacterium or Methylorubrum. As shown herein, application to plants of compositions comprising methanotrophs and methyltrophs, such as Methylobacterium or Methylorubrum, results in methane mitigation and enhanced growth and yield of treated plants. Without being limited by way of explanation, methylotrophic bacteria may enhance growth and yield of treated plants directly, but may also enhance growth and activity of applied methanotroph strains by consuming compounds (for example, waste products) produced by methanotrophs, thus allowing the methanotrophs to grow and function more efficiently. In some embodiments, a methylotroph that enhances activity of a methanotrophic bacteria is a Methylobacterium strain provided in Table 1. In some embodiments the Methylobacterium strain is a M. radiotolerans, M. extorquens or a M. populi strain. In some embodiments, the Methylobacterium strain is selected from the group consisting of LGP2019 (NRRL B-67743), LGP2020 (NRRL-B-67892), and NLS7725

In some embodiments, a composition comprising one or more methanotroph and methane oxidizing Methylobacterium strains that mitigate or decrease methane from agriculture lands, also impart a trait improvement to said plant selected from increased biomass production, decreased cycle time, increased rate of leaf growth, decreased time to develop two true leaves, increased rate of root growth, increased nutrients, and increased seed yield. In some embodiments, additional trait improvements are provided by the presence of nif genes for nitrogen fixation in a methanotroph strain. In some embodiments, enhanced nitrogen use efficiency is provided by a Methylobacterium strain. In some embodiments a Methylobacterium strain that enhances nitrogen use efficiency is selected from the group consisting of LGP2001 (NRRL B-50930), LGP2002 (NRRL B-50931), LGP2009 (NRRL B-50938), LGP2015 (NRRL B-67340), LGP2016 (NRRL B-67341), LGP2017 (NRRL B-67741), LGP2018 (NRRL B-67742), LGP2019 (NRRL B-67743), NLS0693 (NRRL B-67926), LGP2167 (NRRL B-67927), LGP2020 (NRRL-B-67892), LGP2021 (NRRL-B-68032), LGP2022 (NRRL-B-68033), LGP2023 (NRRL-B-68034), LGP2029 (NRRL B-68065), LGP2030 (NRRL B-68066), LGP2031 (NRRL B-68067), LGP2033 (NRRL B-68068), LGP2034 (NRRL B-68069), NLS0665 (NRRL B-68194), NLS0754 (NRRL B-68197), NLS0672 (NRRL B-68196), NLS0729 (NRRL B-68195), NLS0049 (NRRL-B-68236), NLS0591 (NRRL-B-68215), NLS0439 (NRRL-B-68216), NLS1310 (NRRL-B-68217), NLS1312 (NRRL-B-68218), NLS0612 (NRRL-B-68237), NLS0706 (NRRL-B-68238), NLS0725 (NRRL-B-68239), NLS7725, and variants thereof.

In some embodiments, methanotrophic bacterial strains and/or methylotroph strains in compositions provided herein contribute to methane mitigation by impacting other microbial populations and/or the activity of other microbes in the plant environment, for example by enhancing growth and activity of methanotrophs present in an environment, or by decreasing activity or populations of methanogens present in the environment.

Also disclosed is a method for selecting a methanotroph isolate capable of utilizing methane as a food source, wherein the method comprises (a) selecting a methotroph isolate; (b) isolating the methotroph isolate; (c) detecting in the genome of the methanotroph isolate, a genetic element, wherein the genetic element comprises a component of a particulate methane monooxygenase; (d) treating a field, water, plant, plant part or seed with the methanotroph isolate, and (e) measuring green-house gas emissions. In some embodiments, a treated plant is grown in a low methane environment to identify reduction in methane gas. In further embodiments, additional plant production improvements, including enhanced plant growth and yield, are evaluated in such a method. In some embodiments, enhanced plant growth and yield resulting from treatment with one or more methanotrophs is evaluated for plants growing in a high methane environments. In some embodiments, a low methane environment is one in which methane gas is produced at less than 20 kg/acre. In some embodiments, a high methane environment is one in which methane gas is produced at greater than 100 kg/acre. In this manner, a methanotroph strain or strains is identified and selected, wherein the strain provides for reduction of methane produced during growth of a treated cultivated plant or a plant part in comparison to an untreated control plant or plant part. Such methods may also be used for identification of combinations of one or more methanotroph and methylotroph strains that reduce methane emissions and enhance plant growth and biomass.

In other embodiments, the ability of a methanotroph strain or a combination of one or more methanotroph and methylotroph strains to enhance nitrogen use efficiency and enhance growth of treated plants as a results of increased NUE are identified. In some embodiments, a rice seed is treated. In some embodiments, field, plants, seeds or seedlings are separately treated with two, three, four or more methanotroph strains and growth and nitrogen content are compared for plants or plant parts treated with different strains, and a methanotroph strain or strains demonstrating increased yield or nitrogen content and/or increased growth under nitrogen limited conditions is selected and identified as providing for enhanced nitrogen use efficiency. In other embodiments, methanotroph strains are applied to seeds for planting and plants grown under nitrogen limited conditions are harvested to determine effect of the strain on plant yield.

Various methods of using methanotrophic bacteria or a combination of a methanotroph strain and Methylobacterium strains to mitigate methane, enhance early growth or rooting, improve propagation/transplant vigor, increase nutrient uptake, improve stand establishment, improve stress tolerance and/or increase a plant's ability to uptake and/or utilize nutrients, such as nitrogen, potassium, sulfur, cobalt, copper, zinc, phosphorus, boron, iron and manganese in plants, such as leafy green plants, row crops, ornamentals, turf grasses, golf grasses, shrubs, Cannabis and other specialty crops are provided herein. In certain embodiments, methanotroph treatment of a row crop, including but not limited to corn, soybean, rice, millet, canola, and wheat, results in enhanced plant growth and yield. In some embodiments, a methanotroph strain is NLS1501, NLS1504 or NLS1508 and a methylotroph strain is LGP2019 (NRRL B-67743), LGP2020 (NRRL-B-67892) or NLS7725. In some embodiment, a methytroph is a species selected from the group consisting of M. radiotolerans, M. populi and M. extorquens. In some embodiments, a methylotroph strain in the methods and compositions provided herein is selected from the group consisting of LGP2002 (NRRL B-50931), LGP2003 (NRRL B-50932), LGP2004 (NRRL B-50933), LGP2009 (NRRL B-50938), LGP2015 (NRRL B-67340), LGP2016 (NRRL B-67341), LGP2017 (NRRL B-67741), LGP2019 (NRRL B-67743), NLS0693 (NRRL B-67926), LGP2020 (NRRL-B-67892), and NLS7725. In some embodiments, a methanotroph is NLS1508 and a methylotroph is LGP2019 (NRRL B-67743). In some embodiments, a methanotroph is NLS1501 and a methylotroph is NLS7725. In some embodiments, a methanotroph is NLS1501 and a methylotroph is LGP2020 (NRRL-B-67892). In certain embodiments, the treated crop is rice and the methanotroph is selected from the group consisting of NLS1501, NLS1504, NLS1505, NLS1506, NLS1508, NLS1509, NLS1511, NLS1512, variants thereof, or a combination thereof. In certain embodiments, a treatment includes a methanotroph and one or more Methylobacterium provided in Table 1. In certain embodiments, a treatment includes a methanotroph and one or more Methylobacterium, wherein the one or more Methylobacterium mitigates methane and/or enhances rice yield. A Methylobacterium strain that mitigates methane is selected from the group consisting of NLS0707, NLS0737, NLS5278, NLS5334, NLS5480, NLS5549, and variants thereof. A Methylobacterium strain that enhances rice yield is selected from the group consisting of LGP2016, LGP2017, LGP2019, and LGP2020. In certain embodiments, methanotroph treatment of soil, agriculture land, including a field or a flooded and irrigated field, a seed, a leaf, a stem, a root, or a shoot can enhance early growth, propagation/transplant vigor, stand establishment, and/or stress tolerance as well as or alternatively enhance nutrient use efficiency.

In some embodiments, a methanotroph is NLS1508 and a methylotroph is LGP2019 (NRRL B-67743), in some embodiments, a methanotroph is NLS1508 and a methylotroph is LGP2020 (NRRL-B-67892), in some embodiments, a methanotroph is NLS1508 and a methylotroph is NLS7725. In some embodiments, a methanotroph is NLS1501 and a methylotroph is LGP2019 (NRRL B-67743), in some embodiments, a methanotroph is NLS1501 and a methylotroph is LGP2020 (NRRL-B-67892), in some embodiments, a methanotroph is NLS1501 and a methylotroph is NLS7725. In some embodiments, a methanotroph is NLS1504 and a methylotroph is LGP2019 (NRRL B-67743), in some embodiments, a methanotroph is NLS1504 and a methylotroph is LGP2020 (NRRL-B-67892), in some embodiments, a methanotroph is NLS1504 and a methylotroph is NLS7725. In some embodiments, a methanotroph is NLS1501 and a methylotroph is NLS7725. In some embodiments, a methanotroph is NLS1501 and a methylotroph is LGP2020 (NRRL-B-67892). In some embodiments, a methanotroph is NLS1508 and a methylotroph is LGP2019 (NRRL B-67743). In some embodiments, a methanotroph is NLS1501 and a methylotroph is NLS7725. In some embodiments, a methanotroph is NLS1501 and a methylotroph is LGP2020 (NRRL-B-67892).

Alternatively, compositions comprising such methanotrophs, and optionally one or more methylotroph strains may be applied to soil or other growth medium where plants are grown. Methanotroph and optionally methylotroph soil treatments or applications can include, but are not limited to, fields (e.g. flooded or irrigated fields), in-furrow applications (e.g., before, during, and/or after seed deposition), soil drenches, distribution of granular or other dried formulations to the soil (e.g., before, during, and/or after seed deposition or plant growth). Treatments for plants grown in hydroponic systems can include seed treatments prior to germination, foliar applications to germinated plants or parts thereof, and applications in a liquid solution used in the hydroponic system. In certain embodiments, treatment of a plant can include application to the seed, plant, and/or a part of the plant and can thus comprise any methanotroph treatment or application resulting in colonization of the plant by the methanotroph. In some embodiments, application of one or more methanotrophs and optionally one or more methylotrophs to crops that are propagated by cutting can enhance growth and/or rooting of such plants. Field transplants of such treated and rooted cuttings may demonstrate decreased cycling time, and/or improved biomass and/or yield as a result of such treatments.

Treatments or applications to plants described herein can include, but are not limited to, spraying, coating, partially coating, immersing, drenching, and/or imbibing the field, seed, plant or plant parts with the methanotroph, and optionally one or more methylotroph, strains, or compositions comprising such strains. In certain embodiments, soil, a seed, a leaf, a stem, a root, a tuber, or a shoot can be sprayed, immersed drenched and/or imbibed with a liquid, semi-liquid, emulsion, or slurry of a composition provided herein. In some embodiments, one or more methanotroph strains may be applied together or separately with one or more methylotroph strains. In some embodiments, methanotroph, and optionally methylotroph strains, are applied to multiple plant parts and/or at multiple stages of plant growth. In certain embodiments, methane oxidizing methanotrophs described herein are applied as foliar sprays or seed treatments to row crops. In some embodiments, the crop is corn and a methanotroph is applied as a seed treatment. In some embodiments, the corn crop is grown under nitrogen limited conditions and the ability of the applied methanotroph to enhance NUE is observed. In some embodiments, corn seeds are treated in a plant box application with NLS1508. In some embodiments, the crop is rice, and plants are treated with an initial foliar application at a flooded stage. In some embodiments, foliar applications are made when a rice paddy is at full flood stage. In some embodiments, additional foliar applications of methanotroph are made. In some embodiments, a second foliar application of methanotroph is made from 20-40 days following the initial application. In some embodiments, methanotroph is also applied as a foliar spray prior to the booting stage of development (characterized by swelling of the flag leaf sheath caused by an increase in the size of the panicle). In some embodiments, a foliar spray is applied 14 days prior to booting stage. In some embodiments, a methanotroph is applied initially as a foliar spray at full flood stage, followed by a second foliar application approximately 4-6 weeks later, for example around 30 days later. In some embodiments, a third foliar application of a methanotroph is made not later than 14 days prior to booting stage. In some embodiments, a methanotroph applied as a foliar spray to rice is NLS1501. In some embodiments, a methanotroph and a methylotroph are applied to rice. In some embodiments, the methanotroph is applied as a foliar application and a methylotroph is applied as a seed treatment. In some embodiments, the methanotroph is NLS1501 and the methylotroph is LGP2019 (NRRL B-67743).

Such treatments, applications, seed immersion, or imbibition can be sufficient to provide for mitigation of green-house gas emissions, enhanced early growth and/or increased levels of one or more mineral nutrients and/or vitamins content in harvestable tissue from a treated plant or plant grown from a treated seed in comparison to an untreated plant or plant grown from an untreated seed. Enhanced early growth can lead to further improvements in plant production including an increase in biomass of treated plants, such as increased shoot, root, or whole seedling biomass. Enhanced early growth can result in various additional improvements in plant production, including for example increased yield of harvested plants or harvested plant parts, increased and/or more uniform fruit production, faster seed set, earlier maturation, increased rate of leaf growth, increased rate of root growth, increased seed yield, and decreased cycle time. In certain embodiments, plant seeds or cuttings can be immersed and/or imbibed for at least 1, 2, 3, 4, 5, or 6 hours. Such immersion and/or imbibition can, in certain embodiments, be conducted at temperatures that are not deleterious to the plant seed or the methanotroph. In certain embodiments, the seeds can be treated at about 15 to about 30 degrees Centigrade or at about 20 to about 25 degrees Centigrade. In certain embodiments, seed imbibition and/or immersion can be performed with gentle agitation. Seed treatments can be effected with both continuous and/or batch seed treaters. In certain embodiments, the coated seeds can be prepared by slurrying seeds with a coating composition comprising a methanotroph strain that increases the levels of one or more mineral nutrients and/or vitamins and air-drying the resulting product. Air-drying can be accomplished at any temperature that is not deleterious to the seed or the methanotroph, but will typically not be greater than 30 degrees Centigrade. The proportion of coating that comprises the methanotroph strain includes, but is not limited to, a range of 0.1 to 25% by weight of the seed or other plant part, 0.5 to 5% by weight of the seed or other plant part, and 0.5 to 2.5% by weight of the seed or other plant part. In certain embodiments, a solid substance used in the seed coating or treatment will have a methanotroph strain that increases mineral nutrient and or vitamin content adhered to a solid substance as a result of being grown in biphasic media comprising the methanotroph strain, solid substance, and liquid media.

In certain embodiments where plant seeds are treated with methanotroph compositions provided herein, the compositions further comprise one or more lubricants to ensure smooth flow and separation (singulation) of seeds in the seeding mechanism, for example a planter box. Lubricants for use in such compositions include talc, graphite, polyethylene wax based powders (such as Fluency Agent), protein powders, for example soybean protein powders, or a combination of protein powders and a lipid, for example lecithin or a vegetable oil. Lubricants can be applied to seeds simultaneously with application of a methanotroph, or may be mixed with a methanotroph prior to application of the compositions to the seeds.

In certain embodiments, treated plants are cultivated in a hydroponic system. In some embodiments, plant seeds are treated and plants are grown from the treated seeds continuously in the same cultivation system. In some embodiments, plant seeds are treated and cultivated in a hydroponic nursery to produce seedlings. The seedlings transferred to a different hydroponic system, for example for commercial production of leafy greens. In some embodiments, a methanotroph strain that enhances early growth or increases the levels of one or more mineral nutrients and/or vitamins persists in the seedlings transferred to a greenhouse production system and continues to provide advantages such as improved micronutrient and/or vitamin content and/or biomass production, through the further growth of the leafy green plant. In some embodiments, plant seedlings transferred to a greenhouse production system may be further treated with LGP2009, LGP2022, LGP2023, LGP2021, LGP2033 or variants thereof, or with one or more other Methylobacterium strains that increase the levels of one or more mineral nutrients and/or vitamins prior to, during or after transfer to the production system.

Some methanotrophs are present in soil samples that are collected from various sources, particularly rice fields. For example NLS1501 is known to be present at a detectable level in soil samples prior to addition of an isolated NLS1501 sample. Even though the sample contains a detectable level of a methanotroph, treating the soil or plants grown in the soil with a known titer of the methanotroph shows significant improvement in plant growth and/or reduction in methane release.

In certain embodiments, the composition used to treat the pasture, wasteland, field, seed, plant, or plant part can contain a methanotroph strain and an agriculturally acceptable excipient. Agriculturally acceptable excipients include, but are not limited to, woodflours, clays, activated carbon, diatomaceous earth, fine-grain inorganic solids, calcium carbonate and the like. Clays and inorganic solids that can be used with the include, but are not limited to, calcium bentonite, kaolin, china clay, talc, perlite, mica, vermiculite, silicas, quartz powder, montmorillonite and mixtures thereof. Agriculturally acceptable excipients also include various lubricants such as talc, graphite, polyethylene wax based powders (such as Fluency Agent), protein powders, for example soybean protein powders, or a combination of protein powders and a lipid, for example lecithin or a vegetable oil.

Preferably, the agriculturally acceptable adjuvant comprises kaolin, talc, graphite, mica, vermiculite, soyobean protein powder, or a combination thereof.

Agriculturally acceptable adjuvants that promote sticking to the seed that can be used include, but are not limited to, polyvinyl acetates, polyvinyl acetate copolymers, hydrolyzed polyvinyl acetates, polyvinylpyrrolidone-vinyl acetate copolymer, polyvinyl alcohols, polyvinyl alcohol copolymers, polyvinyl methyl ether, polyvinyl methyl ether-maleic anhydride copolymer, waxes, latex polymers, celluloses including ethylcelluloses and methylcelluloses, hydroxy methylcelluloses, hydroxypropylcellulose, hydroxymethylpropylcelluloses, polyvinyl pyrrolidones, alginates, dextrins, malto-dextrins, polysaccharides, fats, oils, proteins, karaya gum, jaguar gum, tragacanth gum, polysaccharide gums, mucilage, gum arabics, shellacs, vinylidene chloride polymers and copolymers, soybean-based protein polymers and copolymers, lignosulfonates, acrylic copolymers, starches, polyvinylacrylates, zeins, gelatin, carboxymethylcellulose, chitosan, polyethylene oxide, acrylamide polymers and copolymers, polyhydroxyethyl acrylate, methylacrylamide monomers, alginate, ethylcellulose, polychloroprene and syrups or mixtures thereof. Other useful agriculturally acceptable adjuvants that can promote coating include, but are not limited to, polymers and copolymers of vinyl acetate, polyvinylpyrrolidone-vinyl acetate copolymer and water-soluble waxes. Further, agriculturally acceptable adjuvants also include various lubricants (which can provide for smooth flow and separation (singulation) of seeds) such as talc, graphite, polyethylene wax based powders (such as Fluency Agent), protein powders, for example soybean protein powders, or a combination of protein powders and a lipid, for example lecithin or a vegetable oil. Various surfactants, dispersants, anticaking-agents, foam-control agents, and dyes disclosed herein and in U.S. Pat. No. 8,181,388 can be adapted for use with compositions comprising a suitable methanotroph strain. In certain embodiments, the seed and/or seedling is exposed to the composition by providing the methanotroph strain in soil in which the plant or a plant arising from the seed are grown, or other plant growth media in which the plant or a plant arising from the seed are grown. Examples of methods where the methanotroph strain is provided in the field and soil include in furrow applications, soil drenches, and the like.

Preferably, agriculturally acceptable adjuvants that promote sticking to the seed are celluloses dextrins, maltodextrins, polysaccharides, polysaccharide gums, or a combination thereof.

The agriculturally acceptable adjuvant can be present in the composition at a concentration of from 0 wt. % to about 95 wt. %, from about 0.1 wt. % to about 95 wt. %, from about 0.5 wt. % to about 95 wt. %, from about 1 wt. % to about 95 wt. %, from about 2 wt. % to about 95 wt. %, from about 3 wt. % to about 95 wt. %, from about 4 wt. % to about 95 wt. %, from about 5 wt. % to about 95 wt. %, from about 0.1 wt. % to about 90 wt. %, from about 0.5 wt. % to about 90 wt. %, from about 1 wt. % to about 90 wt. %, from about 2 wt. % to about 90 wt. %, from about 3 wt. % to about 90 wt. %, from about 4 wt. % to about 90 wt. %, from about 5 wt. % to about 90 wt. %, from about 0.1 wt. % to about 85 wt. %, from about 0.5 wt. % to about 85 wt. %, from about 1 wt. % to about 85 wt. %, from about 2 wt. % to about 85 wt. %, from about 3 wt. % to about 85 wt. %, from about 4 wt. % to about 85 wt. %, from about 5 wt. % to about 85 wt. %, from about 0.1 wt. % to about 80 wt. %, from about 0.5 wt. % to about 80 wt. %, from about 1 wt. % to about 80 wt. %, from about 2 wt. % to about 80 wt. %, from about 3 wt. % to about 80 wt. %, from about 4 wt. % to about 80 wt. %, or more preferably, from about 5 wt. % to about 80 wt. %,

In certain embodiments, compositions comprising methanotrophs and optionally Methylobacterium strains provided herein or variants thereof will also find use in treatment of plants to mitigate methane and/or enhance early plant growth and/or plant yield. Treated plants include, for example rice and other field crops, ornamentals, turf grasses, golf grasses, shrubs, and trees grown in commercial production, such as conifer trees. Without limitation, such additional plant species include corn, soybean, cruciferous or Brassica sp. (e.g., B. napus, B. rapa, B. juncea), including vegetable Brassica sp., alfalfa, rice, rye, wheat, barley, oats, sorghum, millet (e.g., pearl millet (Pennisetum glaucum), proso millet (Panicum miliaceum), foxtail millet (Setaria italica), and finger millet (Eleusine coracana)), sunflower, safflower, tobacco, potato, peanuts, cotton, species in the genus Cannabis (including, but not limited to, Cannabis sativa and industrial hemp varieties), alfalfa, clover, cover-crops, sweet potato (Ipomoea batatus), cassava, coffee, coconut, ornamentals (including, but not limited to, azalea, hydrangea, hibiscus, roses, tulips, daffodils, petunias, carnation, poinsettia, and chrysanthemum), conifers (including, but not limited to pines such as loblolly pine, slash pine, ponderosa pine, lodge pole pine, and Monterey pine; Douglas-fir; Western hemlock; Sitka spruce; redwood; true firs such as silver fir and balsam fir; cedars such as Western red cedar and Alaska yellow-cedar) and turfgrass (including, but are not limited to, annual bluegrass, annual ryegrass, Canada bluegrass, fescue, bentgrass, wheatgrass, Kentucky bluegrass, orchard grass, ryegrass, redtop, Bermuda grass, St. Augustine grass, and zoysia grass); fruit (including but not limited to citrus, pome, and tropical fruit); nuts; and tea. Leafy green plants that can be treated include vegetable crop with edible leaves, for example, spinach, kale, lettuce (including but not limited to romaine, butterhead, iceberg and loose leaf lettuces), collard greens, cabbage, beet greens, watercress, swiss chard, arugula, escarole, endive, bok choy and turnip greens. Leafy green plants as used herein also refers to plants grown for harvest of microgreens and/or herbs, including but not limited to lettuce, cauliflower, broccoli, cabbage, watercress, arugula, garlic, onion, leek, amaranth, swill chard, been, spinach, melon, cucumber, squash, basil, celery, cilantro, radish, radicchio, chicory, dill, rosemary, French tarragon, basil, Pennisetum, carrot, fennel, beans, peas, chickpeas, and lentils.

In certain embodiments, a methanotroph or Methylobacterium strain used to treat a given cultivar or variety of plant seed, plant or plant part can be a strain that was isolated from a different plant species, or a different cultivar or variety of the plant species being treated, and is thus heterologous or non-resident to the treated plant or plant part.

In certain embodiments, a manufactured combination composition comprising two or more methanotroph strains or a combination of one or more methanotroph strains with one or more Methylobacterium strains can be used to treat a field, seed or plant part in any of the methods provided herein. Such manufactured combination compositions can be made by methods that include harvesting monocultures of each strain and mixing the harvested monocultures to obtain the manufactured combination composition. In certain embodiments, the manufactured combination composition of one or more methanotrophs and optionally one or more Methylobacterium strains can comprise a methanotroph and Methylobacterium strains isolated from different plant species or from different cultivars or varieties of a given plant.

In certain embodiments, a manufactured combination composition comprising one or more methanotroph strains and a second biological can be used to treat a field, seed or plant part in any of the methods provided herein. Such manufactured combination compositions can be made by methods that include harvesting monocultures of each strain and mixing the harvested monocultures to obtain the manufactured combination composition of methanotrophs. In certain embodiments, the manufactured combination composition of a methanotroph and the second biological can comprise isolates from different plant species or from different cultivars or varieties of a given plant. In certain embodiments, a manufactured combination composition comprising one or more methanotroph strains and a Methylobacterium can be used to treat a field, seed or plant part in any of the methods provided herein.

In certain embodiments, an effective amount of the methanotroph or Methylobacterium strain or strains used in treatment of plants, seeds or plant parts is a composition having a titer of at least about 1×10⁶ colony-forming units per milliliter, at least about 5×10⁶ colony-forming units per milliliter, at least about 1×10⁷ colony-forming units per milliliter, at least about 5×10⁸ colony-forming units per milliliter, at least about 1×10⁹ colony-forming units per milliliter, at least about 1×10¹⁰ colony-forming units per milliliter, or at least about 3×10¹⁰ colony-forming units per milliliter. In certain embodiments, an effective amount of the strain or strains is a composition with the methanotroph at a titer of about least about 1×10⁶ colony-forming units per milliliter, at least about 5×10⁶ colony-forming units per milliliter, at least about 1×10⁷ colony-forming units per milliliter, or at least about 5×10⁸ colony-forming units per milliliter to at least about 6×10¹⁰ colony-forming units per milliliter of a liquid or an emulsion. In certain embodiments, an effective amount of the methanotroph strain or strains is a composition with the methanotroph at least about 1×10⁶ colony-forming units per gram, at least about 5×10⁶ colony-forming units per gram, at least about 1×10⁷ colony-forming units per gram, or at least about 5×10⁸ colony-forming units per gram to at least about 6×10¹⁰ colony-forming units of methanotroph per gram of the composition. In certain embodiments, an effective amount of a composition provided herein can be a composition with a methanotroph titer of at least about 1×10⁶ colony-forming units per gram, at least about 5×10⁶ colony-forming units per gram, at least about 1×10⁷ colony-forming units per gram, or at least about 5×10⁸ colony-forming units per gram to at least about 6×10¹⁰ colony-forming units of methanotroph per gram of particles in the composition containing the particles that comprise a solid substance wherein a mono-culture or co-culture of methanotroph strain or strains is adhered thereto. In certain embodiments, an effective amount of a composition provided herein to a plant or plant part can be a composition with a methanotroph titer of at least about 1×10⁶ colony-forming units per mL, at least about 5×10⁶ colony-forming units per mL, at least about 1×10⁷ colony-forming units per mL, or at least about 5×10⁸ colony-forming units per mL to at least about 6×10¹⁰ colony-forming units of methanotroph per mL in a composition comprising an emulsion wherein a mono-culture or co-culture of a methanotroph strain or strains adhered to a solid substance is provided therein or grown therein. In certain embodiments, an effective amount of a composition provided herein can be a composition with a methanotroph titer of at least about 1×10⁶ colony-forming units per mL, at least about 5×10⁶ colony-forming units per mL, at least about 1×10⁷ colony-forming units per mL, or at least about 5×10⁸ colony-forming units per mL to at least about 6×10¹⁰ colony-forming units of methanotroph per mL in a composition comprising an emulsion wherein a mono-culture or co-culture of a methanotroph strain or strains is provided therein or grown therein. Where a second biological, such as a Methylobacterium strain is present in the composition, the second biological will be present at similar titers as noted above for methanotrophs.

In certain embodiments, an effective amount of a methanotroph strain or strains that provides for mitigation of green-house gas emissions is at least about 10³, 10⁴, 10⁵, or 10⁶ CFU per seed or treated plant part. In certain embodiments, an effective amount of methanotroph provided in a treatment of a seed or plant part is at least about 10³, 10⁴, 10⁵, or 10⁶ CFU to about 10⁷, 10⁸, 10⁹, or 10¹⁰ CFU per seed or treated plant part. In certain embodiments, the effective amount of methanotroph provided in a treatment of a seed or plant part is an amount where the CFU per seed or treated plant part will exceed the number of CFU of any resident naturally occurring methanotroph strain by at least 5-, 10-, 100-, or 1000-fold. In certain embodiments, the effective amount of methanotroph provided in a treatment of a seed or plant part is an amount where the CFU per seed or treated plant part will exceed the number of CFU of any resident naturally occurring methanotroph by at least 2-, 3-, 5-, 8-, 10-, 20-, 50-, 100-, or 1000-fold. In certain embodiments where the treated plant is cultivated in a hydroponic system, populations of naturally occurring methanotroph or other soil microbes will be minimal.

For liquid compositions, the concentration of the methanotroph strain can be from about 1×10³ CFU/mL to about 1×10¹⁰ CFU/mL, from about 5×10³ CFU/mL to about 1×10¹⁰ CFU/mL, from about 1×10⁴ CFU/mL to about 1×10¹⁰ CFU/mL, from about 5×10⁴ CFU/mL to about 1×10¹⁰ CFU/mL, from about 1×10⁵ CFU/mL to about 1×10¹⁰ CFU/mL, from about 5×10⁵ CFU/mL to about 1×10¹⁰ CFU/mL, or from about 1×10⁶ CFU/mL to about 1×10¹⁰ CFU/mL.

For solid compositions, concentration of the methanotroph strain can be from about 1×10³ CFU/g to about 1×10¹⁰ CFU/g, from about 5×10³ CFU/g to about 1×10¹⁰ CFU/g, from about 1×10⁴ CFU/g to about 1×10¹⁰ CFU/g, from about 5×10⁴ CFU/g to about 1×10¹⁰ CFU/g, from about 1×10⁵ CFU/g to about 1×10¹⁰ CFU/g, from about 5×10⁵ CFU/g to about 1×10¹⁰ CFU/g, or from about 1×10⁶ CFU/g to about 1×10¹⁰ CFU/g.

Non-limiting examples of Methylobacterium strains that can be used in methods provided herein are disclosed in Table 1. Other Methylobacterium strains useful in certain methods provided herein include variants of the Methylobacterium strains disclosed in Table 1. Also of use are various combinations of two or more strains or variants of Methylobacterium strains disclosed in Table 1 for treatment of plants or parts thereof.

TABLE 1
Methylobacterium sp. strain

| | LGP/NLS | USDA ARS | |
|---|---|---|---|
| Deposit Identifier | NO. | NRRL No.¹ | Strain Taxonomy |
| Methylobacterium sp. #1 | LGP2000 | NRRL B-50929 | M. gregans |
| Methylobacterium sp. #2 | LGP2001 | NRRL B-50930 | M. radiotolerans |
| Methylobacterium sp. #3 | LGP2002 | NRRL B-50931 | M. radiotolerans |
| Methylobacterium sp. #4 | LGP2003 | NRRL B-50932 | M. extorquens |
| Methylobacterium sp. #5 | LGP2004 | NRRL B-50933 | M. populi |
| Methylobacterium sp. #6 | LGP2005 | NRRL B-50934 | M. extorquens |
| Methylobacterium sp. #7 | LGP2006 | NRRL B-50935 | M. extorquens |
| Methylobacterium sp. #8 | LGP2007 | NRRL B-50936 | M. salsuginis |
| Methylobacterium sp. #9 | LGP2008 | NRRL B-50937 | M. extorquens |
| Methylobacterium sp. #10 | LGP2009 | NRRL B-50938 | M. gregans |
| Methylobacterium sp. #11 | LGP2010 | NRRL B-50939 | M. populi |
| Methylobacterium sp. #12 | LGP2011 | NRRL B-50940 | M. gregans |
| Methylobacterium sp. #13 | LGP2012 | NRRL B-50941 | M. brachiatum |
| Methylobacterium sp. #14 | LGP2013 | NRRL B-50942 | M. extorquens |
| Methylobacterium sp. #15 | LGP2014 | NRRL B-67339 | M. gregans |
| Methylobacterium sp. #16 | LGP2015 | NRRL B-67340 | M. komagatae |
| Methylobacterium sp. #17 | LGP2016 | NRRL B-67341 | M. gregans |
| Methylobacterium sp. #18 | LGP2017 | NRRL B-67741 | M. radiotolerans |
| Methylobacterium sp. #19 | LGP2018 | NRRL B-67742 | M. komagatae |
| Methylobacterium sp. #20 | LGP2019 | NRRL B-67743 | M. gregans |
| Methylobacterium sp. #22 | NLS0497 | NRRL B-67925 | M. radiotolerans |
| Methylobacterium sp. #23 | NLS0693 | NRRL B-67926 | M. komagatae |
| Methylobacterium sp. #24 | NLS1179 | NRRL B-67929 | M. bullatum |
| Methylobacterium sp. #25 | LGP2167 | NRRL B-67927 | M. dankookense |
| Methylobacterium sp. #26 | LGP2020 | NRRL-B-67892 | M. radiotolerans |
| Methylobacterium sp. #28 | LGP2021 | NRRL-B-68032 | M. komagatae |
| Methylobacterium sp. #29 | LGP2022 | NRRL-B-68033 | M. radiotolerans |
| Methylobacterium sp. #30 | LGP2023 | NRRL-B-68034 | M. radiotolerans |
| Methylobacterium sp. #31 | LGP2028 | NRRL-B-68064 | M. radiotolerans |
| Methylobacterium sp #32 | LGP2029 | NRRL B-68065 | M. komagatae |
| Methylobacterium sp #33 | LGP2030 | NRRL B-68066 | M. radiotolerans |
| Methylobacterium sp #34 | LGP2031 | NRRL B-68067 | M. komagatae |
| Methylobacterium sp #35 | LGP2033 | NRRL B-68068 | M. radiotolerans |
| Methylobacterium sp #36 | LGP2034 | NRRL B-68069 | M. komagatae |
| Methylobacterium sp. #37 | NLS0737 | NRRL-B-68074 | M. extorquens |
| Methylobacterium sp. #38 | NLS0770 | NRRL-B-68075 | M. extorquens |
| Methylobacterium sp. #39 | NLS5278 | NRRL-B-68186 | M. radiotolerans |
| Methylobacterium sp. #40 | NLS5334 | NRRL-B-68187 | M. radiotolerans |
| Methylobacterium sp. #41 | NLS5480 | NRRL-B-68188 | M. radiotolerans |
| Methylobacterium sp. #42 | NLS5549 | NRRL-B-68189 | M. radiotolerans |
| Methylobacterium sp #43 | NLS0665 | NRRL-B-68194 | M. radiotolerans |
| Methylobacterium sp #44 | NLS0729 | NRRL-B-68195 | M. radiotolerans |
| Methylobacterium sp #45 | NLS0672 | NRRL-B-68196 | M. mesophilicum |
| Methylobacterium sp #46 | NLS0754 | NRRL-B-68197 | M. radiotolerans |
| Methylobacterium sp #47 | NLS0591 | NRRL-B-68215 | M. komagatae |
| Methylobacterium sp #48 | NLS0439 | NRRL-B-68216 | M. brachiatum |
| Methylobacterium sp #49 | NLS1310 | NRRL-B-68217 | M. radiotolerans |
| Methylobacterium sp #50 | NLS1312 | NRRL-B-68218 | M. radiotolerans |
| Methylobacterium sp #51 | NLS0049 | NRRL-B-68236 | M. goesingense |
| Methylobacterium sp #52 | NLS0612 | NRRL-B-68237 | M. radiotolerans |
| Methylobacterium sp #53 | NLS0706 | NRRL-B-68238 | M. komagatae |
| Methylobacterium sp #54 | NLS0725 | NRRL-B-68239 | M. komagatae |
| Methylomicrobium sp. #55 | NLS1501 | NRRL B-68261 | Methylomicrobium lacus |
| Methylosarcina sp. #56 | NLS1504 | NRRL B-68281 | Methylosarcina fibrata |
| Methylocystis sp. #57 | NLS1505 | NRRL B-68282 | Methylocystis hirsuta |
| Methylocystis sp. #58 | NLS1506 | NRRL B-68283 | Methylocystis hirsuta |
| Methylocystis sp. #59 | NLS1508 | NRRL B-68262 | Methylocystis hirsuta |
| Methylocystis sp. #60 | NLS1509 | NRRL B-68284 | Methylocystis hirsuta |
| Methylocystis sp. #61 | NLS1511 | NRRL B-68285 | Methylocystis hirsuta |
| Methylocystis sp. #62 | NLS1512 | NRRL B-68286 | Methylocystis hirsuta |
| Methylorubrum sp. #63 | NLS7725 | NRRL B-68260 | Methylorubrum populi |

¹Deposit number for strain deposited with the AGRICULTURAL RESEARCH SERVICE CULTURE COLLECTION (NRRL) of the National Center for Agricultural Utilization Research, Agricultural Research Service, U.S. Department of Agriculture, 1815 North University Street, Peoria, Illinois 61604 U.S.A. under the terms of the Budapest Treaty on the International Recognition of the Deposit of Microorganisms for the Purposes of Patent Procedure. Subject to 37 CFR §1.808(b), all restrictions imposed by the depositor on the availability to the public of the deposited material will be irrevocably removed upon the granting of any patent from this patent application.

Variants of a Methylobacterium or methanotroph isolate listed in Table 1 include isolates obtained therefrom by genetic transformation, mutagenesis and/or insertion of a heterologous sequence. In some embodiments, such variants are identified by the presence of chromosomal genomic DNA with at least 99%, 99.9, 99.8, 99.7, 99.6%, or 99.5% sequence identity to chromosomal genomic DNA of the strain from which it was derived. In certain embodiments of the methods provided herein, the Methylobacterium strain or methanotroph strain or strains used to treat a plant seed and/or a plant part are selected from the group consisting of LGP2000 (NRRL B-50929), LGP2001 (NRRL B-50930), LGP2002 (NRRL B-50931), LGP2003 (NRRL B-50932), LGP2004 (NRRL B-50933), LGP2005 (NRRL B-50934), LGP2006 (NRRL B-50935), LGP2007 (NRRL B-50936), LGP2008 (NRRL B-50937), LGP2009 (NRRL B-50938), LGP2010 (NRRL B-50939), LGP2011 (NRRL B-50940), LGP2012 (NRRL B-50941), LGP2013 (NRRL B-50942), LGP2014 (NRRL B-67339), LGP2015 (NRRL B-67340), LGP2016 (NRRL B-67341), LGP2017 (NRRL B-67741), LGP2018 (NRRL B-67742), LGP2019 (NRRL B-67743), NLS0497 (NRRL B-67925), NLS0693 (NRRL B-67926), NLS1179 (NRRL B-67929), LGP2167 (NRRL B-67927), LGP2020 (NRRL B-67892), LGP2021 (NRRL-B-68032), LGP2022 (NRRL-B-68033), LGP2023 (NRRL-B-68034), LGP2028 (NRRL B-68064), LGP2029 (NRRL B-68065), LGP2030 (NRRL B-68066), LGP2031 (NRRL B-68067), LGP2033 (NRRL B-68068), LGP2034 (NRRL B-68069), NLS0665 (NRRL-B-68194), NLS0729 (NRRL-B-68195), NLS0672 (NRRL-B-68196), NLS0754 (NRRL-B-68197), NLS0049 (NRRL-B-68236), NLS0591 (NRRL-B-68215), NLS0439 (NRRL-B-68216), NLS1310 (NRRL-B-68217), NLS1312 (NRRL-B-68218), NLS0612 (NRRL-B-68237), NLS0706 (NRRL-B-68238), NLS0725 (NRRL-B-68239), NLS7725, NLS0770 (NRRL-B-68075), NLS0737 (NRRL-B-68074), NLS5278 (NRRL-B-68186), NLS5334 (NRRL-B-68187), NLS5480 (NRRL-B-68188), NLS5549 (NRRL-B-68189), NLS1501, NLS1504, NLS1505, NLS1506, NLS1508, NLS1509, NLS1511, NLS1512, variants thereof, or any combination thereof. In certain embodiments, one or more of the Methylobacterium strains used in the methods can comprise total genomic DNA (chromosomal and plasmid DNA) or average nucleotide identity (ANI) with at least 99%, 99.9, 99.8, 99.7, 99.6%, or 99.5% sequence identity or ANI to total genomic DNA of LGP2000 (NRRL B-50929), LGP2001 (NRRL B-50930), LGP2002 (NRRL B-50931), LGP2003 (NRRL B-50932), LGP2004 (NRRL B-50933), LGP2005 (NRRL B-50934), LGP2006 (NRRL B-50935), LGP2007 (NRRL B-50936), LGP2008 (NRRL B-50937), LGP2009 (NRRL B-50938), LGP2010 (NRRL B-50939), LGP2011 (NRRL B-50940), LGP2012 (NRRL B-50941), LGP2013 (NRRL B-50942), LGP2014 (NRRL B-67339), LGP2015 (NRRL B-67340), LGP2016 (NRRL B-67341), LGP2017 (NRRL B-67741), LGP2018 (NRRL B-67742), LGP2019 (NRRL B-67743), NLS0497 (NRRL B-67925), NLS0693 (NRRL B-67926), NLS1179 (NRRL B-67929), LGP2167 (NRRL B-67927), LGP2020 (NRRL B-67892), LGP2021 (NRRL-B-68032), LGP2022 (NRRL-B-68033), LGP2023 (NRRL-B-68034), LGP2028 (NRRL B-68064), LGP2029 (NRRL B-68065), LGP2030 (NRRL B-68066), LGP2031 (NRRL B-68067), LGP2033 (NRRL B-68068), LGP2034 (NRRL B-68069), NLS0665 (NRRL-B-68194), NLS0729 (NRRL-B-68195), NLS0672 (NRRL-B-68196), NLS0754 (NRRL-B-68197), NLS0049 (NRRL-B-68236), NLS0591 (NRRL-B-68215), NLS0439 (NRRL-B-68216), NLS1310 (NRRL-B-68217), NLS1312 (NRRL-B-68218), NLS0612 (NRRL-B-68237), NLS0706 (NRRL B-68238), NLS0725 (NRRL-B-68239), NLS0770 (NRRL-B-68075), NLS0737 (NRRL-B-68074), NLS5278 (NRRL-B-68186), NLS5334 (NRRL-B-68187), NLS5480 (NRRL-B-68188), or NLS5549 (NRRL-B-68189). In certain embodiments, one or more of the methanotroph strains used in the methods can comprise total genomic DNA (chromosomal and plasmid DNA) or average nucleotide identity (ANI) with at least 99%, 99.9, 99.8, 99.7, 99.6%, or 99.5% sequence identity or ANI to total genomic DNA of NLS1501, NLS1504, NLS1505, NLS1506, NLS1508, NLS1509, NLS1511, or NLS1512. In certain embodiments, the percent ANI can be determined as disclosed by Konstantinidis et al., 2006. In certain embodiments of the methods provided herein, a methanotroph strain or strains used to treat soil, water, a plant, a seed and/or a plant part is NLS1501, NLS1504, NLS1505, NLS1506, NLS1508, NLS1509, NLS1511, or NLS1512.

In certain embodiments of the methods provided herein, plants, plant seeds and/or plant parts are treated with both a methanotroph strain and at least one additional component. In some embodiments an additional component can be an additional active ingredient, for example, a pesticide or a second biological. In certain embodiments, the pesticide can be an insecticide, a fungicide, an herbicide, a nematicide or other biocide. The second biological could be a strain that improves yield or controls an insect, pest, fungi, weed, or nematode. In some embodiments, a second biological is an additional methanotroph strain. In some embodiments, a second biological is a Methylobacterium strain. In some embodiments, an additional Methylobacterium strain in the methods and compositions provided herein is selected from the Methylobacterium strains listed in Table 1.

Non-limiting examples of insecticides and nematicides include carbamates, diamides, macrocyclic lactones, neonicotinoids, organophosphates, phenylpyrazoles, pyrethrins, spinosyns, synthetic pyrethroids, tetronic and tetramic acids. In particular embodiments insecticides and nematicides include abamectin, aldicarb, aldoxycarb, bifenthrin, carbofuran, chlorantraniliprole, clothianidin, cyfluthrin, cyhalothrin, cypermethrin, deltamethrin, dinotefuran, emamectin, ethiprole, fenamiphos, fipronil, flubendiamide, fosthiazate, imidacloprid, ivermectin, lambda-cyhalothrin, milbemectin, nitenpyram, oxamyl, permethrin, tioxazafen, spinetoram, spinosad, spirodiclofen, spirotetramat, tefluthrin, thiacloprid, thiamethoxam, and thiodicarb.

Non-limiting examples of useful fungicides include aromatic hydrocarbons, benzimidazoles, benzothiadiazole, carboxamides, carboxylic acid amides, morpholines, phenylamides, phosphonates, quinone outside inhibitors (e.g. strobilurins), thiazolidines, thiophanates, thiophene carboxamides, and triazoles. Particular examples of fungicides include acibenzolar-S-methyl, azoxystrobin, benalaxyl, bixafen, boscalid, carbendazim, cyproconazole, dimethomorph, epoxiconazole, fluopyram, fluoxastrobin, flutianil, flutolanil, fluxapyroxad, fosetyl-Al, ipconazole, isopyrazam, kresoxim-methyl, mefenoxam, metalaxyl, metconazole, myclobutanil, orysastrobin, penflufen, penthiopyrad, picoxystrobin, propiconazole, prothioconazole, pyraclostrobin, sedaxane, silthiofam, tebuconazole, thifluzamide, thiophanate, tolclofos-methyl, trifloxystrobin, and triticonazole. Non-limiting examples of other biocides, include isothiazolinones, for example 1,2 Benzothiazolin-3-one (BIT), 5-Chloro-2-methyl-4-isothiazolin-3-one (CIT), 2-Methyl-4-isothiazolin-3-one (MIT), octylisothiazolinone (OIT), dichlorooctylisothiazolinone (DCOIT), and butylbenzisothiazolinone (BBIT); 2-Bromo-2-nitro-propane-1,3-diol (Bronopol), 5-bromo-5-nitro-1,3-dioxane (Bronidox), Tris(hydroxymethyl)nitromethane, 2,2-Dibromo-3-nitrilopropionamide (DBNPA), and alkyl dimethyl benzyl ammonium chlorides.

Non-limiting examples of herbicides include ACCase inhibitors, acetanilides, AHAS inhibitors, carotenoid biosynthesis inhibitors, EPSPS inhibitors, glutamine synthetase inhibitors, PPO inhibitors, PS II inhibitors, and synthetic auxins, Particular examples of herbicides include acetochlor, clethodim, dicamba, flumioxazin, fomesafen, glyphosate, glufosinate, mesotrione, quizalofop, saflufenacil, sulcotrione, and 2,4-D.

In some embodiments, the composition or method disclosed herein may comprise a methanotroph strain and an additional active ingredient selected from the group consisting of clothianidin, ipconazole, imidacloprid, metalaxyl, mefenoxam, tioxazafen, azoxystrobin, thiomethoxam, fluopyram, prothioconazole, pyraclostrobin, and sedaxane.

In some embodiments, the composition or method disclosed herein may comprise an additional active ingredient, which may be a second biological. The second biological could be a biological control agent, other beneficial microorganisms, microbial extracts, plant extracts, yeast extracts, vegetal chitosan, natural products, plant growth activators or plant defense agent. Non-limiting examples of the second biological could include bacteria, fungi, beneficial nematodes, and viruses. In certain embodiments, the second biological can be a Methylobacterium. In certain embodiments, the second biological is a Methylobacterium listed in Table 1. In certain embodiments, the second biological can be a Methylobacterium selected from M. gregans, M. radiotolerans, M. extorquens, M. populi, M salsuginis, M. brachiatum, and M. komagatae.

In certain embodiments, the second biological can be a bacterium of the genus Actinomycetes, Agrobacterium, Arthrobacter, Alcaligenes, Aureobacterium, Azobacter, Azorhizobium, Azospirillum, Azotobacter, Beijerinckia, Bacillus, Brevibacillus, Burkholderia, Chromobacterium, Clostridium, Clavibacter, Comomonas, Corynebacterium, Curtobacterium, Enterobacter, Flavobacterium, Gluconacetobacter, Gluconobacter, Herbaspirillum, Hydrogenophaga, Klebsiella, Luteibacter, Lysinibacillus, Mesorhizobium, Methylobacterium, Microbacterium, Ochrobactrum, Paenibacillus, Pantoea, Pasteuria, Phingobacterium, Photorhabdus, Phyllobacterium, Pseudomonas, Rhizobium, Rhodococcus, Bradyrhizobium, Serratia, Sinorhizobium, Sphingomonas, Streptomyces, Stenotrophomonas, Variovorax, Xanthomonas and Xenorhabdus. In particular embodiments the bacteria is selected from the group consisting of Bacillus amyloliquefaciens, Bacillus cereus, Bacillus firmus, Bacillus, lichenformis, Bacillus pumilus, Bacillus sphaericus, Bacillus subtilis, Bacillus thuringiensis, Chromobacterium suttsuga, Pasteuria penetrans, Pasteuria usage, and Pseudomona fluorescens.

In certain embodiments the second biological can be a fungus of the genus Acremonium, Alternaria, Ampelomyces, Aspergillus, Aureobasidium, Beauveria, Botryosphaeria, Cladosporium, Cochliobolus, Colletotrichum, Coniothyrium, Embellisia, Epicoccum, Fusarium, Gigaspora, Gliocladium, Glomus, Laccaria, Metarhisium, Muscodor, Nigrospora, Paecilonyces, Paraglomus, Penicillium, Phoma, Pisolithus, Podospora, Rhizopogon, Scleroderma, Trichoderma, Typhula, Ulocladium, and Verticillium. In particular embodiments, the fungus is Beauveria bassiana, Coniothyrium minitans, Gliocladium vixens, Muscodor albus, Paecilomyces lilacinus, or Trichoderma polysporum.

In certain embodiments, compositions comprise multiple additional biological ingredients, including consortia comprising combinations of any of the above bacterial or fungal genera or species. In further embodiments the second biological can be a biostimulant, including but not limited to seaweed extract or humates, plant growth activators or plant defense agents including, but not limited to harpin, Reynoutria sachalinensis, jasmonate, lipochitooligosaccharides, and isoflavones.

In further embodiments, the second biological can include, but are not limited to, various Bacillus sp., Pseudomonas sp., Coniothyrium sp., Pantoea sp., Streptomyces sp., and Trichoderma sp. Microbial biopesticides can be a bacterium, fungus, virus, or protozoan. Particularly useful biopesticidal microorganisms include various Bacillus subtilis, Bacillus thuringiensis, Bacillus pumilis, Pseudomonas syringae, Trichoderma harzianum, Trichoderma virens, and Streptomyces lydicus strains. Other microorganisms that are added can be genetically engineered or wild-type isolates that are available as pure cultures. In certain embodiments, it is anticipated that the second biological can be provided in the composition in the form of a spore.

Fields, plants or harvested plant parts having mitigated methane in comparison to a control field, plant, or plant part are provided, as are methods for obtaining and using such plants and plant parts. In certain embodiments, the content of at least mitigated methane is decreased by at least about 0.1%, 5%, 1%, or 2% to about 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%.

### Deposit Information

Samples of the following Methylobacterium sp. strains have been deposited with the AGRICULTURAL RESEARCH SERVICE CULTURE COLLECTION (NRRL) of the National Center for Agricultural Utilization Research, Agricultural Research Service, U.S. Department of Agriculture, 1815 North University Street, Peoria, Illinois 61604 U.S.A. under the terms of the Budapest Treaty on the International Recognition of the Deposit of Microorganisms for the Purposes of Patent Procedure. Methylobacterium sp. NRRL B-50929, NRRL B-50930, NRRL B-50931, NRRL B-50932, NRRL B-50933, NRRL B-50934, NRRL B-50935, NRRL B-50936, NRRL B-50937, NRRL B-50938, NRRL B-50939, NRRL B-50940, NRRL B-50941 and NRRL B-50942 were deposited with NRRL on Mar. 12, 2014. Methylobacterium sp. NRRL B-67339, NRRL B-67340, and NRRL B-67341 were deposited with NRRL on Nov. 18, 2016. Methylobacterium sp. NRRL B-67741, NRRL B-67742, NRRL B-67743 were deposited with NRRL on Dec. 20, 2018. Methylobacterium sp. NRRL B-67892 was deposited with NRRL on Nov. 26, 2019. Methylobacterium sp. NRRL B-67925, NRRL B-67926 and NRRL B-67927 were deposited with NRRL on Feb. 21, 2020. Methylobacterium sp. NRRL B-67929 was deposited with NRRL on Mar. 3, 2020. Methylobacterium sp. NRRL B-68032, NRRL B-68033 and NRRL B-68034 were deposited with NRRL on May 20, 2021. Methylobacterium sp. NRRL B-68064, NRRL B-68065, NRRL B-68066, NRRL B-68067, NRRL B-68068, and NRRL B-68069 were deposited with NRRL on Sep. 9, 2021. Methylobacterium sp. NRRL B-68074 and NRRL B-68075 were deposited with NRRL on Oct. 6, 2021. Methylobacterium sp. NRRL B-68186, NRRL B-68187, NRRL B-68188 and NRRL B-68189 were deposited with NRRL on Aug. 3, 2022. Methylobacterium sp. NRRL B-68194, NRRL B-68195, NRRL B-68196, and NRRL B-68197 were deposited with NRRL on Aug. 30, 2022. Methylobacterium sp. NRRL B-68215, NRRL B-68216, NRRL B-68217, and NRRL B-68218 were deposited with NRRL on Nov. 2, 2022. Methylobacterium sp. NRRL B-68236, NRRL B-68237, NRRL B-68238, and NRRL B-68239 were deposited with NRRL on Nov. 23, 2022. Methylomicrobium sp. NRRL B-68261 and Methylocystis sp. NRRL B-68262 were deposited with NRRL on Feb. 14, 2023. Methylorubrum sp. NRRL B-68260 was deposited with NRRL on Mar. 9, 2023. Methylosarcina sp. NRRL B-68281, and Methylocystis sp. NRRL B-68282, NRRL B-68283, NRRL B-68284, NRRL B-68285, and NRRL B-68286 were deposited with NRRL on Jun. 7, 2023.

Additionally, the following embodiments are included in the disclosure.

Embodiment 1. A method for mitigating methane gas in an agricultural field that comprises: (a) applying a composition to a soil, field, plant, plant part or seed, wherein the composition comprises at least one methanotroph selected from the group consisting of a Type II methanotroph comprising a pMMO2 protein, and Type I methanotroph, (b) growing the methanotroph whereby the methanotroph uses methane as a carbon source; wherein use of the methane as the carbon source oxidizes methane or reduces methane emissions.

Embodiment 2. The method of Embodiment 1, wherein the Type II methanotroph is a Methylocystis sp.

Embodiment 3. The method of Embodiment 2, wherein the Methylocystis sp. is Methylocystis hirsuta isolate.

Embodiment 4. The method of Embodiment 3, wherein the Methylocystis hirsuta isolate comprises PmoA2, PmoB2 and PmoC2 proteins with sequences having at least 97% identity to SEQ ID NOS:76-78.

Embodiment 5. The method of Embodiment 3, wherein said Methylocystis hirsuta isolate is selected from the group consisting of NLS1505, NLS1506, NLS1508, NLS1509, NLS1511, NLS1512, and variants thereof.

Embodiment 6. The method of any one of Embodiments 1 to 5, wherein the Type I methanotroph is a Methylomicrobium or Methylosarcina species.

Embodiment 7. The method of Embodiment 6 wherein said Type I methanotroph comprises NLS1501, NLS1504, or a variant thereof.

Embodiment 8. The method of any one of Embodiments 1 to 7, wherein the composition is applied to an irrigated field or flooded field.

Embodiment 9. The method of any one of Embodiments 1 to 8, wherein said plant, plant part, or seed is rice.

Embodiment 10. The method of any one of Embodiments 1 to 9, where the composition is applied to a flooded or irrigated rice field.

Embodiment 11. The method of any one of Embodiments 1 to 10, wherein said composition further comprises at least one additional component selected from the group consisting of an additional active ingredient, an agriculturally acceptable adjuvant, and an agriculturally acceptable excipient.

Embodiment 12. A composition comprising a fermentation product comprising a methanotroph strain, wherein said fermentation product is essentially free of contaminating microorganisms, and wherein methanotroph strain comprises NLS1501, NLS1504, NLS1505, NLS1506, NLS1508, NLS1509, NLS1511, NLS1512, or a variant thereof.

Embodiment 13. The composition of Embodiment 12, wherein said composition further comprises at least one additional component selected from the group consisting of an additional active ingredient, an agriculturally acceptable adjuvant, and an agriculturally acceptable excipient.

Embodiment 14. The composition of Embodiment 12, wherein said composition further comprises a methylotroph.

Embodiment 15. The composition of Embodiment 14 wherein said methylotroph is a Methylobacterium strain.

Embodiment 16. The composition of Embodiment 15 wherein the Methylobacterium comprises LGP2000 (NRRL B-50929), LGP2001 (NRRL B-50930), LGP2002 (NRRL B-50931), LGP2003 (NRRL B-50932), LGP2004 (NRRL B-50933), LGP2005 (NRRL B-50934), LGP2006 (NRRL B-50935), LGP2007 (NRRL B-50936), LGP2008 (NRRL B-50937), LGP2009 (NRRL B-50938), LGP2010 (NRRL B-50939), LGP2011 (NRRL B-50940), LGP2012 (NRRL B-50941), LGP2013 (NRRL B-50942), LGP2014 (NRRL B-67339), LGP2015 (NRRL B-67340), LGP2016 (NRRL B-67341), LGP2017 (NRRL B-67741), LGP2018 (NRRL B-67742), LGP2019 (NRRL B-67743), NLS0497 (NRRL B-67925), NLS0693 (NRRL B-67926), NLS1179 (NRRL B-67929), LGP2167 (NRRL B-67927), LGP2020 (NRRL-B-67892), LGP2021 (NRRL-B-68032), LGP2022 (NRRL-B-68033), LGP2023 (NRRL-B-68034), LGP2028 (NRRL B-68064), LGP2029 (NRRL B-68065), LGP2030 (NRRL B-68066), LGP2031 (NRRL B-68067), LGP2033 (NRRL B-68068), LGP2034 (NRRL B-68069), NLS0665 (NRRL B-68194), NLS0754 (NRRL B-68197), NLS0672 (NRRL B-68196), NLS0729 (NRRL B-68195), NLS0049 (NRRL-B-68236), NLS0591 (NRRL-B-68215), NLS0439 (NRRL-B-68216), NLS1310 (NRRL-B-68217), NLS1312 (NRRL-B-68218), NLS0612 (NRRL-B-68237), NLS0706 (NRRL B-68238), NLS0725 (NRRL-B-68239), NLS0770 (NRRL B-68075), NLS0737 (NRRL B-68074), NLS5278 (NRRL-B-68186), NLS5334 (NRRL-B-68187), NLS5480 (NRRL-B-68188), or NLS5549 (NRRL-B-68189), NLS7725 or a variant thereof, or a combination thereof.

Embodiment 17. The composition of Embodiment 15 wherein the Methylobacterium comprises LGP2002 (NRRL B-50931), LGP2003 (NRRL B-50932), LGP2004 (NRRL B-50933), LGP2009 (NRRL B-50938), LGP2015 (NRRL B-67340), LGP2016 (NRRL B-67341), LGP2017 (NRRL B-67741), LGP2019 (NRRL B-67743), NLS0693 (NRRL B-67926), or LGP2020 (NRRL-B-67892), NLS7725 or a variant thereof, or a combination thereof.

Embodiment 18. A plant, plant part or seed at least partially coated with the composition of any one of Embodiments 12 to 17.

Embodiment 19. A method for mitigating methanol that comprises: (a) treating a rice field with a composition comprising at least one methanotroph selected from the group consisting of a Type II methanotroph comprising a pMMO2 protein, and Type I methanotroph, wherein said Type I methanotroph is a Methylomicrobium sp. or a Methylosarcina sp.; and (b) growing the methanotroph in the field thereby mitigating methane.

Embodiment 20. The method of Embodiment 19, wherein said composition comprises a methanotroph isolate comprising NLS1501, NLS1504, NLS1505, NLS1506, NLS1508, NLS1509, NLS1511, NLS1512, or a combination thereof.

Embodiment 21. The method of Embodiment 20, wherein said methanotroph is present in at a concentration of greater than 1×10³ colony forming units (CFU) per milliliter.

Embodiment 22. An isolated methanotroph selected from NLS1501, NLS1504, NLS1505, NLS1506, NLS1508, NLS1509, NLS1511, or NLS1512.

Embodiment 23. A method for selecting a methanotroph isolate capable of utilizing methane as a food source, wherein said method comprises: a) detecting in the genome of a methanotroph isolate, one or more genetic elements, wherein said genetic element comprises a particulate methane monooxygenase; and b) treating a field, water, plant, plant part or seed with said methanotroph isolate, and measuring green-house gas emissions.

Embodiment 24. The method of Embodiment 23 wherein said plant is a rice plant.

Embodiment 25. The method of any one of Embodiments 1 to 11 or 19 to 24 wherein said composition further comprises a methylotroph and wherein said methylotroph is a Methylobacterium strain.

Embodiment 26. The method of Embodiment 25 wherein said Methylobacterium strain enhances growth, yield, nutrient update, or nitrogen use efficiency, and/or oxidizes methane

Embodiment 27. The method of Embodiment 26 wherein the Methylobacterium comprises LGP2000 (NRRL B-50929), LGP2001 (NRRL B-50930), LGP2002 (NRRL B-50931), LGP2003 (NRRL B-50932), LGP2004 (NRRL B-50933), LGP2005 (NRRL B-50934), LGP2006 (NRRL B-50935), LGP2007 (NRRL B-50936), LGP2008 (NRRL B-50937), LGP2009 (NRRL B-50938), LGP2010 (NRRL B-50939), LGP2011 (NRRL B-50940), LGP2012 (NRRL B-50941), LGP2013 (NRRL B-50942), LGP2014 (NRRL B-67339), LGP2015 (NRRL B-67340), LGP2016 (NRRL B-67341), LGP2017 (NRRL B-67741), LGP2018 (NRRL B-67742), LGP2019 (NRRL B-67743), NLS0497 (NRRL B-67925), NLS0693 (NRRL B-67926), NLS1179 (NRRL B-67929), LGP2167 (NRRL B-67927), LGP2020 (NRRL-B-67892), LGP2021 (NRRL-B-68032), LGP2022 (NRRL-B-68033), LGP2023 (NRRL-B-68034), LGP2028 (NRRL B-68064), LGP2029 (NRRL B-68065), LGP2030 (NRRL B-68066), LGP2031 (NRRL B-68067), LGP2033 (NRRL B-68068), LGP2034 (NRRL B-68069), NLS0665 (NRRL B-68194), NLS0754 (NRRL B-68197), NLS0672 (NRRL B-68196), NLS0729 (NRRL B-68195), NLS0049 (NRRL-B-68236), NLS0591 (NRRL-B-68215), NLS0439 (NRRL-B-68216), NLS1310 (NRRL-B-68217), NLS1312 (NRRL-B-68218), NLS0612 (NRRL-B-68237), NLS0706 (NRRL B-68238), NLS0725 (NRRL-B-68239), NLS0770 (NRRL B-68075), NLS0737 (NRRL B-68074), NLS5278 (NRRL-B-68186), NLS5334 (NRRL-B-68187), NLS5480 (NRRL-B-68188), NLS5549 (NRRL-B-68189), or NLS7725, or a variant thereof, or a combination thereof.

Embodiment 28. The method of Embodiment 27 wherein the Methylobacterium comprises LGP2002 (NRRL B-50931), LGP2003 (NRRL B-50932), LGP2004 (NRRL B-50933), LGP2009 (NRRL B-50938), LGP2015 (NRRL B-67340), LGP2016 (NRRL B-67341), LGP2017 (NRRL B-67741), LGP2019 (NRRL B-67743), NLS0693 (NRRL B-67926), or LGP2020 (NRRL-B-67892), NLS7725 or a variant thereof, or a combination thereof.

Embodiment 29. A method for mitigating methane that comprises: (a) treating pasture, wasteland, a landfill or waste with a composition comprising at least one methanotroph isolate; and (b) growing the methanotroph in the pasture, wasteland, a landfill or waste thereby mitigating methane.

Embodiment 30. A method for mitigating methane from livestock feed that comprises: (a) treating land with a composition comprising at least one methanotroph isolate; and (b) growing the methanotroph in the land thereby mitigating methane from livestock feed.

Embodiment 31. A method for reducing methane emissions from a methane emitting source, the method comprising applying a composition comprising at least one methanotroph isolate to the methane emitting source.

Embodiment 32. A method for reducing methane concentration in a methane-containing media (e.g., manure or livestock waste) or fluid (e.g., any methane-containing gas or liquid such as methane-contaminated groundwater), the method comprising applying a composition comprising at least one methanotroph isolate to the media or fluid.

Embodiment 33. A method for reducing methane emissions (e.g., in a landfill), the method comprising applying a first coating of a composition comprising at least one methanotroph isolate to a first layer of material (e.g., overburden/soil or waste);
at least partially covering the first layer and first coating with a second layer of material (e.g., overburden/soil or additional waste); applying a second coating of the composition comprising the at least one methanotroph isolate to a second layer; and
growing the methanotroph.

Embodiment 34. The method of any one of Embodiments 29 to 33, wherein said composition further comprises a methylotroph.

Embodiment 35. The composition of any one of Embodiments 29 to 33 wherein said methanotroph comprises NLS1501, NLS1504, NLS1505, NLS1506, NLS1508, NLS1509, NLS1511, NLS1512, or a combination thereof.

Embodiment 36. The method of Embodiments 29 to 33 wherein said methanotroph comprises NLS1501, NLS1504, NLS1505, NLS1506, NLS1508, NLS1509, NLS1511, NLS1512, or a combination thereof, and said methylotroph is a Methylobacterium strain.

Embodiment 37. The method of Embodiment 36 wherein the Methylobacterium comprises LGP2000 (NRRL B-50929), LGP2001 (NRRL B-50930), LGP2002 (NRRL B-50931), LGP2003 (NRRL B-50932), LGP2004 (NRRL B-50933), LGP2005 (NRRL B-50934), LGP2006 (NRRL B-50935), LGP2007 (NRRL B-50936), LGP2008 (NRRL B-50937), LGP2009 (NRRL B-50938), LGP2010 (NRRL B-50939), LGP2011 (NRRL B-50940), LGP2012 (NRRL B-50941), LGP2013 (NRRL B-50942), LGP2014 (NRRL B-67339), LGP2015 (NRRL B-67340), LGP2016 (NRRL B-67341), LGP2017 (NRRL B-67741), LGP2018 (NRRL B-67742), LGP2019 (NRRL B-67743), NLS0497 (NRRL B-67925), NLS0693 (NRRL B-67926), NLS1179 (NRRL B-67929), LGP2167 (NRRL B-67927), LGP2020 (NRRL-B-67892), LGP2021 (NRRL-B-68032), LGP2022 (NRRL-B-68033), LGP2023 (NRRL-B-68034), LGP2028 (NRRL B-68064), LGP2029 (NRRL B-68065), LGP2030 (NRRL B-68066), LGP2031 (NRRL B-68067), LGP2033 (NRRL B-68068), LGP2034 (NRRL B-68069), NLS0665 (NRRL B-68194), NLS0754 (NRRL B-68197), NLS0672 (NRRL B-68196), NLS0729 (NRRL B-68195), NLS0049 (NRRL-B-68236), NLS0591 (NRRL-B-68215), NLS0439 (NRRL-B-68216), NLS1310 (NRRL-B-68217), NLS1312 (NRRL-B-68218), NLS0612 (NRRL-B-68237), NLS0706 (NRRL B-68238), NLS0725 (NRRL-B-68239), NLS0770 (NRRL B-68075), NLS0737 (NRRL B-68074), NLS5278 (NRRL-B-68186), NLS5334 (NRRL-B-68187), NLS5480 (NRRL-B-68188), NLS5549 (NRRL-B-68189), or NLS7725 or a variant thereof, or a combination thereof.

Embodiment 38. The method of Embodiment 37 wherein the Methylobacterium comprises LGP2002 (NRRL B-50931), LGP2003 (NRRL B-50932), LGP2004 (NRRL B-50933), LGP2009 (NRRL B-50938), LGP2015 (NRRL B-67340), LGP2016 (NRRL B-67341), LGP2017 (NRRL B-67741), LGP2019 (NRRL B-67743), NLS0693 (NRRL B-67926), or LGP2020 (NRRL-B-67892), NLS7725, or a variant thereof, or a combination thereof 0.39. The method of claim 35 wherein said composition further comprises a methylotroph.

Embodiment 40. A recombinant construct for expression of a pMMO component protein or a modification thereof, wherein said construct comprises a genetic element encoding SEQ ID NO: 77-96 or a modification thereof.

Embodiment 41. A method for identifying combinations of methanotroph and methyltroph bacterial strains useful for mitigating methane gas, wherein said method comprises a) isolating methanotroph and methylotroph bacterial strains, and b) treating a field, water, plant, plant part or seed with said methanotroph and methylotroph strains, 3) measuring green-house gas emissions and 4) identifying treatments resulting in decreased methane levels in said emissions as compared to methane levels resulting from a control treatment.

Embodiment 42. The method of Embodiment 41 wherein said control treatment comprises application of a methanotroph in the absence of said methylotroph.

Embodiment 43. The method of Embodiment 41 further comprising the step of measuring the biomass and yield of said treated plant or a plant grown in said treated field.

Embodiment 44. The method of Embodiment 41 wherein said treated plants are grown in a low methane environment.

Embodiment 45. The method of Embodiment 43 wherein said treated plants are grown in a high methane environment.

Embodiment 46. A method for increasing harvested yield of a plant, wherein said method comprises applying a composition to a soil, field, plant, plant part or seed, wherein the composition comprises at least one methanotroph.

Embodiment 47. The method of Embodiment 46, further comprising the steps of growing the plant to maturity and harvesting seed or other harvestable plant product from the mature plant.

Embodiment 48. The method of Embodiment 46 or 47 wherein said methanotroph is a methanotrophic microbe selected from the group consisting of a Type II methanotroph comprising a pMMO2 protein, and Type I methanotroph.

Embodiment 49. The method of Embodiment 48, wherein the Type II methanotroph is a Methylocystis sp.

Embodiment 50. The method of Embodiment 49, wherein the Methylocystis sp. is Methylocystis hirsuta.

Embodiment 51. The method of Embodiment 50, wherein the Methylocystis hirsuta isolate comprises PmoA2, PmoB2 and PmoC2 proteins with sequences having at least 97% identity to SEQ ID NOS:76-78.

Embodiment 52. The method of Embodiment 51, wherein said Methylocystis hirsuta isolate is selected from the group consisting of NLS1505, NLS1506, NLS1508, NLS1509, NLS1511, NLS1512, and variants thereof.

Embodiment 53. The method of Embodiment 48, wherein the Type I methanotroph is a Methylomicrobium or Methylosarcina species.

Embodiment 54. The method of Embodiment 53 wherein said Type I methanotroph comprises NLS1501, NLS1504, or a variant thereof.

Embodiment 55. The method of Embodiment 46 or 47 wherein said plant is a field crop plant.

Embodiment 56. The method of Embodiment 55 wherein said field crop is selected from the group consisting of rice, wheat, corn, soybean, peanut, barley, alfalfa, millet, sorghum, oat, and rye.

Embodiment 57. The method of Embodiment 46 or 47 wherein said plant is an herb, Cannabis, ornamental or turfgrass plant.

Embodiment 58. The method of any one of Embodiments 46 to 55 wherein application of said methanotroph mitigates methane gas associated with growth of said plant.

Embodiment 59. The method of any one of Embodiment 58, wherein the composition is applied to an irrigated field or flooded field.

Embodiment 60. The method of Embodiment 59, wherein said plant, plant part, or seed is rice.

Embodiment 61. The method of Embodiment 60, where the composition is applied to a flooded or irrigated rice field.

Embodiment 62. The method of any one of Embodiments 58 to 61, wherein said composition further comprises a methylotroph.

Embodiment 63. The method of Embodiment 62, wherein said methylotroph is a Methylobacterium strain.

Embodiment 64. The method of Embodiment 63 wherein the Methylobacterium comprises LGP2000 (NRRL B-50929), LGP2001 (NRRL B-50930), LGP2002 (NRRL B-50931), LGP2003 (NRRL B-50932), LGP2004 (NRRL B-50933), LGP2005 (NRRL B-50934), LGP2006 (NRRL B-50935), LGP2007 (NRRL B-50936), LGP2008 (NRRL B-50937), LGP2009 (NRRL B-50938), LGP2010 (NRRL B-50939), LGP2011 (NRRL B-50940), LGP2012 (NRRL B-50941), LGP2013 (NRRL B-50942), LGP2014 (NRRL B-67339), LGP2015 (NRRL B-67340), LGP2016 (NRRL B-67341), LGP2017 (NRRL B-67741), LGP2018 (NRRL B-67742), LGP2019 (NRRL B-67743), NLS0497 (NRRL B-67925), NLS0693 (NRRL B-67926), NLS1179 (NRRL B-67929), LGP2167 (NRRL B-67927), LGP2020 (NRRL-B-67892), LGP2021 (NRRL-B-68032), LGP2022 (NRRL-B-68033), LGP2023 (NRRL-B-68034), LGP2028 (NRRL B-68064), LGP2029 (NRRL B-68065), LGP2030 (NRRL B-68066), LGP2031 (NRRL B-68067), LGP2033 (NRRL B-68068), LGP2034 (NRRL B-68069), NLS0665 (NRRL B-68194), NLS0754 (NRRL B-68197), NLS0672 (NRRL B-68196), NLS0729 (NRRL B-68195), NLS0049 (NRRL-B-68236), NLS0591 (NRRL-B-68215), NLS0439 (NRRL-B-68216), NLS1310 (NRRL-B-68217), NLS1312 (NRRL-B-68218), NLS0612 (NRRL-B-68237), NLS0706 (NRRL B-68238), NLS0725 (NRRL-B-68239), NLS0770 (NRRL B-68075), NLS0737 (NRRL B-68074), NLS5278 (NRRL-B-68186), NLS5334 (NRRL-B-68187), NLS5480 (NRRL-B-68188), NLS5549 (NRRL-B-68189), or NLS7725 (NRRL B-68260), or a variant thereof, or a combination thereof.

Embodiment 65. The method of Embodiment 64 wherein the Methylobacterium comprises LGP2002 (NRRL B-50931), LGP2003 (NRRL B-50932), LGP2004 (NRRL B-50933), LGP2009 (NRRL B-50938), LGP2015 (NRRL B-67340), LGP2016 (NRRL B-67341), LGP2017 (NRRL B-67741), LGP2018 (NRRL B-67742), LGP2019 (NRRL B-67743), NLS0693 (NRRL B-67926), LGP2020 (NRRL-B-67892), NLS0754 (NRRL B-68197), NLS7725 (NRRL B-68260), or a variant thereof, or a combination thereof.

Embodiment 66. The method of any one of Embodiments 46 to 51, wherein said composition further comprises at least one additional component selected from the group consisting of an additional active ingredient, an agriculturally acceptable adjuvant, and an agriculturally acceptable excipient.

Subject to 37 CFR § 1.808(b), all restrictions imposed by the depositor on the availability to the public of the deposited material will be irrevocably removed upon the granting of any patent from this patent application.

### EXAMPLES

The following examples are given for purely illustrative and non-limiting purposes of the present invention.

### Example 1 Methylobacterium Inoculation Effect on Promotion of Early Rice Growth

Methylobacterium isolates were tested for their ability to enhance early growth of rice seedlings. A randomized complete block design was used, with 12 treatments in each run; 10 unique Methylobacterium isolates, a Methylobacterium positive control, LGP2018, that demonstrated consistent root growth promotion of rice seedlings during assay development and increased yield levels in corn field trials (WO2020117690). The untreated control sample (UTC) was Methylobacterium growth medium applied in the same amount as used for the Methylobacterium isolates. Each treatment level had an n of 10. All 10 blocks were grown in the same growth chamber, and on the same shelf.

### Procedure

### Media:

0.5× Murashige and Skoog MS agar plates with 0.5% sucrose

### Pre-planting:

Rice seeds were de-husked. Average 100 seed count is 2018 mg with approximately 21 g of husked rice per run.

### Planting:

Seeds were sterilized in ˜3% sodium hypochlorite+0.05% Tween 20.
Seeds were washed to remove bleach solution and placed on a sterile plate lid to begin drying.
Seeds were plated using a randomized complete block design with each complete block having similarly sized seeds.
Using sterile techniques 8 sterile seeds were evenly spaced in a horizontal line (˜40% above the bottom of the plate, using a pre-marked lid as a guide). Seeds were placed with the embryo toward the bottom of the plate and gently pushed into media.

### Inoculation:

Each Methylobacterium isolate or the culture medium control was applied as an 80 uL streak to the bottom portion of the plate (one isolate per plate) and spread by gently tilting the plate back and forth. A target concentration of 1×10⁶ CFU per seed was applied.
Plates were allowed to dry for at least on hour and placed in a randomized layout in a Percival growth chamber set to 25° C. and 16 hour days.
Seeds were allowed to grow undisturbed for 8 days.

### Harvest:

At 8 days after plating the plates were removed from the growth chambers, and the plants (approximately V2 stage) were measured as follows.
Plants that were not impeded from growing normally (by physical surroundings unrelated to presence of Methylobacterium) were removed from plates, and the number of seedlings for that plate recorded.
Seedlings were scanned using WinRhizo and the images analyzed to determine root length for each plant.

The results of this experiment are shown below in Table 2.

TABLE 2

| | | | Absolute | |
|---|---|---|---|---|
| Experiment | | | Root Length | Normalized |
| Number | Treatment ID | Treatment | (cm) | Root Length |
| 264PB | 264PB LGP2018 | LGP2018 | 18.82978 | 100 |
| 264PB | 264PB Strain 1 | LGP2025 | 17.39133 | 73.325898 |
| 264PB | 264PB Strain 2 | LGP2073 | 17.19 | 69.59247 |
| 264PB | 264PB Strain 3 | LGP2047 | 16.37316 | 54.44538 |
| 264PB | 264PB Strain 4 | LGP2045 | 15.96066 | 46.796074 |
| 264PB | 264PB Strain 5 | LGP2151 | 15.39851 | 36.371618 |
| 264PB | 264PB Strain 6 | LGP2103 | 15.04489 | 29.814374 |
| 264PB | 264PB Strain 7 | LGP2125 | 14.84019 | 26.018352 |
| 264PB | 264PB Strain 8 | LGP2017 | 14.54892 | 20.61718 |
| 264PB | 264PB Strain 9 | LGP2120 | 13.84252 | 7.517937 |
| 264PB | 264PB Strain 10 | LGP2124 | 13.18279 | −4.715877 |
| 265PB | 265PB Strain 1 | LGP2071 | 14.117796 | 100.010863 |
| 265PB | 265PB LGP2018 | LGP2018 | 14.117132 | 100 |
| 265PB | 265PB Strain 2 | LGP2061 | 12.535499 | 74.124179 |
| 265PB | 265PB Strain 3 | LGP2107 | 11.83976 | 62.741755 |
| 265PB | 265PB Strain 4 | LGP2065 | 9.992807 | 32.52525 |
| 265PB | 265PB Strain 5 | LGP2051 | 9.743358 | 28.444232 |
| 265PB | 265PB Strain 6 | LGP2054 | 8.960485 | 15.636268 |
| 265PB | 265PB Strain 7 | LGP2092 | 8.856461 | 13.934427 |
| 265PB | 265PB Strain 8 | LGP2079 | 8.610079 | 9.903568 |
| 265PB | 265PB Strain 9 | LGP2052 | 7.916505 | −1.443435 |
| 266PB | 266PB Strain 1 | LGP2059 | 15.569966 | 123.451522 |
| 266PB | 266PB Strain 2 | LGP2016 | 14.587924 | 108.443799 |
| 266PB | 266PB LGP2018 | LGP2018 | 14.035398 | 100 |
| 266PB | 266PB Strain 3 | LGP2158 | 13.207394 | 87.346316 |
| 266PB | 266PB Strain 4 | LGP2066 | 12.900975 | 82.663567 |
| 266PB | 266PB Strain 5 | LGP2141 | 11.897894 | 67.334339 |
| 266PB | 266PB Strain 6 | LGP2078 | 10.298694 | 42.8951 |
| 266PB | 266PB Strain 7 | LGP2050 | 10.041706 | 38.967777 |
| 266PB | 266PB Strain 8 | LGP2080 | 9.462625 | 30.118161 |
| 266PB | 266PB Strain 9 | LGP2048 | 9.284123 | 27.390276 |
| 266PB | 266PB Strain 10 | LGP2053 | 7.207347 | −4.347354 |
| 267PB | 267PB Strain 1 | LGP2046 | 14.419073 | 137.78678 |
| 267PB | 267PB LGP2018 | LGP2018 | 12.303465 | 100 |
| 267PB | 267PB Strain 2 | LGP2024 | 11.846345 | 91.835407 |
| 267PB | 267PB Strain 3 | LGP2148 | 10.620679 | 69.94383 |
| 267PB | 267PB Strain 4 | LGP2144 | 9.415631 | 48.420528 |
| 267PB | 267PB Strain 5 | LGP2150 | 9.382432 | 47.827557 |
| 267PB | 267PB Strain 6 | LGP2110 | 9.298016 | 46.319801 |
| 267PB | 267PB Strain 7 | LGP2176 | 8.103827 | 24.990443 |
| 267PB | 267PB Strain 8 | LGP2153 | 7.128328 | 7.567103 |
| 267PB | 267PB Strain 9 | LGP2082 | 6.373293 | −5.91855 |
| 268PB | 268PB Strain 1 | LGP2021 | 15.569966 | 123.451522 |
| 268PB | 268PB Strain 2 | LGP2040 | 14.587924 | 108.443799 |
| 268PB | 268PB LGP2018 | LGP2018 | 14.035398 | 100 |
| 268PB | 268PB Strain 3 | LGP2138 | 13.207394 | 87.346316 |
| 268PB | 268PB Strain 4 | LGP2095 | 12.900975 | 82.663567 |
| 268PB | 268PB Strain 5 | LGP2130 | 11.897894 | 67.334339 |
| 268PB | 268PB Strain 6 | LGP2099 | 10.298694 | 42.8951 |
| 268PB | 268PB Strain 7 | LGP2077 | 10.041706 | 38.967777 |
| 268PB | 268PB Strain 8 | LGP2102 | 9.462625 | 30.118161 |
| 268PB | 268PB Strain 9 | LGP2072 | 9.284123 | 27.390276 |
| 268PB | 268PB Strain 10 | LGP2081 | 7.207347 | −4.347354 |
| 269PB | 269PB LGP2018 | LGP2018 | 16.079324 | 100 |
| 269PB | 269PB Strain 1 | LGP2094 | 15.70514 | 95.501874 |
| 269PB | 269PB Strain 2 | LGP2101 | 15.386634 | 91.673054 |
| 269PB | 269PB Strain 3 | LGP2090 | 14.624067 | 82.506105 |
| 269PB | 269PB Strain 4 | LGP2093 | 12.998755 | 62.967937 |
| 269PB | 269PB Strain 5 | LGP2084 | 12.830224 | 60.942001 |
| 269PB | 269PB Strain 6 | LGP2114 | 12.516872 | 57.175138 |
| 269PB | 269PB Strain 7 | LGP2100 | 11.343389 | 43.068489 |
| 269PB | 269PB Strain 8 | LGP2085 | 9.828333 | 24.855728 |
| 269PB | 269PB Strain 9 | LGP2075 | 7.587342 | −2.08362 |
| 269PB | 269PB Strain 10 | LGP2083 | 7.50976 | −3.016248 |
| 270PB | 270PB Strain 1 | LGP2029 | 14.570904 | 104.017951 |
| 270PB | 270PB LGP2018 | LGP2018 | 14.31934 | 100 |
| 270PB | 270PB Strain 2 | LGP2135 | 13.363759 | 84.737607 |
| 270PB | 270PB Strain 3 | LGP2129 | 12.594344 | 72.448632 |
| 270PB | 270PB Strain 4 | LGP2143 | 10.608781 | 40.735534 |
| 270PB | 270PB Strain 5 | LGP2137 | 10.04973 | 31.806444 |
| 270PB | 270PB Strain 6 | LGP2128 | 9.970479 | 30.540667 |
| 270PB | 270PB Strain 7 | LGP2123 | 9.933589 | 29.951459 |
| 270PB | 270PB Strain 8 | LGP2126 | 9.635704 | 25.193695 |
| 270PB | 270PB Strain 9 | LGP2136 | 9.506136 | 23.124249 |
| 270PB | 270PB Strain 10 | LGP2121 | 7.872883 | −2.961817 |
| 271PB | 271PB LGP2018 | LGP2018 | 18.545695 | 100 |
| 271PB | 271PB Strain 1 | LGP2069 | 16.856945 | 83.10707 |
| 271PB | 271PB Strain 2 | LGP2027 | 15.948911 | 74.02381 |
| 271PB | 271PB Strain 3 | LGP2056 | 14.750148 | 62.03233 |
| 271PB | 271PB Strain 4 | LGP2096 | 14.330543 | 57.83493 |
| 271PB | 271PB Strain 5 | LGP2060 | 13.874818 | 53.27622 |
| 271PB | 271PB Strain 6 | LGP2097 | 13.443795 | 48.9646 |
| 271PB | 271PB Strain 7 | LGP2067 | 13.24211 | 46.9471 |
| 271PB | 271PB Strain 8 | LGP2055 | 12.770669 | 42.23118 |
| 271PB | 271PB Strain 9 | LGP2086 | 12.549608 | 40.01986 |
| 271PB | 271PB Strain 10 | LGP2057 | 11.572393 | 30.24456 |
| 273PB | 273PB LGP2018 | LGP2018 | 13.216513 | 100 |
| 273PB | 273PB Strain 1 | LGP2028 | 11.289892 | 71.38989 |
| 273PB | 273PB Strain 2 | LGP2098 | 10.957287 | 66.45074 |
| 273PB | 273PB Strain 3 | LGP2116 | 10.552009 | 60.43241 |
| 273PB | 273PB Strain 4 | LGP2131 | 10.492209 | 59.54438 |
| 273PB | 273PB Strain 5 | LGP2117 | 9.92343 | 51.09808 |
| 273PB | 273PB Strain 6 | LGP2133 | 9.207299 | 40.46361 |
| 273PB | 273PB Strain 7 | LGP2140 | 9.188468 | 40.18397 |
| 273PB | 273PB Strain 8 | LGP2134 | 8.651127 | 32.20451 |
| 273PB | 273PB Strain 9 | LGP2109 | 7.244746 | 11.31992 |
| 273PB | 273PB Strain 10 | LGP2111 | 5.404409 | −16.0089 |
| 274PB | 274PB Strain 1 | LGP2033 | 17.459903 | 136.108331 |
| 274PB | 274PB Strain 2 | LGP2118 | 15.623786 | 106.167536 |
| 274PB | 274PB LGP2018 | LGP2018 | 15.245562 | 100 |
| 274PB | 274PB Strain 3 | LGP2145 | 14.631981 | 89.994584 |
| 274PB | 274PB Strain 4 | LGP2032 | 14.299443 | 84.572029 |
| 274PB | 274PB Strain 5 | LGP2152 | 13.881329 | 77.754029 |
| 274PB | 274PB Strain 6 | LGP2147 | 13.409769 | 70.064484 |
| 274PB | 274PB Strain 7 | LGP2157 | 11.306689 | 35.770445 |
| 274PB | 274PB Strain 8 | LGP2142 | 10.1196 | 16.413079 |
| 274PB | 274PB Strain 9 | LGP2159 | 9.361136 | 4.045128 |
| 274PB | 274PB Strain 10 | LGP2154 | 8.943802 | −2.760155 |
| 275PB | 275PB LGP2018 | LGP2018 | 18.826053 | 100 |
| 275PB | 275PB Strain 1 | LGP2022 | 17.00802 | 80.576456 |
| 275PB | 275PB Strain 2 | LGP2023 | 16.310993 | 73.129541 |
| 275PB | 275PB Strain 3 | LGP2160 | 15.87016 | 68.41976 |
| 275PB | 275PB Strain 4 | LGP2163 | 15.337422 | 62.728087 |
| 275PB | 275PB Strain 5 | LGP2167 | 15.162438 | 60.858589 |
| 275PB | 275PB Strain 6 | LGP2166 | 14.298438 | 51.627764 |
| 275PB | 275PB Strain 7 | LGP2161 | 13.02194 | 37.989883 |
| 275PB | 275PB Strain 8 | LGP2162 | 11.85523 | 25.52496 |
| 275PB | 275PB Strain 9 | LGP2168 | 10.190812 | 7.742619 |
| 277PB | 277PB LGP2018 | LGP2018 | 15.854562 | 100 |
| 277PB | 277PB Strain 1 | LGP2062 | 14.420103 | 81.45296 |
| 277PB | 277PB Strain 2 | LGP2185 | 14.124727 | 77.63385 |
| 277PB | 277PB Strain 3 | LGP2063 | 13.598758 | 70.83327 |
| 277PB | 277PB Strain 4 | LGP2074 | 12.56993 | 57.53088 |
| 277PB | 277PB Strain 5 | LGP2058 | 12.237293 | 53.23002 |
| 277PB | 277PB Strain 6 | LGP2064 | 11.790611 | 47.45458 |
| 277PB | 277PB Strain 7 | LGP2091 | 11.598483 | 44.97043 |
| 277PB | 277PB Strain 8 | LGP2186 | 10.193847 | 26.809 |
| 277PB | 277PB Strain 9 | LGP2105 | 10.166668 | 26.45758 |
| 277PB | 277PB Strain 10 | LGP2187 | 10.018778 | 24.54541 |
| 282PB | 282PB LGP2018 | LGP2018 | 17.115992 | 100 |
| 282PB | 282PB Strain 1 | LGP2087 | 15.150588 | 77.27183 |
| 282PB | 282PB Strain 2 | LGP2108 | 14.929319 | 74.71305 |
| 282PB | 282PB Strain 3 | LGP2076 | 14.913514 | 74.53028 |
| 282PB | 282PB Strain 4 | LGP2106 | 13.131888 | 53.92734 |
| 282PB | 282PB Strain 5 | LGP2113 | 12.547632 | 47.17093 |
| 282PB | 282PB Strain 6 | LGP2049 | 12.529399 | 46.96009 |
| 282PB | 282PB Strain 7 | LGP2068 | 12.507406 | 46.70576 |
| 282PB | 282PB Strain 8 | LGP2149 | 12.28271 | 44.10735 |
| 282PB | 282PB Strain 9 | LGP2005 | 11.888991 | 39.55433 |
| 282PB | 282PB Strain 10 | LGP2006 | 10.285192 | 21.00781 |
| 283PB | 283PB Strain 1 | LGP2182 | 14.59702 | 103.904114 |
| 283PB | 283PB LGP2018 | LGP2018 | 14.364828 | 100 |
| 283PB | 283PB Strain 2 | LGP2034 | 13.842152 | 91.211673 |
| 283PB | 283PB Strain 3 | LGP2146 | 12.351052 | 66.14017 |
| 283PB | 283PB Strain 4 | LGP2181 | 12.117376 | 62.211111 |
| 283PB | 283PB Strain 5 | LGP2089 | 11.13865 | 45.754717 |
| 283PB | 283PB Strain 6 | LGP2156 | 10.858914 | 41.051207 |
| 283PB | 283PB Strain 7 | LGP2170 | 10.110786 | 28.472101 |
| 283PB | 283PB Strain 8 | LGP2155 | 9.582397 | 19.587708 |
| 283PB | 283PB Strain 9 | LGP2127 | 8.857205 | 7.394253 |
| 283PB | 283PB Strain 10 | LGP2139 | 8.755959 | 5.691884 |
| 285PB | 285PB LGP2018 | LGP2018 | 12.031742 | 100 |
| 285PB | 285PB Strain 1 | LGP2173 | 11.21333 | 84.0138457 |
| 285PB | 285PB Strain 2 | LGP2172 | 10.228408 | 64.7752232 |
| 285PB | 285PB Strain 3 | LGP2164 | 9.964949 | 59.6290516 |
| 285PB | 285PB Strain 4 | LGP2165 | 9.033842 | 41.4416163 |
| 285PB | 285PB Strain 5 | LGP2008 | 7.982016 | 20.8961413 |
| 285PB | 285PB Strain 6 | LGP2112 | 7.609441 | 13.6186008 |
| 285PB | 285PB Strain 7 | LGP2169 | 7.485808 | 11.2036581 |
| 285PB | 285PB Strain 8 | LGP2044 | 7.402148 | 9.5695127 |
| 285PB | 285PB Strain 9 | LGP2011 | 6.922695 | 0.2042973 |
| 285PB | 285PB Strain 10 | LGP2171 | 5.864521 | −20.4651746 |
| 286PB | 286PB Strain 1 | LGP2001 | 18.47052 | 102.4019 |
| 286PB | 286PB LGP2018 | LGP2018 | 18.29094 | 100 |
| 286PB | 286PB Strain 2 | LGP2012 | 17.23022 | 85.81258 |
| 286PB | 286PB Strain 3 | LGP2000 | 17.06282 | 83.57344 |
| 286PB | 286PB Strain 4 | LGP2015 | 16.97065 | 82.34073 |
| 286PB | 286PB Strain 5 | LGP2007 | 15.82329 | 66.99432 |
| 286PB | 286PB Strain 6 | LGP2003 | 14.07074 | 43.5534 |
| 286PB | 286PB Strain 7 | LGP2010 | 14.04739 | 43.24119 |
| 286PB | 286PB Strain 8 | LGP2013 | 13.72635 | 38.9471 |
| 286PB | 286PB Strain 9 | LGP2004 | 12.51197 | 22.7044 |
| 288PB | 288PB Strain 1 | LGP2031 | 11.73032 | 115.04974 |
| 288PB | 288PB LGP2018 | LGP2018 | 10.961572 | 100 |
| 288PB | 288PB Strain 2 | LGP2030 | 10.823393 | 97.29486 |
| 288PB | 288PB Strain 3 | LGP2184 | 10.428576 | 89.56555 |
| 288PB | 288PB Strain 4 | LGP2188 | 10.060309 | 82.35601 |
| 288PB | 288PB Strain 5 | LGP2132 | 10.004185 | 81.25727 |
| 288PB | 288PB Strain 6 | LGP2179 | 9.603427 | 73.41165 |
| 288PB | 288PB Strain 7 | LGP2183 | 9.371095 | 68.86329 |
| 288PB | 288PB Strain 8 | LGP2122 | 8.820766 | 58.08953 |
| 288PB | 288PB Strain 9 | LGP2009 | 7.664263 | 35.44871 |
| 288PB | 288PB Strain 10 | LGP2088 | 6.600541 | 14.62428 |
| 289PB | 289PB Strain 1 | LGP2002 | 16.64733 | 117.25169 |
| 289PB | 289PB LGP2018 | LGP2018 | 15.73919 | 100 |
| 289PB | 289PB Strain 2 | LGP2174 | 14.52193 | 76.87615 |
| 289PB | 289PB Strain 3 | LGP2178 | 14.47025 | 75.89433 |
| 289PB | 289PB Strain 4 | LGP2119 | 14.41787 | 74.89923 |
| 289PB | 289PB Strain 5 | LGP2070 | 14.39551 | 74.47451 |
| 289PB | 289PB Strain 6 | LGP2104 | 14.2175 | 71.09291 |
| 289PB | 289PB Strain 7 | LGP2175 | 13.17078 | 51.20856 |
| 289PB | 289PB Strain 8 | LGP2115 | 13.15135 | 50.83953 |
| 289PB | 289PB Strain 9 | LGP2177 | 13.0369 | 48.66526 |
| 289PB | 289PB Strain 10 | LGP2180 | 13.00762 | 48.10911 |

Forty-eight Methylobacterium strains were selected for gene correlation analysis from the 176 strains tested, including 15 non-hits and 33 hits. The strains were selected from those having the highest and lowest normalized root scores, excluding any isolates that had any signs of any type of microbial contamination. The normalized score standardized each isolate's mean root length value to the UTC (a value of 0) and the positive control, LGP2018 (a value of 100).

Genomes of the selected isolates were assembled and putative genes identified. The genes were assigned a putative function by sequence analysis to databases of known genes and gene signatures. A pan-genome for Methylobacterium was constructed as described by Page et al. (Roary: rapid large-scale prokaryote pan genome analysis, Bioinformatics (2015) 31:3691-3693) except that genome sequences from greater than 1000 different species of Methylobacterium were assembled and used to construct the pan-genome as opposed to the single Salmonella species described by Page et al.

The genomes of strains identified as enhancing rice seedling growth, “hits”, and strains identified as “non-hits”, were compared to determine the presence or absence in each strain of each genetic element in the pan-genome. For this analysis, translated genes were clustered across strains using BLASTP with a sequence identity of at least 50% to identify homologous genetic elements across genomes. These results were used to determine which genetic elements are the same or different across strains, leading to a score for each genetic element as present or absent in a given strain. The presence/absence scores were used in a correlation analysis to identify genetic elements that correlate positively with enhancing rice seedling growth as described by Brynildsrud et al. (Rapid scoring of genes in microbial pan-genome-wide association studies with Scoary, Genome Biology (2016) 17:238).

The steps in the process were as follows. Correlated genetic elements were collapsed so that genes that are typically inherited together, for example genes on the same plasmid, were combined into a single unit. Each genetic element in the pan-genome received a null hypothesis of no association to the trait. A Fisher's exact test was performed on each genetic element with the assumption that all strains had a random and independently distributed probability for exhibiting each state, i.e. presence or absence of the genetic element. To control spurious associations due to population structure, the pairwise comparisons algorithm was applied using a phylogenetic tree of the Methylobacterium genus, constructed using the same genome sequences described above. Empirical p-value was computed using label-switching permutations, i.e. the test statistic was generated over random permutations of the phenotype data. The genetic elements that were significantly positively correlated with enhancing rice seedling root growth were identified based on p value using a threshold for statistical significance of p less than or equal to 0.05. Sensitivity and specificity cutoffs were also employed based on the number of hits and non-hits a gene was present in.

Gene elements that were positively correlated with Methylobacterium enhancement of growth in rice seedlings are shown in Table 3A below.

TABLE 3A

| | Consensus | | | | | |
|---|---|---|---|---|---|---|
| | Protein | Representative | | | | |
| Gene | SEQ ID | protein | | | | p- |
| name | NO: | sequences | Annotation | Sensitivity | Specificity | value |
| group_4403 | 17 | SEQ 24 | hypothetical protein | 60.61 | 80.00 | 0.003 |
| group_9931 | 18 | SEQ 25 | hypothetical protein | 57.58 | 86.67 | 0.025 |
| group_7199 | 19 | SEQ 26 | hypothetical protein | 66.67 | 86.67 | 0.030 |
| recD2_2 | 20 | SEQ 27 | ATP-dependent RecD- | 45.45 | 93.33 | 0.035 |
| | | | like DNA helicase | | | |
| pinR | 21 | SEQ 28 | Putative DNA- | 69.70 | 80.00 | 0.039 |
| | | | invertase from | | | |
| | | | lambdoid prophage Rac | | | |
| group_2780 | 22 | SEQ 29 | hypothetical protein | 33.33 | 100.00 | 0.055 |
| group_5546 | 23 | SEQ 30 | hypothetical protein | 60.61 | 80.00 | 0.057 |

Methylobacterium consensus protein sequences for the above identified genes that positively correlate with enhanced growth or rice seedlings are provided as SEQ ID NO: 17 through SEQ ID NO:23 disclosed in the Sequence Listing. Consensus sequences are generated by aligning the encoded protein sequences from all isolates from a comprehensive database of Methylobacterium genome sequences from public and internal databases. EMBOSS cons was used to generate consensus sequences from the multiple sequence alignment. Where no consensus was found at a position an ‘x’ character is used. An upper case letter for an amino acid residue indicates that most of the sequences have that amino acid at that position. In the consensus sequences, X can be any amino acid residue or can be absent.

Representative amino acid sequences for proteins correlated with enhancing growth of rice seedlings from specific Methylobacterium strains are provided below as SEQ ID NOs: 24-30 and SEQ ID NOs: 123-128. The strain from which a representative sequence was obtained is referenced below in Table 3B.

TABLE 3B

| Strain | Amino Acid SEQ ID NO |
|---|---|
| LGP2022 | 24 |
| LGP2021 | 25 |
| LGP2021 | 26 |
| LGP2021 | 27 |
| LGP2022 | 28 |
| LGP2016 | 29 |
| LGP2022 | 30 |
| LGP2004 | 123 |
| LGP2020 | 124 |
| LGP2020 | 125 |
| LGP2020 | 126 |
| LGP2020 | 127 |
| LGP2017 | 128 |

### Example 2 Methylobacterium Inoculation Effect on Nitrogen Utilization in Rice

Methylobacterium isolates were tested for their ability to enhance shoot nitrogen content and/or concentration in rice. A randomized complete block design was used, with 12 treatments in each run; five Methylobacterium isolates and a control at two nitrogen levels. The untreated control sample (UTC) was Methylobacterium growth medium applied in the same amount as used for the Methylobacterium isolates. Each treatment level had an n of 10. All 10 blocks were grown in the same growth chamber and on the same shelf.

### Procedure

### Media:

0.5× Murashige and Skoog MS medium with high or low nitrogen
   High nitrogen media—10400 uM
   Low nitrogen media—250 uM

### Pre-planting:

Rice seeds were de-husked. Average 100 seed count is 2018 mg with approximately 21 g of husked rice per run.
Agar plates containing high or low nitrogen media were prepared.

### Planting:

Seeds were sterilized in ˜3% sodium hypochlorite+0.05% Tween 20.
Seeds were washed to remove bleach solution and placed on a sterile plate lid to begin drying.
Seeds were plated using a randomized complete block design with each complete block having similarly sized seeds.
Using sterile techniques 8 sterile seeds were evenly spaced in a horizontal line (˜40% above the bottom of the plate, using a pre-marked lid as a guide). Seeds were placed with the embryo toward the bottom of the plate and gently pushed into media.

### Inoculation:

Each Methylobacterium isolate or the culture medium control was applied as an 80 uL streak to the bottom portion of the plate (one isolate per plate) and spread by gently tilting the plate back and forth. A target concentration of 1×10⁶ CFU per seed was applied.
Plates were allowed to dry for at least on hour and placed in a randomized layout in a Percival growth chamber set to 25° C. and 16 hour days.
Seeds were allowed to grow undisturbed for 8 days.

### Harvest:

At 8 days after plating the plates were removed from the growth chambers, and the plants were measured as follows.
Plants that were not impeded from growing normally (by physical surroundings unrelated to presence of Methylobacterium) were removed from plates, and the number of seedlings for that plate recorded.
Seedlings were scanned using WinRhizo and the images analyzed to determine root and shoot area for each plant.
Seedlings were rinsed to remove any remaining plate media and the shoots separated from the seedlings and dried in a drying oven for at least 3 days.
Dried shoots were combined for each treatment and the mass measured. The plant material was then ground to a powder to be used for nitrogen testing.
Nitrogen analysis was conducted on the powdered samples by Atlantic Microlab (Norcross, GA).

Results of the analyses are shown below. In all tables, pairwise results are presented separately for the High N and Low N treatments. Data was analyzed using Student's t-test and different letters indicate a significant difference between treatments at p<0.05.

TABLE 4
Exp 1 Shoot Area Measurements

| | |
|---|---|
| | Mean Shoot Area |
| Treatment | per Plant (cm²) |

4A Low Nitrogen Treatment

| | | |
|---|---|---|
| LGP2033 | A | 0.30 |
| UTC | A | 0.30 |
| LGP2009 | A | 0.29 |
| LGP2020 | A | 0.29 |
| LGP2022 | A | 0.28 |
| LGP2003 | A | 0.28 |

4B High Nitrogen Treatment

| | | |
|---|---|---|
| LGP2020 | A | 0.51 |
| LGP2033 | B | 0.42 |
| LGP2022 | BC | 0.40 |
| LGP2003 | BC | 0.40 |
| UTC | BC | 0.36 |
| LGP2009 | C | 0.34 |

TABLE 5
Exp 1 Root Area Measurements

| | |
|---|---|
| | Mean Root Area |
| Treatment | per Plant (cm²) |

5A Low Nitrogen Treatment

| | | |
|---|---|---|
| LGP2020 | A | 0.93 |
| LGP2022 | A | 0.88 |
| LGP2033 | AB | 0.85 |
| LGP2009 | B | 0.79 |
| LGP2003 | B | 0.77 |
| UTC | C | 0.64 |

5B High Nitrogen Treatment

| | | |
|---|---|---|
| LGP2020 | A | 0.99 |
| LGP2022 | B | 0.85 |
| LGP2033 | B | 0.83 |
| LGP2003 | C | 0.67 |
| LGP2009 | C | 0.62 |
| UTC | C | 0.59 |

TABLE 6
Exp 1 Shoot Nitrogen Concentration

| | |
|---|---|
| | Mean % Dry Wt |
| Treatment | Nitrogen |

6A Low Nitrogen Treatment

| | | |
|---|---|---|
| UTC | A | 2.73 |
| LGP2020 | B | 2.59 |
| LGP2022 | C | 2.48 |
| LGP2033 | C | 2.49 |
| LGP2009 | D | 2.35 |
| LGP2003 | D | 2.30 |

6B High Nitrogen Treatment

| | | |
|---|---|---|
| LGP2020 | A | 4.92 |
| LGP2022 | B | 4.38 |
| LGP2033 | C | 4.02 |
| UTC | D | 3.23 |
| LGP2009 | D | 3.27 |
| LGP2003 | D | 3.26 |

Significant and substantial shoot growth promotion was observed for some isolates at high nitrogen. Shoot growth promotion was not observed for the

Methylobacterium treatments at low nitrogen, consistent with some literature reports which indicate that growth promotion effects from plant-beneficial microbes may not be observed when nutrient availability is too low. Root growth promotion was evident at both nitrogen levels and Root/Shoot ratios are higher under low N than under high N. As expected, plants grown on high N media showed substantially greater shoot N concentration than those grown on low N media. Several Methylobacterium isolates demonstrated significantly enhanced shoot nitrogen concentration under high nitrogen growth conditions. Three isolates, LGP2020, LGP2022, and LGP2033, demonstrated the greatest enhancements of shoot growth, root growth and shoot nitrogen concentration.

The above experiment was repeated using four of the same Methylobacterium isolates and one additional isolate. Results were similar to those observed in the first assay and are shown in the tables below. LGP2020 (NRRL-B-67892), LGP2022 (NRRL-B-68033), and LGP2033, again demonstrated enhancements of shoot growth, root growth and shoot nitrogen concentration.

TABLE 7
Exp 2 Shoot Area Measurements

| | |
|---|---|
| | Mean Shoot Area |
| Treatment | per Plant (cm²) |

7A Low Nitrogen Treatment

| | | |
|---|---|---|
| LGP2022 | A | 0.18 |
| LGP2033 | A | 0.19 |
| LGP2020 | A | 0.17 |
| UTC | A | 0.19 |
| LGP2003 | A | 0.18 |
| LGP2019 | A | 0.18 |

7B High Nitrogen Treatment

| | | |
|---|---|---|
| LGP2022 | A | 0.30 |
| LGP2033 | AB | 0.30 |
| LGP2020 | AB | 0.29 |
| UTC | AB | 0.26 |
| LGP2003 | AB | 0.25 |
| LGP2019 | B | 0.25 |

TABLE 8
Exp 2 Root Area Measurements

| | |
|---|---|
| | Mean Root Area |
| Treatment | per Plant (cm²) |

8A Low Nitrogen Treatment

| | | |
|---|---|---|
| LGP2033 | AB | 0.57 |
| LGP2022 | AB | 0.53 |
| LGP2020 | A | 0.59 |
| LGP2019 | AB | 0.56 |
| LGP2003 | AB | 0.52 |
| UTC | B | 0.50 |

8B High Nitrogen Treatment

| | | |
|---|---|---|
| LGP2033 | A | 0.67 |
| LGP2022 | A | 0.66 |
| LGP2020 | A | 0.64 |
| LGP2019 | B | 0.54 |
| LGP2003 | B | 0.49 |
| UTC | B | 0.47 |

TABLE 9
Exp 2 Shoot Nitrogen Concentration

| | |
|---|---|
| | Mean % Dry Wt |
| Treatment | Nitrogen |

9A Low Nitrogen Treatment

| | | |
|---|---|---|
| LGP2020 | AB | 2.36 |
| LGP2022 | AB | 2.30 |
| LGP2033 | AB | 2.38 |
| UTC | A | 2.51 |
| LGP2003 | B | 2.25 |
| LGP2019 | B | 2.21 |

9B High Nitrogen Treatment

| | | |
|---|---|---|
| LGP2020 | A | 4.28 |
| LGP2022 | A | 4.06 |
| LGP2033 | B | 3.68 |
| UTC | BC | 3.45 |
| LGP2003 | C | 3.37 |
| LGP2019 | C | 3.23 |

Percent difference between Methylobacterium treatments and UTC at high and low N for 3 different variables: projected root area, projected shoot area and foliar nitrogen concentration, are shown for each experiment. Bold italics are used to denote a statistically significant difference from UTC at p<0.05 using Student's t-test.

TABLE 10
Percent Differences

| | | | | | | % N | % N |
|---|---|---|---|---|---|---|---|
| N | | % Root | % Root | % Shoot GP | % Shoot GP | Enhancement | Enhancement |
| Level | Treatment | GP Exp 1 | GP Exp 2 | Exp 1 | Exp 2 | Exp 1 | Exp 2 |
| High | LGP2003 | +15.1% | +2.8% | +10.6% | −1.7% | −0.8% | −2.2% |
| N | LGP2020 | +68.5% | +35.0% | +42.0% | +14.0% | +49.7% | +23.9% |
| | LGP2033 | +41.6% | +42.2% | +16.2% | +15.5% | +22.4% | +6.8% |
| | LGP2022 | +45.4% | +40.1% | +10.8% | +15.8% | +33.3% | +17.7% |
| Low | LGP2003 | +19.4% | +4.5% | −8.9% | −8.6% | −15.8% | −10.2% |
| N | LGP2020 | +43.5% | +18.3% | −3.2% | −11.5% | −5.3% | −6.1% |
| | LGP2033 | +31.8% | +13.8% | +0.7% | −2.5% | −9.1% | −5.0% |
| | LGP2022 | +37.0% | +6.1% | −8.6% | −8.5% | −9.0% | −8.3% |

### Example 3 Evaluation of Optimal Nitrogen Dose for Testing Methylobacterium Effect

The high nitrogen dose in the experiments described above is the amount in 0.5×MS media, a general plant growth medium, and provides the optimal amount of nitrogen for plant growth. To evaluate plant response to Methylobacterium treatment under various reduced nitrogen levels, including a nitrogen level that approximates the amount of nitrogen in a field treated with a 25-30% reduction of optimal nitrogen level, two low nitrogen dose experiments were conducted.

Nitrogen doses used for evaluation of effect of Methylobacterium treatment on plant growth were: 5200 uM nitrogen (50% of rice optimal nitrogen level), 7280 uM nitrogen (70% of rice optimal nitrogen level) and 10400 uM nitrogen (100% of rice optimal nitrogen level). Results are shown in Tables 11-13 below. Data was analyzed using Student's t-test and different letters indicate a significant difference between treatments at p<0.05.

TABLE 11
Exp 3 Shoot Area Measurements

| | 5200 μM N | | 7280 μM N | | 10400 μM N | |
|---|---|---|---|---|---|---|
| | Treatment Mean | | Treatment Mean | | Treatment Mean Shoot | |
| | Shoot Area per Plant | | Shoot Area per Plant | | Area per Plant | |
| Treatment | (cm²) | | (cm²) | | (cm²) | |
| LGP2020 | A | 0.41 | A | 0.36 | A | 0.41 |
| LGP2033 | B | 0.33 | A | 0.34 | B | 0.34 |
| Control | C | 0.28 | B | 0.25 | BC | 0.30 |
| LGP2019 | C | 0.27 | B | 0.28 | C | 0.28 |

TABLE 12
Exp 3 Root Area Measurements

| | 5200 μM N | | 7280 μM N | | 10400 μM N | |
|---|---|---|---|---|---|---|
| | Treatment Mean | | Treatment Mean | | Treatment Mean Root | |
| | Root Area per Plant | | Root Area per Plant | | Area per Plant | |
| Treatment | (cm²) | | (cm²) | | (cm²) | |
| LGP2020 | A | 0.82 | A | 0.78 | A | 0.79 |
| LGP2033 | B | 0.70 | A | 0.77 | B | 0.71 |
| LGP2019 | B | 0.62 | B | 0.64 | C | 0.57 |
| Control | C | 0.47 | C | 0.45 | D | 0.49 |

TABLE 13
Exp 3 Shoot Nitrogen Concentration

| | 5200 μM N | | 7280 μM N | | 10400 μM N | |
|---|---|---|---|---|---|---|
| | Treatment Mean % | | Treatment Mean % | | Treatment Mean % | |
| Treatment | Dry Wt Nitrogen | | Dry Wt Nitrogen | | Dry Wt Nitrogen | |
| LGP2020 | A | 4.70 | A | 4.40 | A | 4.61 |
| LGP2033 | B | 3.77 | B | 4.02 | B | 3.96 |
| LGP2019 | C | 3.14 | C | 3.42 | C | 3.41 |
| Control | C | 3.13 | C | 3.22 | C | 3.34 |

Nitrogen doses used for evaluation of effect of Methylobacterium treatment on plant growth were: 1560 uM nitrogen (15% of rice optimal nitrogen level), 2600 uM nitrogen (25% of rice optimal nitrogen level) and 5200 uM nitrogen. (50% of rice optimal nitrogen level). Results are shown in Tables 14-16 below.

TABLE 14
Exp 4 Shoot Area Measurements

| | 1560 μM N | | 2600 μM N | | 5200 μM N Treatment | |
|---|---|---|---|---|---|---|
| | Treatment Mean | | Treatment Mean | | Mean Shoot Area per | |
| | Shoot Area per Plant | | Shoot Area per Plant | | Plant | |
| Treatment | (cm²) | | (cm²) | | (cm²) | |
| LGP2020 | A | 0.28 | A | 0.32 | A | 0.38 |
| LGP2017 | A | 0.27 | AB | 0.28 | AB | 0.31 |
| LGP2019 | AB | 0.26 | B | 0.26 | B | 0.26 |
| Control | B | 0.23 | C | 0.22 | B | 0.25 |

TABLE 15
Exp 4 Root Area Measurements

| | 1560 μM N | | 2600 μM N | | 5200 μM N Treatment | |
|---|---|---|---|---|---|---|
| | Treatment Mean | | Treatment Mean | | Mean Root Area per | |
| | Root Area per Plant | | Root Area per Plant | | Plant | |
| Treatment | (cm²) | | (cm²) | | (cm²) | |
| LGP2020 | A | 0.75 | A | 0.73 | A | 0.71 |
| LGP2017 | AB | 0.72 | B | 0.65 | AB | 0.66 |
| LGP2019 | B | 0.65 | B | 0.63 | B | 0.61 |
| Control | C | 0.45 | C | 0.44 | C | 0.45 |

TABLE 16
Exp 4 Shoot Nitrogen Concentration

| | 1560 μM N | | 2600 μM N | | 5200 μM N Treatment | |
|---|---|---|---|---|---|---|
| | Treatment Mean % | | Treatment Mean % | | Mean % Dry Wt | |
| Treatment | Dry Wt Nitrogen | | Dry Wt Nitrogen | | Nitrogen | |
| LGP2020 | A | 3.03 | A | 3.65 | A | 4.67 |
| LGP2017 | A | 3.00 | B | 3.51 | B | 4.22 |
| LGP2019 | AB | 2.86 | C | 3.30 | C | 3.25 |
| Control | B | 2.73 | D | 2.90 | C | 3.15 |

Results again demonstrate significant and substantial shoot and root growth promotion and increased levels of shoot nitrogen levels resulting from treatment with Methylobacterium isolates. Shoot area correlated closely to nitrogen levels measured in shoots. Although root area measurements were not observed to be in proportion to increased nitrogen uptake as measured in shoots, additional observations noted that numbers of root tips were increased in line with enhanced nitrogen uptake as measured in shoot nitrogen concentration.

Experiments to identify additional Methylobacterium strains that can enhance plant growth and development under reduced nitrogen levels will be conducted using a 7280 μM nitrogen treatment, representing 70% of the optimal N level for rice, or a 30% reduction in nitrogen fertilizer application for rice cultivation.

### Example 4. Increases in Rice Yield by Application of Methylobacterium

Rice field trials were conducted at three locations, all near Humphrey, AR, for the purpose of evaluating the effects of three Methylobacterium isolates applied as a seed treatment. Treatments included each Methylobacterium isolate and an untreated control applied to rice seeds with and without a base treatment of insecticide only (active ingredient Clothianidin). The trial was conducted using a Randomized Complete Block Design (RCBD) with 4 reps per location. LGP2016 (NRRL B-67341), LGP2019 (NRRL B-67743) and LGP2017 (NRRL B-67741) were applied to rice seeds at a target concentration of 10⁶ CFU/seed.

The Methylobacterium isolates increased yield in rice field trials as compared to the untreated control both with and without insecticide treatment as shown in the Table below.

TABLE 17
Mean yield (Bu/A) Increase over control and percent increase shown

| Treatment | UTC | LGP2016 | | LGP2019 | | LGP2017 | |
|---|---|---|---|---|---|---|---|
| Without | 143.8 | 150.1 | +6.3 (4.3%) | 156.2 | +12.4 (8.6%) | 152.4 | +8.6 (6.0%) |
| insecticide | | | | | | | |
| treatment | | | | | | | |
| With | 151.8 | 164.3 | +12.5 (8.2%) | 155.4 | +3.6 (2.4%) | 158.2 | +6.4 (4.2%) |
| insecticide | | | | | | | |
| treatment | | | | | | | |

(Bold italics indicates a significant difference at p < 0.05 using Fisher's LSD test.)

Also provided herein are methods of improving growth and yield of rice plants by treating rice plants, plant parts or seeds with one or more Methylobacterium isolates. In some embodiments, harvested seed yield and/or nutrient content of rice plants is improved. In some embodiments, rice seeds are treated and such treatment provides for increased rice seed yield. In some embodiments, the Methylobacterium isolate is selected from the group consisting of LGP2016 (NRRL B-67341), LGP2017 (NRRL B-67741), LGP2019 (NRRL B-67743) and variants of these isolates. Rice plants, plant parts or seeds coated with Methylobacterium isolates and/or compositions are also provided herein. In certain embodiments, the Methylobacterium has chromosomal genomic DNA having at least 99%, 99.9, 99.8, 99.7, 99.6%, or 99.5% sequence identity to chromosomal genomic DNA of LGP2016, LGP2017, or LGP2019. In certain embodiments, the Methylobacterium has genomic DNA comprising one or more polynucleotide marker fragments of at least 50, 60, 100, 120, 180, 200, 240, or 300 nucleotides of SEQ ID NOS: 37-39 or SEQ ID NOS: 25-27.

### Example 5. Procedure to Test Methylobacterium Inoculation Effect on Nitrogen Utilization in Rice

Additional Methylobacterium strains, including Methylobacterium strains that caused increased root length during early rice growth from Example 1, are tested for Methylobacterium inoculation effect on nitrogen utilization in rice.

The experiment is conducted replacing the high and low nitrogen conditions with using 7280 uM nitrogen (70% of rice optimal nitrogen level). Data can be analyzed using Student's t-test to determine significant differences between strains at p<0.05 to determine strains that have increased nitrogen uptake compared to untreated control samples.

Results shown in Table 18 below provide percent differences in foliar N concentration in treated rice plants compared to N levels in untreated seedlings. Foliar tissue was harvested, dried, and assayed for nitrogen concentration via elemental combustion analysis.

TABLE 18

| | Percent difference from | |
|---|---|---|
| Methylobacterium | Untreated in Foliar N | Number of |
| Strain | concentration (% by mass) | times tested |
| LGP2020 | +45.2% | 9 |
| LGP2023 | +47.6% | 1 |
| LGP2031 | +38.2% | 3 |
| LGP2034 | +43.9% | 1 |
| LGP2029 | +35.7% | 3 |
| LGP2021 | +41.0% | 1 |
| LGP2167 | +40.5% | 1 |
| LGP2030 | +32.0% | 3 |
| LGP2002 | +42.8% | 1 |
| LGP2018 | +37.5% | 1 |
| LGP2001 | +29.2% | 1 |
| LGP2015 | +27.9% | 1 |
| LGP2188 | +3.0% | 1 |
| LGP2189 | −4.8% | 1 |
| LGP2005 | −4.9% | 1 |
| LGP2004 | −4.7% | 1 |
| LGP2032 | +30.0% | 1 |
| LGP2024 | +31.3% | 1 |
| NLS0681 | +27.2% | 1 |
| NLS0594 | +24.2% | 1 |
| NLS0479 | +43.2% | 1 |
| NLS1310 | +44.2% | 1 |
| NLS0612 | +38.1% | 1 |
| NLS1312 | +36.5% | 1 |
| NLS0473 | +32.6% | 1 |
| NLS0706 | +34.5% | 1 |
| NLS0725 | +34.9% | 1 |
| NLS0159 | +5.1% | 1 |
| NLS0229 | −1.5% | 1 |

### Example 6: Growth of Methylobacterium Isolates Containing Methane Monooxygenase (pMMO) Genes Using Methane as Sole Carbon Source

Methylobacterium strains and positive and negative controls are grown on ammonium mineral salts (AMS) media plates, and serial dilutions conducted to determine the appropriate dilution for a target range of 30-300 colonies per plate. For the initial sample tube, add 20 ml of 0.9% saline and vortex for 5 minutes individually using a standard test tube adaptor or up to 6 at a time using a horizontal tube adaptor (SI-V506 for vertical holder). Create 1:10 dilution series from initial tube (10E0) to 10E-6. The first time a sample is analyzed, plate all dilutions to identify the target range of 30-300 colonies per plate. Plate a pure Methylobacterium positive control sample so there will be 50-100 colonies per plate.

After completion of the dilution series, plate the appropriate dilutions onto AMS agar plates in triplicate, and use a spreader to spread the cells around the plate. Use a new sterile plastic spreader for each dilution or flame a glass spreader between dilutions. When finished place plates upside down in the acrylic vacuum chamber. Apply a vacuum to create partial vacuum in the gas-tight vessel (typically −15 psig). Add high-purity methane (99.999%) to create an internal vessel pressure of 0 psig. This creates a methane:air ratio of ˜1:2.

After 10 days count and record the number of colony forming units per sample. For any plates that have no colonies place them back in the incubator and check at 14 days and then 21 days if necessary. Growth of Methylobacterium strains containing pMMO genes and positive control strains is observed, whereas no growth is observed on negative control plates.

### Example 7: Sequences of sMMO Protein Components and Genes Encoding Same

Sequences of genes from representative Methylobacterium strains that encode sMMO protein components are provided in the Tables 19 and 20 below.

TABLE 19

| Strain | SEQ ID NO | pMMO Component |
|---|---|---|
| NLS0737 | 1 | Regulatory B component |
| NLS0770 | | |
| NLS0737 | 2 | Hydroxylase beta chain |
| NLS0770 | | |
| NLS0737 | 3 | Reductase C component |
| NLS0770 | | |
| NLS0737 | 4 | Hydroxylase alpha chain |
| NLS0770 | | |
| NLS5278 | 5 | Regulatory B component |
| NLS5334 NLS5480 | | |
| NLS5549 | | |
| NLS5278 | 6 | Hydroxylase beta chain |
| NLS5334 NLS5480 | | |
| NLS5549 | | |
| NLS5278 | 7 | Reductase C component |
| NLS5334 NLS5480 | | |
| NLS5549 | | |
| NLS5278 | 8 | Hydroxylase alpha chain |
| NLS5334 NLS5480 | | |
| NLS5549 | | |

TABLE 20

| Strain | SEQ ID NO | s MMO Component |
|---|---|---|
| NLS0737 | 9 | Regulatory B component |
| NLS0770 | | |
| NLS0737 | 10 | Hydroxylase beta chain |
| NLS0770 | | |
| NLS0737 | 11 | Reductase C component |
| NLS0770 | | |
| NLS0737 | 12 | Hydroxylase alpha chain |
| NLS0770 | | |
| NLS5278 | 13 | Regulatory B component |
| NLS5334 NLS5480 | | |
| NLS5549 | | |
| NLS5278 | 14 | Hydroxylase beta chain |
| NLS5334 NLS5480 | | |
| NLS5549 | | |
| NLS5278 | 15 | Reductase C component |
| NLS5334 NLS5480 | | |
| NLS5549 | | |
| NLS5278 | 16 | Hydroxylase alpha chain |
| NLS5334 NLS5480 | | |
| NLS5549 | | |

### Example 8. Rice Field Evaluation

Mitigation of methane (CH₄) emission from the rice crop-soil system was evaluated following the application of NLS0737 and NLS0770 to rice seeds and evaluation of methane levels during the crop season. Two sites near Ita Ibate and Mercedes were used in the testing Argentina. The plots were installed and cultivated using conventional rice farming operations. Trial layouts are provided below.

TABLE 21
Macro plots with replicates. RCBD with 5 true replicates. 2 locations.

| | Individual plot | | | | | | |
|---|---|---|---|---|---|---|---|
| | Width | Length | Area | | | Plots/ | Area/ |
| Location | m | m | m2 | Reps | # Trt | location | location m2 |
| Ita Ibate | 5.78* | 20 | 115.6 | 5 | 7 | 35 | 4046 |
| Mercedes | 1.95** | 15 | 29.25 | 5 | 7 | 35 | 1024 |

*33 rows × 17.5 cm
**11 rows × 17.5 cm

TABLE 22
Treatments: Yield + Methane

| Trt | Name | Rate | Target |
|---|---|---|---|
| T1 | Untreated check | | Check |
| T2 | Rizoderma | 200 mL Premax T + | Check |
| | | 600 mL Rizoderma | |
| T3 | NLS0737.05 | 62 g/100 kg | Methane |
| T4 | NLS0770.05 | 62 g/100 kg | Methane |

### Seed Treatment and Planting Process

Rice seeds were treated in rotating drums in small batches. A photographic record of the process and final seed appearance for each treated batch were collected. Seed was treated with base fungicide+insecticide for all treatments ((Acronis (BASF)—thiophanate methyl 36.9%+pyraclostrobin 4.1% or Thiram+Carbendazim+Imacloprid). NLS0737 and NLS0770 were applied at a rate of 62.5 g in 600 ml of water/100 kg of seed for a target of 10⁶ CFU per seed. Seed was enumerated for CFU of viable PPFMs and planted within seven days of seed treatment. Both locations were planted using conventional methods on a commonly farmed varietal, IRGA 424 RI seed, at 100 Kg/ha.

Fertilizer was applied pre-plant broadcast using 60-100 kg KCl, and 100 kg/ha MAP at planting, in the seed row. Urea was applied pre-irrigation at 100 kg/ha and post irrigation at 50 kg/ha during the spike differentiation stage.

Untreated check: includes base chemical fungicide/insecticide treatments following farmer standards. Seed included professional seed treatment—all biological treatments were added as over treatments. At Mercedes seed was treated with Thiram+Carbendazim+Imidacloprid. At Ita Ibate the seed was treated with Acronis (BASF)—thiophanate methyl 36.9%+pyraclostrobin 4.1%

Commercial control: included a biostimulant/biofertilizer treatment on top of the base chemical treatment. Rizoderma (Trichoderma harzianum) was applied per recommended label rate.

### Foliar Spray:

Additional treatments were applied as foliar applications using NLS0737 and NLS0770. Conventional backpack spray technologies with a 2 meter boom were used to deliver 125 g/acre of the dried powder inoculant (˜10⁹ CFU per gram) in water at nine gallons/acre and ˜20 psig. Applications were made three times throughout the growing season at approximately 15, 35 and 50 days after sowing. Plots were split to allow randomized complete block analysis.

### Pre-Plant Soil Sampling and Crop Measurements

Soil was analyzed (0-20 centimeters depth) to determine % organic matter, % total nitrogen, NO₃, NH₄, pH, complete macro-micro nutrient analysis and cations+EC, and soil texture.

Crop measurements included early stand count at four and 20-days post first observed emergence in the field. Plant diseases throughout all crop stages were scouted using quantitative incidence and severity scales. Digital images for NDVI and other spectral indices for visual and quantitative assessment of treatment effects were collected using two drone flights at vegetative and reproductive stages with multispectral sensors. Weekly satellite images were analyzed from Planetscope satellite imagery at 3-m resolution for NDVI time series. GPS coordinates at each corner of trial polygons were used to digitize data and analyze statistical comparisons of treatment effects through different spectral indices associated with crop growth and health. Digital elevation models were included in the two field-scale trials to assess elevation effect on crop performance. Grain yield at plot and sub-strip scale were collected by hand. Grain samples containing one kilogram from each block were assessed for grain quality: % whole grain, % broken, % chalky grain. Complete daily weather information and irrigation scheduling and amounts were recorded.

### Methane Measurements:

Greenhouse gas collections methods were designed and contracted with specialists from The USDA ARS and executed by scientist under contract from EEA. INTA. Balcarce Ruta 226 km 73.5. C.C 276. C.P 7620. Balcarce, Buenos Aires, Argentina. Gas samples were collected using standard methods recognized by the ICCP following standard GC protocols. Headspace gas samples were analyzed at using dedicated gas chromatography methods on an Agilent Gas Chromatography system. Cylinder head space samples were collected between 9 am and 11 am. There were 5 samples collected per plot at 15-20-min intervals. A total of 100 samples were collected per time point, from 20 chambers installed per field. Measurements were taken six times during the crop season:
1. Early tillering. 5 days after first irrigation. approx. 20 days after emergence—expected peak
2. Mid tillering: approx. 35 days after emergence
3. Late tillering: 50 days after emergence
4. Pre flowering: 65 days after emergence
5. Flowering: 80 days after emergence—expected peak
6. Advanced grain filling (pre-maturity): 100 days after emergence

Under paddy rice conditions peak methane emissions are generally recognized between time points 5 and 6. Response variables include Methane (CH₄), Nitrous oxide (N₂O) and Carbon Dioxide (CO₂), expressed in kg/ha/day. Using the six measurements across the season, a model was fit to estimate total emission in kg/ha during the entire crop cycle.

### Methane Emission Results

Peak emission occurred between late tillering and flowering, as expected. Total emissions were strongly influenced by temperature fluctuations. Overall lower emission at ItaIbate vs Mercedes were most likely related to later planting which led to overall lower crop growth, both below and above ground. Average lower temperature & radiation during CH₄ measurements combined with significant soil texture differences (ItaIbate sandier) also contributed to the difference in both methane emissions and reduced yield.

NLS0737 and NLS0770 showed reductions in CH₄ emissions during peak rice growth at both locations in Argentina. At the Mercedes site NLS0737 reduced methane emissions 28% (151 kg/ha) and NLS0770 reduced emissions 23% (125 kg/ha). At the Ita Ibate site NLS0737 reduced methane emissions 7% (19 kg/ha) and NLS0770 reduced emissions 4% (11 kg/ha).

TABLE 23

| Ita Ibate | % | Kg/HA | Reduction |
|---|---|---|---|
| NLS0737 | 7.0 | 251 | 19 |
| NLS0770 | 4.1 | 259 | 11 |
| Avg. | 5.6 | 270 | 15 |

TABLE 24

| Mercedes | % | Kg/HA | Reduction |
|---|---|---|---|
| | 28.1 | 386 | 151 |
| | 23.3 | 412 | 125 |
| Avg. | 25.7 | 537 | 138 |

TABLE 25
Comined Locations
CH4 Emission Reductions

| | | |
|---|---|---|
| AVG | KG/HA | 76.5 |
| AVG | % | 15.6 |

Methane measurements showing patterns across dates at the Mercedes locations are provided in Table 26 below. Different letters represent statistically significant differences between treatments at late tillering and flowering stages. The decrease in methane at pre-flowering correlates with the lowest temperature of the season.

TABLE 26

| | Methane Emission Rate (kg/ha/day) | | | | | |
|---|---|---|---|---|---|---|
| | Early | Mid | Late | Pre | | |
| | tillering | tillering | tillering | flowering | Flowering | Maturity |
| Treatment | (4 DAF*) | (18 DAF) | (39 DAF) | (60 DAF) | (69 DAF) | (82 DAF) |
| Control (UTC) | 1.35 | 5.50 | 12.17 B | 5.82 | 7.54 B | 0.14 |
| Rizoderma | 0.64 | 4.45 | 9.49 AB | 5.79 | 7.51 B | 0.22 |
| NLS0737 | 0.71 | 4.23 | 8.19 A | 5.01 | 5.22 A | 0.23 |
| NLS0770 | 0.32 | 4.13 | 8.28 A | 5.58 | 7.13 B | 0.12 |

*DAF—days after flooding initiation

### Rice Yield Results

Rice yield at the Mercedes site was increased over the untreated control 17% by NLS0737 (+27 bu/acre) and 6% by NLS0770 (+9 bu/acre). Due to the delayed planting, the lower solar incidence and the late season rains, the yield from the Ita Ibate site was reduced 44% below the Mercedes site. No differences in yield by treatment were seen in Ita Ibate. At an alpha of 0.15 only the seed treated and sprayed yield from the NLS0737 blocks were considered significantly different from the untreated check. The NLS0737 and the NLS0770 seed treated yields were similar but not different from each other. The NLS0770 seed treated and sprayed blocks were lower, but not significantly different from the untreated check. The biological check blocks were similar to the NLS0737 seed treated and sprayed treatments.

TABLE 27

| | Yield | % Yield | | |
|---|---|---|---|---|
| Mercedes | (□) | (□) | Bu/Ac | lb/Ac |
| Check (UTC) | 158 a | | | |
| NLS0737ST/F | 185 b | 17.1 | 27 | 1215 |
| (*) | | | | |
| BiolCheck | 183 b | 15.8 | 25 | 1125 |
| NLS0737ST | 173 ab | 9.5 | 15 | 675 |
| NLS0770ST | 167 ab | 5.7 | 9 | 405 |
| NLS0770ST/F | 154 a | −2.5 | −4 | |
| (*) | | | | |

(*) Seed treatment followed by three folar applications
(□) Different letters represent statistically significant differences between treatments at a = 0.15

### Example 9. Evaluation of Plant Growth Enhancement and Methane Mitigation

Methanotrophic bacterial strains are evaluated for growth enhancement and methane mitigation in a simulated rice paddy ecosystem in a greenhouse. Methane gas flux from the rice paddy is monitored periodically by the closed-chamber method, and the plants are harvested at maturity to measure yield and biomass. Additional studies evaluate results of application of methanotrophic bacteria in combination with methylotrophic bacterial strains.

Rice seeds, Kitaake variety, are planted in 6 cell plug trays, 6 trays per treatment and arranged in a Random Complete Block Design (RCBD) in a greenhouse. Dried/sieved paddy soil is prepared by mixing 1 part paddy soil with 1 part 50/50 field soil/sand mixture and wetting before planting. Rice seeds are placed on the soil mixture and covered with a thin layer of sand. Greenhouse conditions are set at 30° C., 14 hour days, and 70% relative humidity with flood tables placed on the floor.

Plants are grown for approximately two weeks to an average plant height of approximately 10 cm. The four strongest plants from each tray are transplanted to 2 gallon pots containing a simulated flooded rice paddy soil, and a top down foliar spray of the bacterial strain or strains to be tested is applied. Pots containing the simulated flooded rice paddy soil are prepared as follows:
a. For “Amended soil” used in some studies, add 50 g of finely milled alfalfa hay to a 2 gallon pot without holes. No alfalfa is added for samples containing “Unamended soil.”
b. Add 1 g of 30-10-30 fertilizer.
c. Add 3 kg (approx. 1 gallon) of dry and pulverized paddy soil mixed 1:1 with 50/50 steam sterilized field soil and sand.
d. Repeat steps 2a-2c for as many pots as necessary. (30 for a typical experiment)
e. Gently mix soil and hay with gloved hands to incorporate hay evenly through soil.
f. Thin the plants to be transplanted to 1 plant per 6-cell insert by selecting the strongest plant in each cell.
g. Remove the four strongest plants from each 6 cell insert and place on the soil surface of the appropriate pot, arranged in a square with vertices halfway along the radius of the pot.
h. Fill to the surface of the root ball with an additional 1 kg of 50/50 steam sterilized sand/soil mixture.
i. Add dH2O until soil is fully hydrated, then continue adding dH2O until the soil is covered by 5 cm of standing water.

A top-down foliar spray of the appropriate bacterial preparation is applied to each pot (5 reps per treatment), and pots are randomized on a greenhouse flood table in an RCBD.

Plants are grown to maturity and water added as needed to maintain a 5-7 cm layer of water over the soil. Three weeks after transplant, another foliar application of the bacterial strain or strains to be tested is applied. Fertilizer (1.5 grams of 30-10-10 NPK) is added to each pot at 4 weeks post-transplant.

Gas headspace sampling is conducted at 3, 4, and 5 weeks after transplant as described below.
a. Sampling is done at approximately the same time of day each round (generally at 9 AM) and the sampling sequence is randomized and conducted by block.
b. Tables containing the pots are flooded to a depth of 2-3 inches.
c. An ambient gas sample (T0) is taken by withdrawing 30 ml of air from the center of the growth chamber.
d. Place a 10 gallon bucket equipped with a gas sampling port and running computer fan over the first pot to be sampled and weight down.
e. Repeat for all pots to be measured at time intervals to allow consistent sampling times for each pot.
f. Sample each pot 20 minutes after placing the gas sampling buckets using a 35 mL plastic syringe positioned into the silicon sampling port. Mix the headspace gas by gently drawing 10 mL gas into the syringe and evacuating 3× while needle is still in sampling port.
g. To obtain the sample, slowly draw 30 mL gas into the syringe from the chamber. Remove the syringe from the sampling port and push 5 mL of gas out of syringe before transferring the remaining 25 mL of headspace gas to a labeled silicon sealed 12 mL glass exetainer vial.
h. Repeat for the remaining pots. Flush the plastic syringe with ambient air 3× before drawing headspace gas from chamber to clean plastic syringe with any contamination from the previous flux chamber. Each pot should be sampled 20 minutes after its lid was originally sealed.
i. Repeat sampling at 40 and 60 minutes after sealing the pots.
j. Exetainers are labeled with unique identifiers and analyzed to determine gas content.

Plants are harvested when they reach the Hard Dough stage to determine yield and biomass. For each pot, all panicles are removed by cutting at the base. After panicles are removed, all foliar tissue is cut at the soil surface. Collected panicles and foliar tissue is dried (50° C. oven for 4-7 days) and weighed to determine biomass.

TABLE 28
Methane Analysis Results

| | Soil Amendment | | | |
|---|---|---|---|---|
| | Alfalfa Amended | | Unamended | |
| | Inoculant | | | |
| | NLS1508 | NLS1501 | NLS1508 | NLS1501 |
| | Methylcystis | Methylomicrobium | Methylcystis | Methylomicrobium |
| | hirsuta | lacus | hirsuta | lacus |
| Mean Total | 564.26 | 394.89 | 8.02 | 7.18 |
| Methane @ | | | | |
| wk 5 (kg/ha) | | | | |
| % Difference | 28.5% | −10% | −28.1% | −35.6% |
| from UTC | | | | |
| p-value | 0.75 | 0.38 | 0.03 | 0.01 |

A significant reduction in methane was observed following treatment of rice plants grown in unamended soil, a low methane environment, with both NLS1501 and NLS1508. NLS1501 treatment resulted in a non-significant decrease in methane in amended soil where high levels of methane were generated. Treatment under the same conditions with NLS1508 resulted in a non-significant increase in methane.

A more in-depth analysis of background levels of NLS1501 in root-associated (rhizosphere) soil identified a clear signal, using strain-specific qPCR, of NLS1501 related DNA in 9 of the 30 samples analyzed, with an approximate assay limit of detection of 1×10² genome equivalents per gram of soil. The average level of the 9 positive samples was 1.54×10² genomes per gram soil.

Based on these results, it was concluded that the background of NLS1501 (and closely related strains) in the GH pot assays described above to be at approximately the assay limit of detection of 1×10².

TABLE 29
Growth Promotion Analysis Results

| | | Soil Amendment | | | |
|---|---|---|---|---|---|
| | | Alfalfa Amended | | Unamended | |
| | | Inoculant | | | |
| | | NLS1508 | | NLS1508 | |
| | | M. hirsuta | UTC | M. hirsuta | UTC |
| Panicle | | 33.04 | 24.89 | 25.78 | 28.45 |
| | % Difference | 32.7% | — | −9.4% | |
| | from UTC | | | | |
| | p-value | 0.0044 | — | 0.041 | |
| Shoot | Biomass (g) | 32.36 | 25.74 | 29.09 | 29.63 |
| | % Difference | 25.7% | — | −1.8% | — |
| | from UTC | | | | |
| | p-value | 0.0043 | — | 0.74 | — |

Growth promotion of rice following treatment with NLS1508 is observed for rice plants grown in soil amended to contain alfalfa hay.

Growth promotion analysis following treatment with NLS1504 with and without the addition of Methylobacterium strain NLS7725 was conducted. Rice plants treated as described above with NLS1504 (Methylosarcina fibrata) and NLS1504+Methylorubrum populi strain NLS7725 were observed to evaluate growth promotion. The grain at observation was approximately half ripe, R8 growth stage, for plants grown in alfalfa amended soil. The plants in unamended soil were at growth stage R9, where seed is ripe and the plants are senescing. Plants that were treated with NLS1504+Methylorubrum populi and grown in amended soil were approximately 6 inches taller than the UTC plants. The treated plants had significantly more panicles, corresponding with an increase in number of tillers. The growth promotion effect was not observed in the pots with unamended soil. The plants are further analyzed at maturity to identify increases in biomass and yield. It was also observed that the application of NLS1504 and NLS7725 alleviated chlorosis of the plants grown in the amended soil containing high methane. This reduction of chlorosis, a common symptom of nitrogen limitation, resulted in dramatic growth promotion and yield enhancement for the treated plants. Methane samples were collected as described above and analyzed to evaluate reduction in methane levels.

### Panicle Dry Weight Measurements

### Alfalfa Amended Soil R8 Stage

TABLE 30

| Level | S | Least Sq Mean |
|---|---|---|
| NLS1504 + NLS7725 | A | 59.6 |
| NLS1504 | B | 35.4 |
| UTC | B | 35.0 |

### Unamended Soil R9 Stage

TABLE 31

| Level | S | Least Sq Mean |
|---|---|---|
| NLS1504 + NLS7725 | A | 29.8 |
| UTC | A | 29.2 |
| NLS1504 | A | 28.4 |

Different letters in the “S” column in the above results indicates significant differences between the treatments

Results of methane analysis and yield results at maturity for the above described experiment is shown below.

TABLE 32

| | | | NLS1504 + | | | NLS1504 + |
|---|---|---|---|---|---|---|
| Inoculant | UTC | NLS1504 | NLS7725 | UTC | NLS1504 | NLS7725 |
| Mean Total Methane @ | 317.8 | 377.9 | 319.5 | 2.75 | 1.23 | 2.16 |
| wk 5 (kg/ha) | | | | | | |
| % Difference from UTC | — | 18.9% | 0.5% | — | −55.3% | −21.5% |
| (negative value is | | | | | | |
| favorable) | | | | | | |
| p-value | — | 0.31 | 0.98 | — | 0.024 | 0.31 |
| Mean Yield | 23.4 | 21.4 | 55.1 | 23.2 | 21.5 | 23.0 |
| (Panicle Biomass) | | | | | | |
| % Difference from UTC | — | −8.5% | 135.5% | — | −7.3% | −0.8% |
| p-value | — | 0.73 | 0.0004 | — | 0.11 | 0.81 |
| Mean Shoot Biomass | 27.1 | 28.9 | 39.4 | 25.2 | 26.7 | 25.9 |
| % Difference from UTC | — | 6.6% | 45.4% | — | 5.6% | 2.8% |
| p-value | — | 0.48 | 0.0009 | — | 0.11 | 0.46 |

Linear mixed model for student's t-test: Yield, Biomass, Methane~Treatment + Block(&random)

Rice plants were treated as described above with NLS1501 alone and in combination with Methylobacterium radiotolerans strain LGP2020, deposited as NRRL-B-67892. Plants were grown to maturity and analyzed for their ability to reduce methane and enhance growth and yield. Soil used in this study was amended with 25 g alfalfa hay, down from 50 g in previous treatments. Microbes were inoculated onto the plants via a foliar spray at transplant and again 3-4 weeks after transplant. All pots were sampled for their rate of methane evolution at 3-, 4- and 5-weeks post-transplant and then grown to approximately R8, or the hard dough stage, at which time shoot biomass and grain biomass were determined. Methane production in these soil conditions was reduced when compared to rates seen in previous studies. No treatment resulted in a significantly lower rate of methane production when compared to an uninoculated control. Significant growth promotion was observed when NLS1501 was applied alone or in combination with LGP2020.

TABLE 33

| | Soil Amendment | | |
|---|---|---|---|
| | Alfalfa (25 g) | | |
| | Inoculant | | |
| | UTC | NLS1501 | NLS1501 + LGP2020 |
| Mean Total Methane @ wk 5 | 272.4 | 286.9 | 281.5 |
| (kg/ha) | | | |
| % Difference from UTC | — | +5.3% | +3.3% |
| (negative value is favorable) | | | |
| p-value | — | 0.71 | 0.82 |
| Mean Yield | 50.4 | 61.6 | 62.8 |
| (Panicle Biomass) | | | |
| % Difference from UTC | — | +18.2% | +24.6% |
| p-value | — | 0.0004 | 0.0002 |
| Mean Shoot Biomass | 33.6 | 33.6 | 35.6 |
| % Difference from UTC | — | 0% | +6% |
| p-value | — | 0.98 | 0.14 |

### Example 10. Methylobacterium and Methanotroph Treated Corn Plants Grown Under Reduced Nitrogen

Corn seeds treated with Methylobacterium or methanotroph strains are grown in a large-scale field trial under reduced nitrogen conditions to determine effects on foliar nitrogen levels and corn yield. The trial was conducted using a randomized complete block design with 4 reps per location. Methylobacterium strains LGP2019 (NRRL B-67743), LGP2017 (NRRL B-67741), LGP2020 (NRRL B-67892) and NLS0754 (NRRL B-68197), and methanotroph strain NLS1508 (NRRL B-68262) are applied at a rate of approximately 1×10⁶ CFU per seed. Fertilizer control treatments include standard 100% N, 70% N, 35% N, and 0% N. Fertilizer levels in microbe treatments include 70% N (all 5 strains) and 35% N (Methylobacterium strain LGP2019 and methanotroph strain NLS1508). Foliar tissue from the ear leaf at the R2-R4 developmental stage is sampled for micronutrient concentrations, including nitrogen, phosphorus, and potassium. Corn seed is harvested at maturity and analyzed to identify increases in seed yield.

### Example 11. Methylobacterium and Methanotroph Treated Rice Plants Grown Under Reduced Nitrogen

Rice seeds treated with Methylobacterium or methanotroph strains are grown in a large-scale field trial under reduced nitrogen conditions to determine effects on foliar nitrogen levels and rice yield. The trial is conducted using a randomized complete block design with 4 reps per location. Methylobacterium strains LGP2019 (NRRL B-67743), LGP2017 (NRRL B-67741), LGP2020 (NRRL B-67892) and NLS0754 (NRRL B-68197), and methanotroph strain NLS1508 (NRRL B-68262) are applied at a rate of approximately 1×10⁶ CFU per seed. Fertilizer control treatments include standard 100% N, 70% N, 35% N, and 0% N. Fertilizer levels in microbe treatments include 70% N (all 5 strains) and 35% N (Methylobacterium strain LGP2019 and methanotroph strain NLS1508). Foliar tissue from the ear leaf at the R2-R4 developmental stage is sampled for micronutrient concentrations, including nitrogen, phosphorus, and potassium. Rice seed is harvested at maturity and analyzed to identify increases in seed yield.

### Example 12. Evaluation of Rice Yield Following Treatment with Methylobacterium and Methanotroph Strains

Rice seeds are treated with Methylobacterium strain LGP2019 (NRRL B-67743), methanotroph strain NLS1508 (NRRL B-68262), and a combination of LGP2019 and NLS1508. The trial is conducted using a randomized complete block design with 4 reps per location. Rice seed is harvested at maturity and analyzed to identify increases in seed yield.

### Example 13. Identification of Methylobacterium Strains, Variants and Derivatives

Genomic sequences that can be used to identity and distinguish NLS0737 and NLS0770 from other Methylobacterium strains are identified by an exact k-mer analysis of whole genome sequences of over 5000 public and proprietary Methylobacterium isolates. NLS0737 and NLS0770 are closely related and may be, or originate from, a single Methylobacterium isolate. A 300 nt DNA fragment common to both isolates, but not found in other Methylobacterium strains analyzed is provided as SEQ ID NO:31. Genomic sequences that can be used to identity and distinguish NLS5278, NLS5334, NLS5480, and NLS5549 from other Methylobacterium isolates are identified in the same manner. NLS5278, NLS5334, NLS5480, and NLS5549 are closely related and may be, or originate from, a single Methylobacterium isolate. A 300 nt DNA fragment common to NLS5278, NLS5334, and NLS5480, but not found in other Methylobacterium strains analyzed is provided as SEQ ID NO:32.

Assays for detection or identification of specific Methylobacterium strains and closely related derivatives are developed using the disclosed unique genomic DNA essentially as described in WO2022076588 Example 3.

Unique genomic DNA sequences of additional Methylobacterium strains disclosed herein were identified by BLAST analysis of approximately 300 bp genomic DNA fragments using a sliding window of from 1-25 nucleotides and compared to whole genome sequences of over 1000 public and proprietary Methylobacterium isolates. Genomic DNA fragments were identified that have weak BLAST alignments, indicative of approximately 60-95% identity over the entire fragment, to corresponding fragments of a Methylobacterium of interest. Unique fragments from the various disclosed strains useful for assay development are provided as SEQ ID NOS: 33-75 as shown in the table below.

TABLE 34

| STRAIN | SEQ REFERENCE | SEQ ID |
|---|---|---|
| LGP2001 | ref3_25009 | SEQ ID NO: 33 |
| LGP2001 | ref3_25219 | SEQ ID NO: 34 |
| LGP2001 | ref1_4361220 | SEQ ID NO: 35 |
| LGP2001 | ref1_4602420 | SEQ ID NO: 36 |
| LGP2002 | ref4_930 | SEQ ID NO: 37 |
| LGP2002 | ref1_142021 | SEQ ID NO: 38 |
| LGP2002 | ref4_930 | SEQ ID NO: 39 |
| LGP2003 | ref1_86157 | SEQ ID NO: 40 |
| LGP2003 | ref1_142469 | SEQ ID NO: 41 |
| LGP2003 | ref1_142321 | SEQ ID NO: 42 |
| LGP2004 | ref1_194299 | SEQ ID NO: 43 |
| LGP2004 | ref1_194305 | SEQ ID NO: 44 |
| LGP2004 | ref1_194310 | SEQ ID NO: 45 |
| LGP2009 | ref1_153668 | SEQ ID NO: 46 |
| LGP2009 | ref1_3842117 | SEQ ID NO: 47 |
| LGP2009 | ref1_3842278 | SEQ ID NO: 48 |
| LGP2015 | ref1_135566 | SEQ ID NO: 49 |
| LGP2015 | ref1_135772 | SEQ ID NO: 50 |
| LGP2015 | ref1_169470 | SEQ ID NO: 51 |
| LGP2017 | ref1_1185955 | SEQ ID NO: 52 |
| LGP2017 | ref1_3282585 | SEQ ID NO: 53 |
| LGP2017 | ref1_4194637 | SEQ ID NO: 54 |
| LGP2018 | ref1_4871392 | SEQ ID NO: 55 |
| LGP2018 | ref1_1266930 | SEQ ID NO: 56 |
| LGP2018 | ref1_17614 | SEQ ID NO: 57 |
| LGP2019 | ref1_458355 | SEQ ID NO: 58 |
| LGP2019 | ref1_459688 | SEQ ID NO: 59 |
| LGP2019 | ref1_3158527 | SEQ ID NO: 60 |
| NLS0497 | ref1_46464 | SEQ ID NO: 61 |
| NLS0497 | ref1_85227 | SEQ ID NO: 62 |
| NLS0497 | ref1_98103 | SEQ ID NO: 63 |
| NLS0693 | ref1_622066 | SEQ ID NO: 64 |
| NLS0693 | ref1_2496 | SEQ ID NO: 65 |
| NLS0693 | ref1_2640477 | SEQ ID NO: 66 |
| NLS1179 | ref1_687571 | SEQ ID NO: 67 |
| NLS1179 | ref1_695522 | SEQ ID NO: 68 |
| NLS1179 | ref1_705877 | SEQ ID NO: 69 |
| LGP2167 | ref1_54084 | SEQ ID NO: 70 |
| LGP2167 | ref1_4816166 | SEQ ID NO: 71 |
| LGP2167 | ref1_2292077 | SEQ ID NO: 72 |
| LGP2020 | ref1_2810264 | SEQ ID NO: 73 |
| LGP2020 | ref1_322980 | SEQ ID NO: 74 |
| LGP2020 | ref1_2785241 | SEQ ID NO: 75 |

### Example 14. Identification of Methanotroph Strains, Variants and Derivatives

Sequences that encode pMMO protein components and 16S sequences can be used to identify methanotroph strains provided herein and variants and derivatives thereof are provided below.

TABLE 35

| | pMMO | Protein SEQ | Nucleotide SEQ |
|---|---|---|---|
| Strain | Component | ID NO: | ID NO: |
| NLS1505 | pMOA2 | 76 | 97 |
| NLS1506 | | | |
| NLS1508 | | | |
| NLS1509 | | | |
| NLS1511 | | | |
| NLS1505 | pMOB2 | 77 | 98 |
| NLS1506 | | | |
| NLS1508 | | | |
| NLS1509 | | | |
| NLS1511 | | | |
| NLS1505 | pMOC2 | 78 | 99 |
| NLS1506 | | | |
| NLS1508 | | | |
| NLS1509 | | | |
| NLS1511 | | | |
| NLS1512 | pMOA2 | 79 | 100 |
| NLS1512 | pMOB2 | 80 | 101 |
| NLS1512 | pMOC2 | 81 | 102 |
| NLS1512 | pMOC | 82 | 103 |
| NLS1501 | pMOA | 83 | 104 |
| NLS1501 | pMOA | 84 | 105 |
| NLS1501 | pMOB | 85 | 106 |
| NLS1501 | pMOB | 86 | 107 |
| NLS1501 | pMOC | 87 | 108 |
| NLS1501 | pMOC | 88 | 109 |
| NLS1504 | pMOA | 89 | 110 |
| NLS1504 | pMOA | 90 | 111 |
| NLS1504 | pMOA | 91 | 112 |
| NLS1504 | pMOB | 92 | 113 |
| NLS1504 | pMOB | 93 | 114 |
| NLS1504 | pMOC | 94 | 115 |
| NLS1504 | pMOC | 95 | 116 |
| NLS1504 | pMOC | 96 | 117 |

TABLE 36

| Strain | 16S SEQ ID NO: |
|---|---|
| NLS1505 | 118 |
| NLS1506 | |
| NLS1508 | |
| NLS1509 | |
| NLS1511 | |
| NLS1512 | |
| NLS1501 | 119 |
| NLS1504 | 120 |
| LGP2020 | 121 |
| Partial | 122 |
| NLS7725 | |

### REFERENCES

Green, P. N. and Ardley, J. K. 2018. Review of the genus Methylobacterium and closely related organisms: a proposal that some Methylobacterium species be reclassified into a new genus, Methylorubrum gen. nov. Int J Syst Evol Microbiol. 2018 September; 68(9):2727-2748. doi: 10.1099/ijsem.0.002856.
Konstantinidis K. T., Ramette A., Tiedje J. M. (2006). The bacterial species definition in the genomic era. Philos Trans R Soc Lond B Biol Sci 361, 1929-1940.
Lidstrom, M. E. 2006. Aerobic methylotrophic prokaryotes. In Dworkin, M., S. Falkow, E. Rosenberg, K.-H. Schleifer, and E. Stackebrandt (eds.). “The Prokaryotes. A Handbook on the Biology of Bacteria. Volume 2. Ecophysiology and biochemistry.” Third edition. Springer, New York. Pages 618-634.

The breadth and scope of the present disclosure should not be limited by any of the above-described embodiments, but should be defined only in accordance with the following claims and their equivalents.

1. A method for mitigating methane gas in an agricultural field that comprises:
(a) applying a composition to a soil, field, plant, plant part or seed, wherein the composition comprises at least one methanotroph selected from the group consisting of a Type II methanotroph comprising a pMMO2 protein, and Type I methanotroph,
(b) growing the methanotroph whereby the methanotroph uses methane as a carbon source;
wherein use of the methane as the carbon source oxidizes methane or reduces methane emissions.

2.-3. (canceled)

4. The method of claim 1, wherein the Type II methanotroph is Methylocystis hirsuta isolate comprising PmoA2, PmoB2 and PmoC2 proteins with sequences having at least 97% identity to SEQ ID NOS:76-78.

5. The method of claim 1, wherein the Type II methanotroph is Methylocystis hirsuta isolate selected from the group consisting of NLS1505 (NRRL B-68282), NLS1506 (NRRL B-68283), NLS1508 (NRRL B-68262), NLS1509 (NRRL B-68284), NLS1511 (NRRL B-68285), NLS1512 (NRRL B-68286), or a variant thereof.

6. (canceled)

7. The method of claim 1, wherein said Type I methanotroph comprises NLS1501 (NRRL B-68261), NLS1504 (NRRL B-68281), or a variant thereof.

8.-11. (canceled)

12. A composition comprising a fermentation product comprising a methanotroph strain, wherein said fermentation product is essentially free of contaminating microorganisms, and wherein methanotroph strain comprises NLS1501 (NRRL B-68261), NLS1504 (NRRL B-68281), NLS1505 (NRRL B-68282), NLS1506 (NRRL B-68283), NLS1508 (NRRL B-68262), NLS1509 (NRRL B-68284), NLS1511 (NRRL B-68285), NLS1512 (NRRL B-68286), or a variant thereof.

13. (canceled)

14. The composition of claim 12, wherein said composition further comprises a methylotroph.

15. The composition of claim 14, wherein said methylotroph is a Methylobacterium strain.

16. The composition of claim 15, wherein the Methylobacterium comprises LGP2000 (NRRL B-50929), LGP2001 (NRRL B-50930), LGP2002 (NRRL B-50931), LGP2003 (NRRL B-50932), LGP2004 (NRRL B-50933), LGP2005 (NRRL B-50934), LGP2006 (NRRL B-50935), LGP2007 (NRRL B-50936), LGP2008 (NRRL B-50937), LGP2009 (NRRL B-50938), LGP2010 (NRRL B-50939), LGP2011 (NRRL B-50940), LGP2012 (NRRL B-50941), LGP2013 (NRRL B-50942), LGP2014 (NRRL B-67339), LGP2015 (NRRL B-67340), LGP2016 (NRRL B-67341), LGP2017 (NRRL B-67741), LGP2018 (NRRL B-67742), LGP2019 (NRRL B-67743), NLS0497 (NRRL B-67925), NLS0693 (NRRL B-67926), NLS1179 (NRRL B-67929), LGP2167 (NRRL B-67927), LGP2020 (NRRL-B-67892), LGP2021 (NRRL-B-68032), LGP2022 (NRRL-B-68033), LGP2023 (NRRL-B-68034), LGP2028 (NRRL B-68064), LGP2029 (NRRL B-68065), LGP2030 (NRRL B-68066), LGP2031 (NRRL B-68067), LGP2033 (NRRL B-68068), LGP2034 (NRRL B-68069), NLS0665 (NRRL B-68194), NLS0754 (NRRL B-68197), NLS0672 (NRRL B-68196), NLS0729 (NRRL B-68195), NLS0049 (NRRL-B-68236), NLS0591 (NRRL-B-68215), NLS0439 (NRRL-B-68216), NLS1310 (NRRL-B-68217), NLS1312 (NRRL-B-68218), NLS0612 (NRRL-B-68237), NLS0706 (NRRL B-68238), NLS0725 (NRRL-B-68239), NLS0770 (NRRL B-68075), NLS0737 (NRRL B-68074), NLS5278 (NRRL-B-68186), NLS5334 (NRRL-B-68187), NLS5480 (NRRL-B-68188), or NLS5549 (NRRL-B-68189), NLS7725 or a variant thereof, or a combination thereof.

17. The composition of claim 15, wherein the Methylobacterium comprises LGP2002 (NRRL B-50931), LGP2003 (NRRL B-50932), LGP2004 (NRRL B-50933), LGP2009 (NRRL B-50938), LGP2015 (NRRL B-67340), LGP2016 (NRRL B-67341), LGP2017 (NRRL B-67741), LGP2019 (NRRL B-67743), NLS0693 (NRRL B-67926), or LGP2020 (NRRL-B-67892), NLS7725 or a variant thereof, or a combination thereof.

18. A plant, plant part or seed at least partially coated with the composition of claim 12.

19. A method for mitigating methanol that comprises:
(a) treating a rice field with a composition comprising at least one methanotroph selected from the group consisting of a Type II methanotroph comprising a pMMO2 protein, and Type I methanotroph, wherein said Type I methanotroph is a Methylomicrobium sp. or a Methylosarcina sp.; and
(b) growing the methanotroph in the field thereby mitigating methane.

20. The method of claim 19, wherein said composition comprises a methanotroph isolate comprising NLS1501 (NRRL B-68261), NLS1504 (NRRL B-68281), NLS1505 (NRRL B-68282), NLS1506 (NRRL B-68283), NLS1508 (NRRL B-68262), NLS1509 (NRRL B-68284), NLS1511 (NRRL B-68285), NLS1512 (NRRL B-68286), or a combination thereof.

21. (canceled)

22. An isolated methanotroph selected from NLS1501 (NRRL B-68261), NLS1504 (NRRL B-68281), NLS1505 (NRRL B-68282), NLS1506 (NRRL B-68283), NLS1508 (NRRL B-68262), NLS1509 (NRRL B-68284), NLS1511 (NRRL B-68285), NLS1512 (NRRL B-68286).

23. A method for selecting a methanotroph isolate capable of utilizing methane as a food source, wherein said method comprises:
a) detecting in the genome of a methanotroph isolate, one or more genetic elements, wherein said genetic element comprises a particulate methane monooxygenase; and
b) treating a field, water, plant, plant part or seed with said methanotroph isolate, and measuring green-house gas emissions.

24.-49. (canceled)

50. A method for increasing harvested yield of a plant, wherein said method comprises applying a composition to a soil, field, plant, plant part or seed, wherein the composition comprises at least one methanotroph.

51. (canceled)

52. The method of claim 50, wherein said methanotroph is a methanotrophic microbe selected from the group consisting of a Type II methanotroph comprising a pMMO2 protein, and Type I methanotroph.

53.-54. (canceled)

55. The method of claim 52, wherein the type II methanotroph is Methylocystis hirsuta isolate comprising PmoA2, PmoB2 and PmoC2 proteins with sequences having at least 97% identity to SEQ ID NOS:76-78.

56. The method of claim 55, wherein said Methylocystis hirsuta isolate is selected from the group consisting of NLS1505 (NRRL B-68282), NLS1506 (NRRL B-68283), NLS1508 (NRRL B-68262), NLS1509 (NRRL B-68284), NLS1511 (NRRL B-68285), NLS1512 (NRRL B-68286), and variants thereof.

57. (canceled)

58. The method of claim 52, wherein said Type I methanotroph is a Methylomicrobium or Methylosarcina species of NLS1501 (NRRL B-68261), NLS1504 (NRRL B-68281), or a variant thereof.

59.-66. (canceled)

67. The method of claim 50, wherein the composition further comprises a methylotroph and said methylotroph is a Methylobacterium strain.

68. (canceled)

69. The method of claim 67, wherein the Methylobacterium comprises LGP2002 (NRRL B-50931), LGP2003 (NRRL B-50932), LGP2004 (NRRL B-50933), LGP2009 (NRRL B-50938), LGP2015 (NRRL B-67340), LGP2016 (NRRL B-67341), LGP2017 (NRRL B-67741), LGP2018 (NRRL B-67742), LGP2019 (NRRL B-67743), NLS0693 (NRRL B-67926), LGP2020 (NRRL-B-67892), NLS0754 (NRRL B-68197), NLS7725 (NRRL B-68260), a variant thereof, or a combination thereof.

70. (canceled)


SEQUENCE LISTING (128 sequences)

SEQ ID NO: 1 - protein, 122 aa, Methylobacterium extorquens
MSTSQPTSQNTIFQKMDTMKFERTISHQCGVTMNDSVEARAIAEYMSEKPDVTVTYQPALIRIDGVGKLTFLMDEISEILGREMTAELFEVNASTHYGRMIRIDDNTVALFGSMEEALEYIE

SEQ ID NO: 2 - protein, 356 aa, Methylobacterium extorquens
MSEQQTEVVKSGAAGSAVFADSDSRKYRYFEPRGKRATHYEDVTVDVQPDPERYLKQDWIISFANGDGAYTKQSTAVKSSNWHAFRAPDQEWERTHYQRQSKIETMVQSVIANGRRAGAPKAYDKAWVKVLQNHLGAWKHAEFGLGTSMMQAQRYGYTQMINNATLTNSSYKLRLAQDITLYLSEIGMDIQGFDDEAGKIRWLEDPIWQDTRLAIETIMGSSDYLEQYFATNIVFEPLIGELFRSGFLMQAASPNSDFITPSVISAAEADYERNLANTIDLFHLLITDGEFASQNGALFQDWVTKHAALAHKAANTLQPIWSQPHAKPVQFSDARASSLERFSKILGELGLETPKE

SEQ ID NO: 3 - protein, 126 aa, Methylobacterium extorquens
MSPLWSILHDFLSSGEDARDIHFFYGARTEKDLFYIDKFAAITAEYPRFKFIPVLSNADEDTEWQGETGFVHQVVGTHLKRLDLGDDLDVYACGPTPMIDALTPVLFMNDVDTDRIYYDRFTPAPA

SEQ ID NO: 4 - protein, 556 aa, Methylobacterium extorquens
MSGALTLNKITNQRGMPVGEAAKRIADLGWNPSYVQEAMTFPTDYKITKAPKDPMKQVLRSYFPMQEEKDNRVYGALDAALRGDMFRNVQPRWVEWMKLFLAIIPFPEISAARAMAMVGRLAPGEELRTGFTMQMVDEFRHSTIQMNLKKWYMENYIDPAGFDITEEAFGKCYATTIGRQFGEGFITGDAITSANMYLTVVAETAFTNTLFVAMPSEAARNGDYALPTVFLSVQSDESRHVGNGHSMLMAMLKEPDNHLLLERDLRYAFWQNHAIVDAAVGTFIEYGTTNRDKNKESYAEMWHRWIFEDYYRTYMLPLEKYGIKVHHDDVQAAWDRLTKKFYVHAVAQFFSVGWPVNFWRIEAQTEKDFEWFEHKYPGWYAQFGEYWKWYEKLSHKGQKVLLFNEDVGYVYPHRCWSSLVPCLIREDIVVDEIDGKLHTFAHEIDRWTAVEAFSGEYQGRPTPAMGRFSGRREWEDCYHGWDLADAIKDLGFVRTDGKTLVPQPHLRFDNKEMWTLDDVRGHTLQSPLQMLRAMTPSERDAHLTQYRAGFSINPCN

SEQ ID NO: 5 - protein, 123 aa, Methylobacterium radiotolerans
MSVNARNASNQNIFQKMGDLSFDQTISHQCGVTMNDSVEARAIAEFMGQKPGVTITHQPALIRIDGNGELVFKMDEISEILGKEMTAEIFEVNTSTHYGRMIRIDDNTVTLFGDMEAALQYIE

SEQ ID NO: 6 - protein, 355 aa, Methylobacterium radiotolerans
MTLEAATRKSGAAGSAIFADSDSRKYRYFEPKGKRATHYEDMTVDVQPDPERYLLQNWIISFADGKGAYSKENTAALSSDWHAFRAPDQEWERTHYQRQSKIEAMVQSVIANGRKAGAPAAFDKAWVKILQNHLGAWKHVEFGLGTALMQAQRYGYTQMINNATLTNSSYKLRLAQDITLYLAEIGMDIAGFDDTAGKRHWLEDGTWQGTREAVEAIMGSTDYLEQYFATNIVFEPLVGELFRSGFLMQVASSNGDFITPPVISAAEADYERNLANAIDLFHLLATDAAHAEHNRTLFRSWVDRHVALARKAAADLQPIWSQPHSKPAQFADAKAASTERVTKILGELGLDLPQE

SEQ ID NO: 7 - protein, 345 aa, Methylobacterium radiotolerans
MSEVYTVKLNPVGVEFEVEEDETVLDAAFRQGIALPHGCKEGRCAACKCIVTDGEVEMLPYSTFALNETERDQNHVLLCRSLISSDIEVDLLNYDEELLSKSIPVRDLQGRVSRVSSLTHDIRLLEIELDQPLKFWAGQYVDITLPGAEEITRSFSMANPPSETQKLSFIIKKYPGGRFSSRLDGELDVGTPVGVKGPYGTCFRRENRGGALILVGGGSGMSPLWSILHDHIASGEERPVHFFYGARTQTDLFYLDLFAEVARSNPRFTFIPVLSHASDDAAWQGERGFVHEVVNDHLRRLAYGEDLDVYACGPEPMIEALTPVLQMNDVSSERIFFDKFTPART

SEQ ID NO: 8 - protein, 556 aa, Methylobacterium radiotolerans
MSGTLTLNKITAQKGISVGEAAKKIADLGWNPSYVQEAMTFPTDYKITKAPKDPMKQVLRSYFPMQEEKDNRVYGALDAALRGDMFRNVEPRWVEWMKLFLAIIPFPEISAARSMAMVGRLAPGEELRTGFTMQMVDEFRHSTIQMNLKKWYMENYIDPAGFDITEEAFGKCYATTIGRQFGEGFITGDAITSANIYLTVVAETAFTNTLFVAMPSEAARNGDYALPTVFLSVQSDESRHVGNGHSMLMSILKEPENHLLLERDLRYAFWQNHAIVDAAIGTFIEYGTTNRDKDKESYAEMWHRWIFEDYYRTYMLPLEKYGIKIHHDDVQTAWKRITEKHYVHKIAQFFSVGWPVNFWRIEAQTEKDFEWFEHKYPGWYAQFGEYWKWYGKLSRPGQKIVTFNEDVGYVYPHRCWSSLVPCVVREDLVTDEIDGQLHTFAHEIDRWTAVEAFANEYEGRPTPAMGRFSGRREWESVYHNWDLADAIKDLGFVRTDGKTLVPQPHLRFDNKDMWTLDDVRGYTLKSPLITLREMSPEAREAHLAEYRAGFTINPCN

SEQ ID NO: 9 - DNA, 369 bp, Methylobacterium extorquens
  source 1..369: mol_type = genomic DNA
atgtcgacatcacaacccacatctcaaaataccatcttccaaaagatggacaccatgaagttcgagcggacgatctcgcatcagtgcggggtcacaatgaacgatagtgttgaagctcgggcgatcgccgaatacatgtcggagaagccagatgtcactgtgacttatcaacctgcgctcatccgcatcgacggggttgggaagcttacgttcctgatggacgagatcagtgagatcctcggccgagagatgactgcggagcttttcgaggtaaacgcttctacgcactacggtcgcatgatccgcattgatgacaacacagttgcactgtttggcagcatggaagaggcgctcgaatacatcgagtaa

SEQ ID NO: 10 - DNA, 1071 bp, Methylobacterium extorquens
  source 1..1071: mol_type = genomic DNA
gtgagcgaacaacagaccgaagtggtgaagtcaggagctgccggctcagccgtattcgcagactctgacagccgcaagtatcgttacttcgaaccgcgcggcaagcgggcgacgcactatgaagatgtgacagtcgatgtccaacctgatcccgagcggtatctaaaacaggattggatcatctcctttgccaatggcgacggagcttatacgaaacagtctaccgccgtgaaaagttccaactggcatgcgttccgggcgccggaccaagagtgggagcggacccactatcagcgtcagtccaagatcgagacgatggtccaatccgttatcgccaacggtcgaagagctggagcccccaaggcttacgacaaggcctgggtgaaggttctgcagaaccatttgggtgcgtggaagcacgccgaattcggcctgggcacctcgatgatgcaggcccagcgctacggctacactcaaatgattaacaacgcgacgttgacgaactcgtcgtacaagcttcgtcttgcacaggacatcacgctttacctctcggaaatcggtatggatatccaggggtttgatgatgaggccggcaagattaggtggctggaggatccgatctggcaggacacgcgcctcgccatcgaaactattatgggttcttccgactatttagagcagtactttgcgacgaacattgtcttcgaaccactaattggagagcttttccgatcaggtttccttatgcaggccgcatccccaaatagtgacttcattaccccgtcggtgatctcggccgcagaggccgattacgagcgcaatctcgccaacaccattgacctattccatctgctcataacggacggtgaattcgcatcccagaatggtgcgctgttccaagactgggtcacgaagcatgcggcgctcgcgcacaaagctgccaacacgctccaacccatctggtcgcaaccccacgcgaaaccggtccagttttccgatgccagggccagctcattggagcggttcagcaagatcctcggcgaacttggcctcgaaacccccaaggagtga

SEQ ID NO: 11 - DNA, 381 bp, Methylobacterium extorquens
  source 1..381: mol_type = genomic DNA
atgtcgccgctgtggtcaatcctgcacgacttcctctcgtctggtgaggacgcgcgcgatatccacttcttctatggcgcgcgcacggagaaagacctattttacatcgataagttcgcagcgatcacagccgaataccctcggttcaaattcatacccgtgctctctaacgcggatgaggacaccgagtggcaaggggagacgggcttcgtccatcaagtcgttggaacacacctcaaacgccttgatctcggcgatgatttagacgtctacgcctgcggtccgacaccgatgatcgacgcgctaacaccggtgttgttcatgaacgacgttgacaccgaccggatctactatgaccggtttacccctgcgccggcctga

SEQ ID NO: 12 - DNA, 1671 bp, Methylobacterium extorquens
  source 1..1671: mol_type = genomic DNA
atgtcgggagcactaactctcaacaagataacgaaccaacgtggcatgccggtgggcgaggctgcgaagcgtattgcggatcttgggtggaacccgtcgtacgtccaggaagccatgacgtttcccacggactacaagatcacgaaggcgccgaaggacccgatgaagcaggtccttcggtcatacttcccgatgcaagaagagaaggacaaccgcgtatacggcgcactcgacgccgctttgcgcggcgacatgttccgcaacgtgcagccgcgttgggtcgagtggatgaagctgttcctggccatcatcccgttcccagagatctcggctgcgcgcgcgatggccatggtggggcggcttgctccgggcgaagaactgcgtaccggcttcaccatgcagatggtcgatgaattccgccattcgaccatccagatgaacctcaaaaaatggtatatggagaattatatcgatccggcgggcttcgatattaccgaggaagcattcggcaagtgctacgccacaactatcgggcgccagttcggtgagggcttcatcaccggcgacgcgatcacctcagcgaacatgtacctgaccgtcgtggccgagacagcgttcaccaacacgctgttcgtcgccatgccgtcggaggccgctcgtaacggagattacgctctgccgacggtgttcctatccgtgcagtccgatgagagccggcacgtgggtaacggccactcgatgctgatggcgatgctcaaggagcccgacaaccacctgctgcttgagcgcgacctccgttacgccttctggcagaaccacgccatcgtcgatgcggcggtcggcacgttcatcgagtacgggaccacgaaccgcgataagaacaaagagtcttacgcggaaatgtggcaccgctggatcttcgaggactactaccgcacatacatgttgccgctcgagaagtacggcatcaaggttcatcacgacgatgttcaggctgcttgggatcgcttgaccaagaagttctacgtccacgccgtcgcacagttcttttccgtcggctggccggtcaatttctggcgcatcgaagcccagacggaaaaagacttcgagtggttcgagcataagtaccccggctggtacgcgcagtttggcgagtactggaagtggtacgagaagctgtcccacaagggtcagaaggttctcctgttcaatgaggatgtcggatacgtctatccgcaccggtgctggagttctctggtgccatgcctcatccgtgaggacatcgtcgtcgacgagatcgatggcaagctgcacaccttcgctcatgagatcgaccgttggactgcagtggaagccttctcgggcgaataccaaggtaggccgacacctgcgatgggccgcttcagcggacgccgcgagtgggaagactgctaccacggctgggatcttgccgacgcgatcaaggatctcggcttcgttcggactgacggcaagacactcgtgccgcagccgcatctgcgcttcgacaacaaggagatgtggaccctcgacgatgttcgcgggcacaccctccagagcccgttgcagatgctgcgtgcgatgacgccatccgagcgtgacgcacaccttacccaataccgggctgggttctcgatcaatccttgcaactga

SEQ ID NO: 13 - DNA, 372 bp, Methylobacterium radiotolerans
  source 1..372: mol_type = genomic DNA
atgtctgtgaatgcacgcaatgcgtcgaaccagaacatcttccaaaagatgggggatctttctttcgatcagacgatctcacaccagtgcggtgttaccatgaacgacagcgtcgaggcgcgcgcgatcgctgaattcatgggccagaagccgggcgtcaccatcacccaccagcctgcgctgatccgcatcgatggaaacggcgaactcgtcttcaagatggacgagatcagcgaaattctcggcaaggagatgaccgcggagatcttcgaggtcaacacttcgacgcactacggcaggatgatccgcatcgacgacaatacggtcacgttgttcggcgatatggaagctgctctgcagtacatcgagtaa

SEQ ID NO: 14 - DNA, 1068 bp, Methylobacterium radiotolerans
  source 1..1068: mol_type = genomic DNA
atgacgttggaagccgccacacggaagtcgggcgcagccggctccgcgattttcgcggactcggatagccgcaagtaccggtatttcgaacccaagggcaaacgtgcgacccactacgaagacatgacggtcgacgtacagcccgatcccgagcgttacctactccagaactggatcatttcgttcgcggacgggaagggcgcgtactcgaaggagaatacggcggccctgagctccgactggcacgctttccgcgcgcctgatcaggagtgggagcgcacgcattatcagcgacagtcgaagatcgaggcgatggtgcaatccgtgatcgccaatggtcgcaaggccggcgctccggccgccttcgacaaggcctgggtgaagatcctccagaaccatctcggcgcatggaagcatgtggaattcgggctgggcaccgcgctcatgcaggcgcagcgctacggctacacgcagatgatcaacaacgcgaccctgacgaactcgtcctacaagctgcgcctcgcccaggacatcacgctctacctcgcggagatcgggatggacatcgccgggttcgacgacacggcgggtaagcggcactggctcgaggacggaacttggcagggcacccgggaagcggtggaggccatcatgggctcgaccgactacctggagcagtacttcgcgacgaacatcgtcttcgagccgctggtgggcgagctgtttcgcagcggcttcctgatgcaggtcgcgtcctcgaacggggacttcatcacgccgccggtgatctccgcggccgaggcggactacgagcgcaacctcgccaacgcgatcgacctgtttcacctgctcgccacggacgccgcgcacgccgagcacaaccggaccctgttccggagctgggtcgaccggcacgtcgctttggctcgcaaggcagctgccgatctccagccgatctggtcacagcctcattcgaagccggcccagttcgccgacgcgaaagcggcgtccaccgaacgtgtcaccaagatcctcggcgagctcggcctcgatcttcctcaggagtaa

SEQ ID NO: 15 - DNA, 1038 bp, Methylobacterium radiotolerans
  source 1..1038: mol_type = genomic DNA
atgagcgaggtatacaccgtcaaactcaacccggtgggcgtcgagttcgaggtcgaggaagacgagaccgtcctcgacgcggccttccggcagggcattgcgctcccacacggttgcaaagaggggcgttgcgcggcttgcaaatgcatcgtcaccgacggcgaagtcgagatgctgccctattccacgttcgcgctgaacgagacggagcgtgaccagaaccatgttcttctctgccggagccttatatcgagcgatatcgaggtcgacctcctcaactacgacgaggagctgctctcaaaatcgatcccggtccgggacctccaagggcgtgtcagccgcgtctcgagtctgacgcacgacattcgcctgcttgaaatcgaacttgatcaacctctgaagttctgggctggacagtatgtcgacatcacacttccgggagcagaggagattacgcgctcgttttccatggcaaatccgccgagcgaaacgcaaaaactctcgttcatcatcaaaaaatatcccggcggccgattctcctcccgcctcgacggagagctcgacgtcggaacccccgtcggcgtcaaaggaccctacggaacctgcttccgccgtgaaaacagaggaggagccctaatcctcgttgggggtggttcggggatgtcaccgctctggtccatccttcacgaccacatcgcgagcggagaagaacgcccagtccatttcttctatggcgcgcgcacgcaaacggatctgttctatctggatctgttcgccgaggtggcgcggtccaatccgcgcttcaccttcataccggtcctctcccacgcttcggacgacgccgcgtggcagggtgagcgggggttcgtccacgaggtcgtcaacgaccatctacgtcgtctcgcttacggggaagatctcgacgtgtacgcctgcggtccggagccgatgatcgaggcactcaccccagtgctgcagatgaacgacgtatcttcggagcggattttcttcgacaagttcacaccggcacggacgtaa

SEQ ID NO: 16 - DNA, 1671 bp, Methylobacterium radiotolerans
  source 1..1671: mol_type = genomic DNA
atgtctggaacactgacactcaacaagataacagcccagaaagggatctccgtcggagaggcggccaagaagatcgccgatctcggctggaacccctcgtacgtccaggaggctatgaccttcccaacggactacaagatcaccaaggcccccaaagatccgatgaagcaggttctgcggtcctatttcccaatgcaggaggagaaggacaatcgggtctacggggcgctcgatgcggcgctgcgcggcgacatgttccgaaacgtcgagccgcggtgggtcgagtggatgaagctcttcctggctatcattccgtttcccgagatttcggccgcccgttcgatggcgatggtcggtcggttggcgcccggcgaggagctgcgtaccgggtttaccatgcagatggtcgacgagttccgccactcgaccatccaaatgaacctcaagaaatggtacatggagaactatatcgacccggcgggcttcgacatcaccgaagaggcgttcggcaaatgctatgcgacgacgatcggtcgtcaattcggcgaaggtttcatcaccggcgacgcgatcacctcggccaacatttacctgaccgtggtcgccgagaccgccttcacgaacacgctgttcgtggccatgccgtccgaggccgcccgtaacggcgactatgctctgccgaccgtgttcctgtcggtgcagtccgacgagagccggcacgtcggcaacgggcattcgatgctgatgtcgatcctgaaagagccggaaaaccacctcctgctcgagcgcgacctgcgctacgccttctggcagaaccacgccatcgtcgatgccgcgattggcacgttcatcgagtacggcaccacgaaccgtgacaaggacaaggagtcctacgcggagatgtggcaccgctggattttcgaggactactatcgcacctacatgctcccgctggagaaatacggcatcaaaattcaccacgacgacgttcagacggcctggaagcgcatcaccgagaaacattacgttcacaagatcgcacagttcttctccgtcggctggccggtgaatttctggcggattgaggcgcagaccgagaaagacttcgagtggtttgagcacaaatatccgggttggtacgctcagttcggcgagtactggaagtggtacggaaagctgagccggcccggccagaagatcgtcaccttcaacgaggatgtcggttacgtctatccgcaccgctgctggagttcgctggtgccgtgcgtcgtgcgcgaggatctcgtcactgacgagatcgacggccagctgcacaccttcgcgcacgagatcgatcgttggaccgcggtcgaagccttcgccaacgaatacgagggacgtcccacaccggccatggggcggttcagcggccgtcgtgaatgggagagtgtataccacaactgggatctggcagatgccatcaaggatctcggcttcgtccgcacagacggcaagaccctcgttcctcaaccgcatctgcgcttcgacaacaaggatatgtggacgctcgacgatgtgcgtggatacacgctgaagagcccgctgatcaccttgcgcgaaatgtcgccggaggcgcgcgaggcgcatctcgccgaatatcgcgcgggcttcacgatcaatccctgcaactga

SEQ ID NO: 17 - protein, 279 aa, synthetic construct
  VARIANT 1..63: note = X can be any amino acid or can be absent.
  VARIANT 70..77: note = X can be any amino acid or can be absent.
  VARIANT 79..80: note = X can be any amino acid or can be absent.
  VARIANT 105..135: note = X can be any amino acid or can be absent.
  VARIANT 141..151: note = X can be any amino acid or can be absent.
  VARIANT 153: note = X can be any amino acid or can be absent.
  VARIANT 155: note = X can be any amino acid or can be absent.
  VARIANT 158..159: note = X can be any amino acid or can be absent.
  VARIANT 163: note = X can be any amino acid or can be absent.
  VARIANT 166: note = X can be any amino acid or can be absent.
  VARIANT 174: note = X can be any amino acid or can be absent.
  VARIANT 179: note = X can be any amino acid or can be absent.
  VARIANT 189: note = X can be any amino acid or can be absent.
  VARIANT 195..216: note = X can be any amino acid or can be absent.
  VARIANT 222..230: note = X can be any amino acid or can be absent.
  VARIANT 233: note = X can be any amino acid or can be absent.
  VARIANT 236..240: note = X can be any amino acid or can be absent.
  VARIANT 242..279: note = X can be any amino acid or can be absent.
  VARIANT 67: note = X can be any amino acid or can be absent.
XXXXXXXXXXXXXXXXXXXXXXXXXXXXXXXXXXXXXXXXXXXXXXXXXXXXXXXXXXXXXXXMPTXLPXXXXXXXXRXXPVRRLSWPDTARFLILVARVRLLDXXXXXXXXXXXXXXXXXXXXXXXXXXXXXXXLRLHAXXXXXXXXXXXVXRXGSXXAGDXLLXLMRRWLAXHEAIXALLPGVPEPXHVAQVXXXXXXXXXXXXXXXXXXXXXXRAILQXXXXXXXXXVPXSRXXXXXPXXXXXXXXXXXXXXXXXXXXXXXXXXXXXXXXXXXXXX

SEQ ID NO: 18 - protein, 154 aa, synthetic construct
  VARIANT 1..7: note = X can be any amino acid or can be absent.
  VARIANT 9..10: note = X can be any amino acid or can be absent.
  VARIANT 19: note = X can be any amino acid or can be absent.
  VARIANT 52: note = X can be any amino acid or can be absent.
  VARIANT 71: note = X can be any amino acid or can be absent.
  VARIANT 74: note = X can be any amino acid or can be absent.
  VARIANT 94: note = X can be any amino acid or can be absent.
  VARIANT 96..154: note = X can be any amino acid or can be absent.
XXXXXXXMXXPLRRTVQVXEDGRMNLPADMRRVLGLTGAGRVILTQDEDGIXITTAEQALKRVRSLAAPFXRGXGSVVDEFIAERRADAAREDXEXXXXXXXXXXXXXXXXXXXXXXXXXXXXXXXXXXXXXXXXXXXXXXXXXXXXXXXXXXX

SEQ ID NO: 19 - protein, 103 aa, synthetic construct
  VARIANT 2..29: note = X can be any amino acid or can be absent.
  VARIANT 42: note = X can be any amino acid or can be absent.
  VARIANT 69..72: note = X can be any amino acid or can be absent.
  VARIANT 77..103: note = X can be any amino acid or can be absent.
MXXXXXXXXXXXXXXXXXXXXXXXXXXXXPQSYALQILAIAXAMSVLGLGGVWIASRIYDRNTRRLEAXXXXRRGDXXXXXXXXXXXXXXXXXXXXXXXXXXX

SEQ ID NO: 20 - protein, 2503 aa, synthetic construct
  VARIANT 1..7: note = X can be any amino acid or can be absent.
  VARIANT 12..27: note = X can be any amino acid or can be absent.
  VARIANT 29..33: note = X can be any amino acid or can be absent.
  VARIANT 39: note = X can be any amino acid or can be absent.
  VARIANT 42: note = X can be any amino acid or can be absent.
  VARIANT 46..47: note = X can be any amino acid or can be absent.
  VARIANT 50: note = X can be any amino acid or can be absent.
  VARIANT 58: note = X can be any amino acid or can be absent.
  VARIANT 60: note = X can be any amino acid or can be absent.
  VARIANT 62..63: note = X can be any amino acid or can be absent.
  VARIANT 65: note = X can be any amino acid or can be absent.
  VARIANT 71: note = X can be any amino acid or can be absent.
  VARIANT 73: note = X can be any amino acid or can be absent.
  VARIANT 75: note = X can be any amino acid or can be absent.
  VARIANT 77: note = X can be any amino acid or can be absent.
  VARIANT 79: note = X can be any amino acid or can be absent.
  VARIANT 81: note = X can be any amino acid or can be absent.
  VARIANT 87: note = X can be any amino acid or can be absent.
  VARIANT 89: note = X can be any amino acid or can be absent.
  VARIANT 92: note = X can be any amino acid or can be absent.
  VARIANT 95..98: note = X can be any amino acid or can be absent.
  VARIANT 101: note = X can be any amino acid or can be absent.
  VARIANT 108: note = X can be any amino acid or can be absent.
  VARIANT 112..116: note = X can be any amino acid or can be absent.
  VARIANT 119..120: note = X can be any amino acid or can be absent.
  VARIANT 123..126: note = X can be any amino acid or can be absent.
  VARIANT 130: note = X can be any amino acid or can be absent.
  VARIANT 132..133: note = X can be any amino acid or can be absent.
  VARIANT 135..142: note = X can be any amino acid or can be absent.
  VARIANT 144: note = X can be any amino acid or can be absent.
  VARIANT 148..149: note = X can be any amino acid or can be absent.
  VARIANT 162..163: note = X can be any amino acid or can be absent.
  VARIANT 182: note = X can be any amino acid or can be absent.
  VARIANT 186: note = X can be any amino acid or can be absent.
  VARIANT 189: note = X can be any amino acid or can be absent.
  VARIANT 192: note = X can be any amino acid or can be absent.
  VARIANT 195: note = X can be any amino acid or can be absent.
  VARIANT 197: note = X can be any amino acid or can be absent.
  VARIANT 200..201: note = X can be any amino acid or can be absent.
  VARIANT 205: note = X can be any amino acid or can be absent.
  VARIANT 220: note = X can be any amino acid or can be absent.
  VARIANT 222..226: note = X can be any amino acid or can be absent.
  VARIANT 230..241: note = X can be any amino acid or can be absent.
  VARIANT 244..249: note = X can be any amino acid or can be absent.
  VARIANT 266: note = X can be any amino acid or can be absent.
  VARIANT 268..269: note = X can be any amino acid or can be absent.
  VARIANT 276: note = X can be any amino acid or can be absent.
  VARIANT 289: note = X can be any amino acid or can be absent.
  VARIANT 302..303: note = X can be any amino acid or can be absent.
  VARIANT 306: note = X can be any amino acid or can be absent.
  VARIANT 312: note = X can be any amino acid or can be absent.
  VARIANT 315..321: note = X can be any amino acid or can be absent.
  VARIANT 324: note = X can be any amino acid or can be absent.
  VARIANT 326: note = X can be any amino acid or can be absent.
  VARIANT 336..347: note = X can be any amino acid or can be absent.
  VARIANT 358..366: note = X can be any amino acid or can be absent.
  VARIANT 370..371: note = X can be any amino acid or can be absent.
  VARIANT 373..374: note = X can be any amino acid or can be absent.
  VARIANT 376..378: note = X can be any amino acid or can be absent.
  VARIANT 381..382: note = X can be any amino acid or can be absent.
  VARIANT 385: note = X can be any amino acid or can be absent.
  VARIANT 388: note = X can be any amino acid or can be absent.
  VARIANT 390: note = X can be any amino acid or can be absent.
  VARIANT 394: note = X can be any amino acid or can be absent.
  VARIANT 400: note = X can be any amino acid or can be absent.
  VARIANT 402..403: note = X can be any amino acid or can be absent.
  VARIANT 406: note = X can be any amino acid or can be absent.
  VARIANT 410: note = X can be any amino acid or can be absent.
  VARIANT 413..420: note = X can be any amino acid or can be absent.
  VARIANT 423..430: note = X can be any amino acid or can be absent.
  VARIANT 432..434: note = X can be any amino acid or can be absent.
  VARIANT 436: note = X can be any amino acid or can be absent.
  VARIANT 438..446: note = X can be any amino acid or can be absent.
  VARIANT 452: note = X can be any amino acid or can be absent.
  VARIANT 455..459: note = X can be any amino acid or can be absent.
  VARIANT 462..467: note = X can be any amino acid or can be absent.
  VARIANT 469..470: note = X can be any amino acid or can be absent.
  VARIANT 474: note = X can be any amino acid or can be absent.
  VARIANT 477: note = X can be any amino acid or can be absent.
  VARIANT 503: note = X can be any amino acid or can be absent.
  VARIANT 508: note = X can be any amino acid or can be absent.
  VARIANT 511: note = X can be any amino acid or can be absent.
  VARIANT 513: note = X can be any amino acid or can be absent.
  VARIANT 516..517: note = X can be any amino acid or can be absent.
  VARIANT 521..522: note = X can be any amino acid or can be absent.
  VARIANT 525..529: note = X can be any amino acid or can be absent.
  VARIANT 531..557: note = X can be any amino acid or can be absent.
  VARIANT 559..561: note = X can be any amino acid or can be absent.
  VARIANT 563: note = X can be any amino acid or can be absent.
  VARIANT 566..583: note = X can be any amino acid or can be absent.
  VARIANT 587: note = X can be any amino acid or can be absent.
  VARIANT 590: note = X can be any amino acid or can be absent.
  VARIANT 598..599: note = X can be any amino acid or can be absent.
  VARIANT 612: note = X can be any amino acid or can be absent.
  VARIANT 620..621: note = X can be any amino acid or can be absent.
  VARIANT 623..627: note = X can be any amino acid or can be absent.
  VARIANT 636: note = X can be any amino acid or can be absent.
  VARIANT 639: note = X can be any amino acid or can be absent.
  VARIANT 650..651: note = X can be any amino acid or can be absent.
  VARIANT 656: note = X can be any amino acid or can be absent.
  VARIANT 671: note = X can be any amino acid or can be absent.
  VARIANT 673: note = X can be any amino acid or can be absent.
  VARIANT 677..680: note = X can be any amino acid or can be absent.
  VARIANT 683: note = X can be any amino acid or can be absent.
  VARIANT 685: note = X can be any amino acid or can be absent.
  VARIANT 692: note = X can be any amino acid or can be absent.
  VARIANT 710..711: note = X can be any amino acid or can be absent.
  VARIANT 730: note = X can be any amino acid or can be absent.
  VARIANT 733: note = X can be any amino acid or can be absent.
  VARIANT 751: note = X can be any amino acid or can be absent.
  VARIANT 757..759: note = X can be any amino acid or can be absent.
  VARIANT 761: note = X can be any amino acid or can be absent.
  VARIANT 764: note = X can be any amino acid or can be absent.
  VARIANT 766: note = X can be any amino acid or can be absent.
  VARIANT 770: note = X can be any amino acid or can be absent.
  VARIANT 775..776: note = X can be any amino acid or can be absent.
  VARIANT 779..783: note = X can be any amino acid or can be absent.
  VARIANT 786: note = X can be any amino acid or can be absent.
  VARIANT 816..825: note = X can be any amino acid or can be absent.
  VARIANT 829: note = X can be any amino acid or can be absent.
  VARIANT 838..839: note = X can be any amino acid or can be absent.
  VARIANT 857..858: note = X can be any amino acid or can be absent.
  VARIANT 863..864: note = X can be any amino acid or can be absent.
  VARIANT 869..870: note = X can be any amino acid or can be absent.
  VARIANT 877: note = X can be any amino acid or can be absent.
  VARIANT 883: note = X can be any amino acid or can be absent.
  VARIANT 886: note = X can be any amino acid or can be absent.
  VARIANT 893..894: note = X can be any amino acid or can be absent.
  VARIANT 896..902: note = X can be any amino acid or can be absent.
  VARIANT 905: note = X can be any amino acid or can be absent.
  VARIANT 907..908: note = X can be any amino acid or can be absent.
  VARIANT 911..913: note = X can be any amino acid or can be absent.
  VARIANT 916: note = X can be any amino acid or can be absent.
  VARIANT 922: note = X can be any amino acid or can be absent.
  VARIANT 926..927: note = X can be any amino acid or can be absent.
  VARIANT 929: note = X can be any amino acid or can be absent.
  VARIANT 936: note = X can be any amino acid or can be absent.
  VARIANT 938: note = X can be any amino acid or can be absent.
  VARIANT 944..947: note = X can be any amino acid or can be absent.
  VARIANT 949..952: note = X can be any amino acid or can be absent.
  VARIANT 959..970: note = X can be any amino acid or can be absent.
  VARIANT 973..977: note = X can be any amino acid or can be absent.
  VARIANT 980: note = X can be any amino acid or can be absent.
  VARIANT 982: note = X can be any amino acid or can be absent.
  VARIANT 985..989: note = X can be any amino acid or can be absent.
  VARIANT 991..993: note = X can be any amino acid or can be absent.
  VARIANT 1006: note = X can be any amino acid or can be absent.
  VARIANT 1008..1009: note = X can be any amino acid or can be absent.
  VARIANT 1016: note = X can be any amino acid or can be absent.
  VARIANT 1019..1020: note = X can be any amino acid or can be absent.
  VARIANT 1023..1024: note = X can be any amino acid or can be absent.
  VARIANT 1026: note = X can be any amino acid or can be absent.
  VARIANT 1030..1034: note = X can be any amino acid or can be absent.
  VARIANT 1038..1039: note = X can be any amino acid or can be absent.
  VARIANT 1046..1047: note = X can be any amino acid or can be absent.
  VARIANT 1049..1050: note = X can be any amino acid or can be absent.
  VARIANT 1053: note = X can be any amino acid or can be absent.
  VARIANT 1055..1056: note = X can be any amino acid or can be absent.
  VARIANT 1058: note = X can be any amino acid or can be absent.
  VARIANT 1060..1061: note = X can be any amino acid or can be absent.
  VARIANT 1063..1065: note = X can be any amino acid or can be absent.
  VARIANT 1067: note = X can be any amino acid or can be absent.
  VARIANT 1069: note = X can be any amino acid or can be absent.
  VARIANT 1073: note = X can be any amino acid or can be absent.
  VARIANT 1076: note = X can be any amino acid or can be absent.
  VARIANT 1079..1080: note = X can be any amino acid or can be absent.
  VARIANT 1082..1084: note = X can be any amino acid or can be absent.
  VARIANT 1090: note = X can be any amino acid or can be absent.
  VARIANT 1093..1095: note = X can be any amino acid or can be absent.
  VARIANT 1097..1099: note = X can be any amino acid or can be absent.
  VARIANT 1103..1104: note = X can be any amino acid or can be absent.
  VARIANT 1107..1109: note = X can be any amino acid or can be absent.
  VARIANT 1111..1112: note = X can be any amino acid or can be absent.
  VARIANT 1114: note = X can be any amino acid or can be absent.
  VARIANT 1116..1118: note = X can be any amino acid or can be absent.
  VARIANT 1121..1122: note = X can be any amino acid or can be absent.
  VARIANT 1128..1129: note = X can be any amino acid or can be absent.
  VARIANT 1132: note = X can be any amino acid or can be absent.
  VARIANT 1136: note = X can be any amino acid or can be absent.
  VARIANT 1140: note = X can be any amino acid or can be absent.
  VARIANT 1143: note = X can be any amino acid or can be absent.
  VARIANT 1146..1148: note = X can be any amino acid or can be absent.
  VARIANT 1150: note = X can be any amino acid or can be absent.
  VARIANT 1156..1157: note = X can be any amino acid or can be absent.
  VARIANT 1159: note = X can be any amino acid or can be absent.
  VARIANT 1166..1167: note = X can be any amino acid or can be absent.
  VARIANT 1169: note = X can be any amino acid or can be absent.
  VARIANT 1172..1191: note = X can be any amino acid or can be absent.
  VARIANT 1193: note = X can be any amino acid or can be absent.
  VARIANT 1195..1197: note = X can be any amino acid or can be absent.
  VARIANT 1199: note = X can be any amino acid or can be absent.
  VARIANT 1202..1204: note = X can be any amino acid or can be absent.
  VARIANT 1206..1209: note = X can be any amino acid or can be absent.
  VARIANT 1214..1215: note = X can be any amino acid or can be absent.
  VARIANT 1218..1219: note = X can be any amino acid or can be absent.
  VARIANT 1221: note = X can be any amino acid or can be absent.
  VARIANT 1223: note = X can be any amino acid or can be absent.
  VARIANT 1228: note = X can be any amino acid or can be absent.
  VARIANT 1231..1232: note = X can be any amino acid or can be absent.
  VARIANT 1235..1236: note = X can be any amino acid or can be absent.
  VARIANT 1238: note = X can be any amino acid or can be absent.
  VARIANT 1241..1243: note = X can be any amino acid or can be absent.
  VARIANT 1245: note = X can be any amino acid or can be absent.
  VARIANT 1247: note = X can be any amino acid or can be absent.
  VARIANT 1250..1251: note = X can be any amino acid or can be absent.
  VARIANT 1253..1258: note = X can be any amino acid or can be absent.
  VARIANT 1261: note = X can be any amino acid or can be absent.
  VARIANT 1263..1265: note = X can be any amino acid or can be absent.
  VARIANT 1267..1277: note = X can be any amino acid or can be absent.
  VARIANT 1280: note = X can be any amino acid or can be absent.
  VARIANT 1283: note = X can be any amino acid or can be absent.
  VARIANT 1285..1287: note = X can be any amino acid or can be absent.
  VARIANT 1289..1291: note = X can be any amino acid or can be absent.
  VARIANT 1293..1294: note = X can be any amino acid or can be absent.
  VARIANT 1301: note = X can be any amino acid or can be absent.
  VARIANT 1305..1313: note = X can be any amino acid or can be absent.
  VARIANT 1319: note = X can be any amino acid or can be absent.
  VARIANT 1322..1325: note = X can be any amino acid or can be absent.
  VARIANT 1328..1329: note = X can be any amino acid or can be absent.
  VARIANT 1331..1452: note = X can be any amino acid or can be absent.
  VARIANT 1454: note = X can be any amino acid or can be absent.
  VARIANT 1457..1458: note = X can be any amino acid or can be absent.
  VARIANT 1464: note = X can be any amino acid or can be absent.
  VARIANT 1467..1469: note = X can be any amino acid or can be absent.
  VARIANT 1471..1472: note = X can be any amino acid or can be absent.
  VARIANT 1476: note = X can be any amino acid or can be absent.
  VARIANT 1478: note = X can be any amino acid or can be absent.
  VARIANT 1482: note = X can be any amino acid or can be absent.
  VARIANT 1485..1486: note = X can be any amino acid or can be absent.
  VARIANT 1488: note = X can be any amino acid or can be absent.
  VARIANT 1493: note = X can be any amino acid or can be absent.
  VARIANT 1497: note = X can be any amino acid or can be absent.
  VARIANT 1501: note = X can be any amino acid or can be absent.
  VARIANT 1503: note = X can be any amino acid or can be absent.
  VARIANT 1505..1506: note = X can be any amino acid or can be absent.
  VARIANT 1508..1511: note = X can be any amino acid or can be absent.
  VARIANT 1515: note = X can be any amino acid or can be absent.
  VARIANT 1524..1526: note = X can be any amino acid or can be absent.
  VARIANT 1530..1531: note = X can be any amino acid or can be absent.
  VARIANT 1534..1538: note = X can be any amino acid or can be absent.
  VARIANT 1541: note = X can be any amino acid or can be absent.
  VARIANT 1550: note = X can be any amino acid or can be absent.
  VARIANT 1552..1553: note = X can be any amino acid or can be absent.
  VARIANT 1560: note = X can be any amino acid or can be absent.
  VARIANT 1563..1565: note = X can be any amino acid or can be absent.
  VARIANT 1570..1576: note = X can be any amino acid or can be absent.
  VARIANT 1583..1589: note = X can be any amino acid or can be absent.
  VARIANT 1591: note = X can be any amino acid or can be absent.
  VARIANT 1595: note = X can be any amino acid or can be absent.
  VARIANT 1604: note = X can be any amino acid or can be absent.
  VARIANT 1607..1611: note = X can be any amino acid or can be absent.
  VARIANT 1615: note = X can be any amino acid or can be absent.
  VARIANT 1625: note = X can be any amino acid or can be absent.
  VARIANT 1635..1637: note = X can be any amino acid or can be absent.
  VARIANT 1652: note = X can be any amino acid or can be absent.
  VARIANT 1654: note = X can be any amino acid or can be absent.
  VARIANT 1656: note = X can be any amino acid or can be absent.
  VARIANT 1708..1711: note = X can be any amino acid or can be absent.
  VARIANT 1714: note = X can be any amino acid or can be absent.
  VARIANT 1735: note = X can be any amino acid or can be absent.
  VARIANT 1737..1741: note = X can be any amino acid or can be absent.
  VARIANT 1757: note = X can be any amino acid or can be absent.
  VARIANT 1761: note = X can be any amino acid or can be absent.
  VARIANT 1763: note = X can be any amino acid or can be absent.
  VARIANT 1775: note = X can be any amino acid or can be absent.
  VARIANT 1781..1782: note = X can be any amino acid or can be absent.
  VARIANT 1784: note = X can be any amino acid or can be absent.
  VARIANT 1792..1793: note = X can be any amino acid or can be absent.
  VARIANT 1809: note = X can be any amino acid or can be absent.
  VARIANT 1812: note = X can be any amino acid or can be absent.
  VARIANT 1815..1823: note = X can be any amino acid or can be absent.
  VARIANT 1827: note = X can be any amino acid or can be absent.
  VARIANT 1829: note = X can be any amino acid or can be absent.
  VARIANT 1851: note = X can be any amino acid or can be absent.
  VARIANT 1855..1858: note = X can be any amino acid or can be absent.
  VARIANT 1863: note = X can be any amino acid or can be absent.
  VARIANT 1867..1869: note = X can be any amino acid or can be absent.
  VARIANT 1871: note = X can be any amino acid or can be absent.
  VARIANT 1881: note = X can be any amino acid or can be absent.
  VARIANT 1896: note = X can be any amino acid or can be absent.
  VARIANT 1899..1900: note = X can be any amino acid or can be absent.
  VARIANT 1902: note = X can be any amino acid or can be absent.
  VARIANT 1906..1909: note = X can be any amino acid or can be absent.
  VARIANT 1920..1922: note = X can be any amino acid or can be absent.
  VARIANT 1944: note = X can be any amino acid or can be absent.
  VARIANT 1952..1953: note = X can be any amino acid or can be absent.
  VARIANT 1965: note = X can be any amino acid or can be absent.
  VARIANT 1969..1970: note = X can be any amino acid or can be absent.
  VARIANT 1973: note = X can be any amino acid or can be absent.
  VARIANT 1976..1977: note = X can be any amino acid or can be absent.
  VARIANT 1979: note = X can be any amino acid or can be absent.
  VARIANT 1992: note = X can be any amino acid or can be absent.
  VARIANT 1997..2004: note = X can be any amino acid or can be absent.
  VARIANT 2007: note = X can be any amino acid or can be absent.
  VARIANT 2012: note = X can be any amino acid or can be absent.
  VARIANT 2014..2015: note = X can be any amino acid or can be absent.
  VARIANT 2021: note = X can be any amino acid or can be absent.
  VARIANT 2049..2051: note = X can be any amino acid or can be absent.
  VARIANT 2053: note = X can be any amino acid or can be absent.
  VARIANT 2066: note = X can be any amino acid or can be absent.
  VARIANT 2076: note = X can be any amino acid or can be absent.
  VARIANT 2085: note = X can be any amino acid or can be absent.
  VARIANT 2087: note = X can be any amino acid or can be absent.
  VARIANT 2092: note = X can be any amino acid or can be absent.
  VARIANT 2096..2097: note = X can be any amino acid or can be absent.
  VARIANT 2100: note = X can be any amino acid or can be absent.
  VARIANT 2109..2165: note = X can be any amino acid or can be absent.
  VARIANT 2167..2178: note = X can be any amino acid or can be absent.
  VARIANT 2180..2186: note = X can be any amino acid or can be absent.
  VARIANT 2188..2191: note = X can be any amino acid or can be absent.
  VARIANT 2193..2209: note = X can be any amino acid or can be absent.
  VARIANT 2211..2212: note = X can be any amino acid or can be absent.
  VARIANT 2217..2218: note = X can be any amino acid or can be absent.
  VARIANT 2220..2222: note = X can be any amino acid or can be absent.
  VARIANT 2224..2225: note = X can be any amino acid or can be absent.
  VARIANT 2230..2231: note = X can be any amino acid or can be absent.
  VARIANT 2233..2235: note = X can be any amino acid or can be absent.
  VARIANT 2240: note = X can be any amino acid or can be absent.
  VARIANT 2242: note = X can be any amino acid or can be absent.
  VARIANT 2245..2246: note = X can be any amino acid or can be absent.
  VARIANT 2252: note = X can be any amino acid or can be absent.
  VARIANT 2256..2257: note = X can be any amino acid or can be absent.
  VARIANT 2260: note = X can be any amino acid or can be absent.
  VARIANT 2265..2266: note = X can be any amino acid or can be absent.
  VARIANT 2270..2271: note = X can be any amino acid or can be absent.
  VARIANT 2275..2276: note = X can be any amino acid or can be absent.
  VARIANT 2278..2279: note = X can be any amino acid or can be absent.
  VARIANT 2283..2286: note = X can be any amino acid or can be absent.
  VARIANT 2288: note = X can be any amino acid or can be absent.
  VARIANT 2290..2291: note = X can be any amino acid or can be absent.
  VARIANT 2298: note = X can be any amino acid or can be absent.
  VARIANT 2300..2301: note = X can be any amino acid or can be absent.
  VARIANT 2303..2308: note = X can be any amino acid or can be absent.
  VARIANT 2313..2322: note = X can be any amino acid or can be absent.
  VARIANT 2325..2327: note = X can be any amino acid or can be absent.
  VARIANT 2329: note = X can be any amino acid or can be absent.
  VARIANT 2332: note = X can be any amino acid or can be absent.
  VARIANT 2335: note = X can be any amino acid or can be absent.
  VARIANT 2340: note = X can be any amino acid or can be absent.
  VARIANT 2342..2353: note = X can be any amino acid or can be absent.
  VARIANT 2356: note = X can be any amino acid or can be absent.
  VARIANT 2359..2367: note = X can be any amino acid or can be absent.
  VARIANT 2370: note = X can be any amino acid or can be absent.
  VARIANT 2373..2374: note = X can be any amino acid or can be absent.
  VARIANT 2376..2406: note = X can be any amino acid or can be absent.
  VARIANT 2414: note = X can be any amino acid or can be absent.
  VARIANT 2416: note = X can be any amino acid or can be absent.
  VARIANT 2418: note = X can be any amino acid or can be absent.
  VARIANT 2423..2503: note = X can be any amino acid or can be absent.
  VARIANT 954: note = X can be any amino acid or can be absent.
  VARIANT 956: note = X can be any amino acid or can be absent.
  VARIANT 996: note = X can be any amino acid or can be absent.
  VARIANT 998: note = X can be any amino acid or can be absent.
XXXXXXXDTLEXXXXXXXXXXXXXXXXRXXXXXLACTVXDHXSIAXXQNXVPIIRDIXLXNXXDXDLADVXLXIXAXPXLXRPLTLXIXRIXAGXXXXIDXPDLRIDXAILXXXXXAGXXESXXXXVTLXLXXSXXXXXXXXEXAREXXDLRLLPPSHWGGXXAAPELLAAFVRPNDPAVDXILRXAAXILXRAXRXTAXXDGYXSGRKARAWEMAEAIXAXXXXXAMAXXXXXXXXXXXXRIXXXXXXYVLPPASFERSGQKVRXPXXIVERRLXTCLDLTLLWAACXEQAGLNPLLVLTXXHAXLGLWLXDEXXXXXXXDDXQXLRKRRDLQEXXXXXXXXXXXXLILIETTILTXXXXXXXXXDPPXXFXXAXXXGAXXIDXDAXAXLEMXLDLRRXRXXGIXPLDXGEXXXXXXXXAPXXXXXXXXLXXXQXLXXXXXXXXXAPPSFXEDXXXXXIDXXXXXXPXXRLEXWKXRLLDLTLRNKLLNFKPGKGSLTLDCXEPGAXEDXLXAGXXFRLXXRPXXXXXDXXXXXXXXXXXXXXXXXXXXXXXXXXXAXXXRXEIXXXXXXXXXXXXXXXXXXELEXRLXDLFRLARXXFEEGGANVLFLAXGFLTWTRXXGXXXXXRAPLLLVPXALXRASVRAGFRLXXHDEEXRLNPTLLEMLRQDFXLXMPDXXXXLPXDXSGIDVEXIWRIVRTHIRDLKGWEVXXEVVLSAFSFTKFLMWKDLXERXDLLKRSPVVRHLLDTPKXAYGDGXXXTXFPXPXRLDXEHPPXXIFXXXXXPLXADSSQLSAILAAASGKDFVLFGPPGTGKSXXXXXXXXXXQTIXNMIAQCLAXXGRTVLFVSQKSAALEVVXXRRRLXXVGLGXXCLEVHAXKAQKTXVIXQLREAWXXRXXXXXXXWDXAXXDLXXXREXLNGVVXSLHXXRXNGLSAHXAXGRVIAXXXXGXXXXLXLXWPXXXXXXXXXXXXSLXXXXXRAXCXELXXXXXLXXXVGXIXDHPLRGIXAXXWSPLWRXEMXXAIXXLXRTLXXXXXSGQXXAEAMGLXXLXXTYXGXXRXLXXLXXXLXRXEARXGLXFLXXGXXXLRQAVXARXXXQXXXARLXXRLXXXYXXPXVXXXDLXXLLAEWXXAKXSNFXLRGXRLXRVXXXLXPFAQGXXPXDIGPDLXXLXEIXXXXXXXXXXXXXXXXXXXXVXEXXXAXLGXXXPXXXXWSDPXXPAXXFXAXMAWAXRLXXVIXXMXPLXXXGXDXVRXXLXXXXXXLDXEXXXLXXXXXXXXXXXGGXLAXAXXXFXXXRXXAVKAIEXLGRXXXXXXXXXLAGRAXPDXXXXPVXXEXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXDXWVXXTLAVAXRWXXXLXXKAQXWXAWQXAAXXAXKAGLXPLVXAIEXGXIXXDXXXXAFEXAYARWWIDXXXTDDXXLRXXXXXFMXQRHEEAIRXFXXADSRLSXLAXXXVRARXXXXXXXIGGGVPXXXXXXXAXAFGXDPEWGTLAXEIXXXXXTKRXRHMPLRQLFXRMPNALTRLXXXTPCLMMSPLSIAQYXPXEXKPFDIVIFDEASQIAPWDAIGAIARGRQVVIVGDPEQLPPTNVGDRGVDEIXXXXDGXDVADQESILDECLAANLPQRXLXXXXXWHYRSRHESLIAFSNXHYYXGXLVTFPSPVTDDXRAVRLXXVXDGLYERGXXRVNRPEARALVAEVVXRLXDPXXXXXXXXXAFAXEXRSLGIVTFNGEQQRLIENLLDXERRXXXXPELEXFFDXXXWXEPVFVKNLEXVQGDERDAILFSVAXGPXXDXTGRXXXXISSLNREGGHXXXRRLNVAITRARRELVVFASMRXDQVDLGRXXARGVRDFKHFLXFAEXXGAXALXXAXAPTGGDIESPFEXAVMAXXXXXXXXALXARGWXIXXQVGVSXFRIDLGIVHPDAPGRYLAGVECDGATYXXXHXAATARDRDRLREXVLTDLGWRIXRVWSTDWWXDXQGALXRLDXXLRXDLDADRAKXXXXXXXXXXXXXXXXXXXXXXXXXXXXXXXXXXXXXXXXXXXXXXXXXXXXXXXXXPXXXXXXXXXXXXPXXXXXXXQXXXXPXXXXXXXXXXXXXXXXXYXXADLSXXGXXXDXXRFHDXXYXXXLAAMXAXVVXXEGPVFXDILXXRLXRAHGXXRITXXLRQXXLXXVDPXXXXTXEXXRIVLWPXGXXPXXXXXXFRPAXXXXXXXXXXRAXXXDXPLXELXGLARXLXXXXXXXXXXXXMAXRLXXXXXXXXXGLXRMXXAXXXXXXXXXXXXXXXXXXXXXXXXXXXXXXXRARFAEAXAXLXARESXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 21 - protein, 409 aa, synthetic construct
  VARIANT 1..84: note = X can be any amino acid or can be absent.
  VARIANT 105: note = X can be any amino acid or can be absent.
  VARIANT 115: note = X can be any amino acid or can be absent.
  VARIANT 130: note = X can be any amino acid or can be absent.
  VARIANT 132: note = X can be any amino acid or can be absent.
  VARIANT 143..144: note = X can be any amino acid or can be absent.
  VARIANT 153..159: note = X can be any amino acid or can be absent.
  VARIANT 173..208: note = X can be any amino acid or can be absent.
  VARIANT 220..226: note = X can be any amino acid or can be absent.
  VARIANT 238..247: note = X can be any amino acid or can be absent.
  VARIANT 260: note = X can be any amino acid or can be absent.
  VARIANT 267..269: note = X can be any amino acid or can be absent.
  VARIANT 285: note = X can be any amino acid or can be absent.
  VARIANT 298: note = X can be any amino acid or can be absent.
  VARIANT 300..301: note = X can be any amino acid or can be absent.
  VARIANT 303: note = X can be any amino acid or can be absent.
  VARIANT 305..306: note = X can be any amino acid or can be absent.
  VARIANT 320..322: note = X can be any amino acid or can be absent.
  VARIANT 324: note = X can be any amino acid or can be absent.
  VARIANT 327..328: note = X can be any amino acid or can be absent.
  VARIANT 330: note = X can be any amino acid or can be absent.
  VARIANT 333..335: note = X can be any amino acid or can be absent.
  VARIANT 349: note = X can be any amino acid or can be absent.
  VARIANT 368..382: note = X can be any amino acid or can be absent.
  VARIANT 386..409: note = X can be any amino acid or can be absent.
  VARIANT 117: note = X can be any amino acid or can be absent.
XXXXXXXXXXXXXXXXXXXXXXXXXXXXXXXXXXXXXXXXXXXXXXXXXXXXXXXXXXXXXXXXXXXXXXXXXXXXXXXXXXXXMQTILYARVSTADQTIAHQRXQAEAAGFKIXDXVVADEGVSGVSTXLXDRPQGRRLFDXXMLRRGDVLXXXXXXXVVRWVDRLGRNYAXXXXXXXXXXXXXXXXXXXXXXXXXXXXXXXXXXXXDVTETIREFMRXXXXXXXRGVIVRTVINNXXXXXXXXXXMTFDGATTDPMQXAVRDALXXXIGFMAATAQAQAEATXKEAQKAGIEHAKXRXXEXDXXAYRGRKPSYTREQXXXDXVRXXLXQGXXXVSAIAKATGLSRQXTVYRIRDNPAEAEAALARXXXXXXXXXXXXXXXWAAXXXXXXXXXXXXXXXXXXXXXXXX

SEQ ID NO: 22 - protein, 218 aa, synthetic construct
  VARIANT 2..19: note = X can be any amino acid or can be absent.
  VARIANT 23: note = X can be any amino acid or can be absent.
  VARIANT 25..26: note = X can be any amino acid or can be absent.
  VARIANT 47: note = X can be any amino acid or can be absent.
  VARIANT 51..54: note = X can be any amino acid or can be absent.
  VARIANT 56..60: note = X can be any amino acid or can be absent.
  VARIANT 62: note = X can be any amino acid or can be absent.
  VARIANT 74..130: note = X can be any amino acid or can be absent.
  VARIANT 136..140: note = X can be any amino acid or can be absent.
  VARIANT 144..145: note = X can be any amino acid or can be absent.
  VARIANT 147..161: note = X can be any amino acid or can be absent.
  VARIANT 163: note = X can be any amino acid or can be absent.
  VARIANT 166..218: note = X can be any amino acid or can be absent.
MXXXXXXXXXXXXXXXXXXYDDXIXXADAAAGEERDAIMRALAEDMXEASXXXXRXXXXXGXFVRAERPADLAXXXXXXXXXXXXXXXXXXXXXXXXXXXXXXXXXXXXXXXXXXXXXXXXXXXXXXXXXRALGRXXXXXDRRXXQXXXXXXXXXXXXXXXRXASXXXXXXXXXXXXXXXXXXXXXXXXXXXXXXXXXXXXXXXXXXXXXXXXXXXXX

SEQ ID NO: 23 - protein, 77 aa, synthetic construct
  VARIANT 1..17: note = X can be any amino acid or can be absent.
  VARIANT 21..22: note = X can be any amino acid or can be absent.
  VARIANT 40: note = X can be any amino acid or can be absent.
  VARIANT 50..51: note = X can be any amino acid or can be absent.
  VARIANT 67: note = X can be any amino acid or can be absent.
  VARIANT 72..73: note = X can be any amino acid or can be absent.
XXXXXXXXXXXXXXXXXMPVXXGIGIGRGDPLRPAVTRTXRFSGPEGFHXXPGALWLAAAAPLLATXLLLLXXRLAA

SEQ ID NO: 24 - protein, 101 aa, Methylobacterium radiotolerans
MPTAIPIRPAPERCLSWPDTARLLILVARVRILDLEMHTVVRHGSGFADDRLLHLMRRWLAQHEAISALLPGVAEPRHVAEVRAILQVPNSRPEPEDRRAL

SEQ ID NO: 25 - protein, 83 aa, Methylobacterium komagatae
MPQRRTIQVTEDGRMNLPADIRRVLGLNGAGRIVLMQDEDGIHLTTAEDPLRRVRELAAPFRRGSGSVVDEFIAERRADSGED

SEQ ID NO: 26 - protein, 48 aa, Methylobacterium komagatae
MPLDYALQITAIAFGLSVLGLGGAFIASRVYDRNTRRYDEAAQLHKAD

SEQ ID NO: 27 - protein, 1982 aa, Methylobacterium komagatae
VQDGIQITCSVTEHVSLAYHENAVPVIREVVVENTSEQELSDVRVRIESRPAVVQPLTLRIDRIPAGSNHHIELPDVRLDAALLAGFTEASRLELTVIVEDAAGERARHLEELRVLPPSHWGGGRSAPELLAAFVRPNDPAVDVVLRDAATKLGEAGRETGLNGYTTAKKSRAWELAEAIWAAIADRRIAYVLPPASFERAGQKVRGPSDVLERKVGTCLDLSLLYAACLEQAGLNPVLVLTVGHAFVGVWLQDDDFASATVDDMQLLRKRRDLQDLVFVETTLLTPEPPATFKVATTQGGVQVEDEAPAALEIAIDVRRCRRRGIRPMDLGDGKPTGIAPAPTIPLNQTLSAPPSFEEEARAPVDEAPETPVGRVERWKRKLLDLTLRNKLLNFKPGKGSVSLECASPGALEDGLAAGTEYRLKPLSDVLTGSDERSADLYARRHHDDGRRSYLEAALARKEIYTTSTEADLDRRLLDLYRLARNGFEEGGANILFLAVGFLSWTKKEGEAAYRAPLLLVPVTLKRSSVRAGFKLALHDDEVRINPTLLEMLREDFKLRMPELEGDLPRDGSGYDVDGIFRIVRQHVKELRGWEVVPDVVLSAFSFTKYLMWKDLVDRAEVLKRNPVVRHLIDTPKHSYGDGTPFPEPTRLDREHPPETVFAPLSADSSQLSAVLAAAGGKDFVLFGPPGTGKSQTIGNMIAQCLAQGRTVLFVSQKTAALEVVQRRLQEIGLGDYCLEVHSTKAQKSAVLGQLRRAWHERSTPSQGTWDAATSELASLREELNGLVNALHRRRENGLSAYEAFGRVIASGGEAPLVLTWPDHLAHNETTLANLRAACRELRPVLASVGSLVDHPLQGVEATQWSPVWRDDMGAAIRAVEQTLGALRVSGQAFAEAIGLPSLLATYAGTRGLVVLGNYLVRSEARCGAAFLADGAGDLRRAVAARERFQTTKVQLLGRLTGRYRPGILDQNLGALLAEWVAAQGANFLVKGGKLKKVSAQVQPYAEGPLPPDLGPDLTGLIEVARHVKAGCLEELILARLGLPWSNPDCPASEFASAITWAEKVEQLLDILGPLSLGIDGLRDHLVHLVERQGRALADGGRIAQTYAAFAQDRARANEAMKALGVLAGRPDPEEPLAAEADWIERSCTIARRLSSGLSRAQGWCAWQAAAQSALKTGLAPLIDALEDGRIAPDRAEIAFEIAYARWWIDRVVSDDPVLRRFLPARHEDAIQRFRAADARVTELSKQVVRSRLGGGIPGATAFGADPEWGTLSHELTKKTAHMPLRKLFGKMPTALTKLTPCVMMSPLSIAQYLPPDKEPFDVVIFDEASQISPWDAIGALARAKQVVIVGDPEQLPPTNVGDRGVDDIEDGSDVTDQESILDECLAANIPRRNLDWHYRSRHESLIAFSNSRYYGGRLVTFPSPVTDDRAVRLTLVPDGVYKRGSGRVNRPEARAVVADIVRRLRDPSFSEERRSLGVVTFNGEQQRLIENLLDEQRRSYPELEPFFDRDRWHEPVFVKNLENVQGDERDAIIFSVAVGPDQTGRPVSTVSSLNKDGGHRRLNVAITRARRELVVFASMRPEQIDLGRTRARGVRDFKHFLEFAERGARALAEAFAPTGGDVESPFEAAVMAGLEARGWTVHTQIGVSGFRIDLGIVHPDAPGRYLAGVECDGATYHSSATARDRDRLREHVLTDLGWRIRRVWSTEWWMDAEGALTKLDQRLIEDLEADRAKAAAAAAEAPRDVAVEPEAVEQEHDEPTGEPEVTPPVDTGPSEPANDLEPVTDLIPQRLYADQALPVTPPAPKPEVYDDVRAYRIVDLNDLGRSVEPGRFYDASYQQALSAMVDHVLAVEGPIYEELLIKRIARAHDIQRVGPLVREAIADRIDASVARTEDDGRPVLWPRGEEPRASYPHRPASAAIRSHTDTPMPELVGIAMTLPSNASEAERARMIGQRLGLSRIEASARARFERASELARQAAVA

SEQ ID NO: 28 - protein, 210 aa, Methylobacterium radiotolerans
MSVVLYARVSTAEQTLEHQQTQAEAAGFVFDAVVADHGESGRKPLRDRPEGRRLYDMLRTGDVLVVRWINRLGRSYEDVTGVMRELMQRGVIVRTIISNMTFDGATKDPMQRAIRDALIAFMAAAGEAELEATREAQKAGIEHARKQADQTAYRGRKPSYTRDQLTVISGMLGRGAGVSAIAAETGLSRQTIYRVQADPVEAEAALARWA

SEQ ID NO: 29 - protein, 69 aa, Methylobacterium gregans
MLSLDDIAAAAAGEERDALWRSLVEDMEEAAGRRRGGRGLVQADRPADLARALGRDRRVQPSRLARSAS

SEQ ID NO: 30 - protein, 54 aa, Methylobacterium radiotolerans
MPVGIGIGRGDPLRPAVTRTARFSGPEGFHPGALWLAAASPLLATLLLLVRLAA

SEQ ID NO: 31 - DNA, 300 bp, Methylobacterium extorquens
  source 1..300: mol_type = genomic DNA
aaggcccctcaatataaggagagattccctaccggagtgacgtttcactgcgcaagttatacagattgcaaaaatcacattggtagcacctactccccaagctacgccaatctctccaactaacggccctaccagaacagcagctactcggccaacgcttagtactgcagtagtaaggaatatcgctctgccatttccatttgcgagcaatacggcaccacataagctgaaaatagcgttaactgcataaaacggtattaatacttgcactaacgccgttgctgagacccatttttcccc

SEQ ID NO: 32 - DNA, 301 bp, Methylobacterium radiotolerans
  source 1..301: mol_type = genomic DNA
cgcgccaagcgatcctccggcgagacatccagcgcgcgatcctccgacacgtcgaagggatgcccgttgatcgaggatcgggccttgtggcggatggctgggtaatccgccttgatcttcgctaggtcctccgcgctcatagcgccattgcgcgccggtacactgtaattggccgtccgctggaacaccgtgagatgggcggccttcgccgcgatcaccggagcggcttggatcccggtggaaccagtcccgatcaggccaacccgccggcccgtgaaatcgacatcctcatggggccatc

SEQ ID NO: 33 - DNA, 300 bp, Methylobacterium radiotolerans
  source 1..300: mol_type = genomic DNA
gcccttctgtcaggcgatattgtataatggcgttgccccaatagaagcagccattcgtgcgagggcagcagcgacgctaggtcgaaagagcatcctaatctcgatcaagatgcgactgagatttctgatgaaaatatctagacacaagcaaagctggtgaaattacaacgatcatggcgacaattgcggccaattcggccggaacttgaaggaacataaaaatgaatattacaaatataccgcaaagcatgtagagttgctacaccaagggtcgggacgtccaaaaaaactcactgagga

SEQ ID NO: 34 - DNA, 300 bp, Methylobacterium radiotolerans
  source 1..300: mol_type = genomic DNA
ggaacataaaaatgaatattacaaatataccgcaaagcatgtagagttgctacaccaagggtcgggacgtccaaaaaaactcactgaggaagtcgactggaagcacgaggcgccccccccaggagcggggcgaccggcaagggggcccgcaattgtcgccatgatcgaccagcttaggtaggatcctctttcgacctaacgaatggctgcttctattggggcaacgccattatacaatatcgcctgaccatctggaacgcggcccggtccaccggcaggttggcgacgacagcgtcggag

SEQ ID NO: 35 - DNA, 300 bp, Methylobacterium radiotolerans
  source 1..300: mol_type = genomic DNA
cggcgtcgaccagccgggcgaactgcttgggcatgctctcccgcgacgccggccacagccgcgtccccgtccctccgcacaggatcatcgggtggatttgaaaggcaaaacgggacatcaggataggccgctcaggcgttggcgctgaggcgcttgatgtcggcgtcgaccatctcggtgatcagcgcctcgaggctggtctcggcctcccagccgaaggtcgccttggccttggcggggttgcccagcagcacctcgacctctgccggccggaacagcgccgggtcgacgatcaggtgg

SEQ ID NO: 36 - DNA, 300 bp, Methylobacterium radiotolerans
  source 1..300: mol_type = genomic DNA
ctggacatgcgcccaccccggccaagtccgaccgcaccggcaaccgctcctgtagtcgtcgtcatcgttctcacccctgaggcggagaccgtccgctaacggggtgtctcaagcaaccgtggggcggaggaacacgcacgtagtcgcgtttcaaggttcgcacgaacgcctcggccatgccgttgctctgcgggctctccagcggcgtcgtttttggcaccaaaccaaggtcgcgggcgaagcggcgcgtgtcgcggggactgtcaggaatttcgtgtgggggcggccatagtggatccg

SEQ ID NO: 37 - DNA, 300 bp, Methylobacterium radiotolerans
  source 1..300: mol_type = genomic DNA
gcaaaacgacctaatagttctacagcggcatgcgccaagtcagcgcggtgaacagtatacctgggagcaacttgtcctccgaaacccacataaaacaaattactcctggcagtgcccagtccatcaaaatcgaatacaatatttctcgaggaggcatctgtaatagcctgccaaagcaacaaagctatggcgccgttatgactttcattgcttctggtagacataaaataatatgccgatttgtgatcccaaatgtagaatattgccgcatcaattgcgccaagtttatttcggatcgat

SEQ ID NO: 38 - DNA, 300 bp, Methylobacterium radiotolerans
  source 1..300: mol_type = genomic DNA
ggcgccaacggtatgatcgcatgattttcctgcggcatagcttgcgggaatggcgtatttggcgctctcctcaggaatttctaagggcatacgcaggaactctacagcacttttactggtattttgtagtgacagcggaggaggctggtgctcaaggtaatcgtgatgaagtgatccgggccattcggggcgcgtttctagtctttccaatccgcgccctgtaccacgtattacgccggaccggtctgcgccgcgccgccctcttgaccgccctaaatgtctaagagcgtctaacaaagc

SEQ ID NO: 39 - DNA, 300 bp, Methylobacterium radiotolerans
  source 1..300: mol_type = genomic DNA
gacgatatcgctcatcttcactgcattgaagctggtgccgtactgcatagggatgaaaaagtgatgcggatagacggctgacgggaaagcgcctggtcgatcgaagactttgctgacgaggttgtggtagccccggatataggcatcgaaggccgggacgttgatcccatcctttgccttatcttgactggcgtcgtcgcgtgccgtcagaacgggcacgtcgcaggtcatcgaggccagcaccttgcggaacacctgcgttccgccgttgggattatcgacggcgaacgcggtggccgc

SEQ ID NO: 40 - DNA, 300 bp, Methylobacterium extorquens
  source 1..300: mol_type = genomic DNA
agcccacaagcctgatgcacttaactacatcctctaatgtcgcgccaatttgcttggcggcaggggatgttgtatcgtcataggcttgtctaaccggaacttgtttgccaatctctttggcgatcgcaaccgccatctcgtgttcgtcaaccatgtgcgcgttcctctaattgcactcatggtgccacgtgcacctccgatcgtctcgtgtctagaatgaaggtgggaacaaccttacacaggctttcgcgacgcgcgaatttctggtttctccgcctcggatgtgggtttgagcgcttc

SEQ ID NO: 41 - DNA, 300 bp, Methylobacterium extorquens
  source 1..300: mol_type = genomic DNA
cttttcatttgtcatgatctcgaccaaggtattcacggcaagctcggtctgttgcttagcaagtgcctgaacttcgcgaacgatcggctctcgacccttcgggttcgagacctgtcccttttgaaaaccacgtgccctacacttttcgggatcaaggtgcgggttggctttggtcaaaattctctggcgtcccattacacgccctccgcatcatcgttcccgcgaacgatctgacccccgacttccgcgaggaagcgtgtggcgtgatcctcgaagcggaatgccacctcgaactgttcc

SEQ ID NO: 42 - DNA, 300 bp, Methylobacterium extorquens
  source 1..300: mol_type = genomic DNA
cagcagcaagcagatcgttgaaaaccgcttgaaccgcatcttgatcgggaccggaaccaatcaggtcatctaggtaaaccgagacgtaaactcgtttgcgctcggcatctttcagaacgtccgtgatgccagaccgcattagtaccatcgtcgccaaggcgggcgactgaacgaagccgatcggcagagagtaacggggaccgcccctaatcgggttgcgaacgcaagaccacttagcaaaggttcgagcacggccgaacttcgcatggtggagagccgcggcaacacggttccgtgata

SEQ ID NO: 43 - DNA, 300 bp, Methylobacterium populi
  source 1..300: mol_type = genomic DNA
ggaaatcggcttcaagtacgacgtcacgccggccatgcaggtcacgggtgcactgttcaatctcgagcgcgacaaccagccgttcccctcgaacgtggagtccggcctcgtccttggcgcaggtcagacacgcacccagggcgcggaaatcggcctggccggctatctaaccgattggtggcaggtctttggcggctacgcttataccgaggcacgcgtactctcgccactggaagacgatggagacgtgatcgcagcaggtaatctcgtcggcaacgttccgctaaatactttcagtct

SEQ ID NO: 44 - DNA, 300 bp, Methylobacterium populi
  source 1..300: mol_type = genomic DNA
cggcctggccggctatctaaccgattggtggcaggtctttggcggctacgcttataccgaggcacgcgtactctcgccactggaagacgatggagacgtgatcgcagcaggtaatctcgtcggcaacgttccgctaaatactttcagtctgttcaacaagttcgatatcaacgagaatttctccgttgctctgggctattactatcaggatgccagctttgcctcctcagacaatgcagtgcgtttgccaagttattcgcggttcgatggcgggttgttctatcgattcgacgagttgac

SEQ ID NO: 45 - DNA, 300 bp, Methylobacterium populi
  source 1..300: mol_type = genomic DNA
acgttccgctaaatactttcagtctgttcaacaagttcgatatcaacgagaatttctccgttgctctgggctattactatcaggatgccagctttgcctcctcagacaatgcagtgcgtttgccaagttattcgcggttcgatggcgggttgttctatcgattcgacgagttgacacgcgttcagcttagcgtcgagaacattttcgacaggcgttacatcatcaactccaacaacaacaacaacctcacgcctggcgcgccgagaacagtccgcgtgcaattgatcgctcggttctaaa

SEQ ID NO: 46 - DNA, 300 bp, Methylobacterium gregans
  source 1..300: mol_type = genomic DNA
tagacattccaacaaaccggcaagaggctcgtcctcactcgaggatttgttgggacttgcatgatgtcgaagcggagccgttatgacctgggtgcgatcatgcgccgagcatgggagatggctcgggaggcggcattcgcggttggcgagcgggcacggactcaccttgctgccgcgatgcgcagcgcgtgggccgaagccaagttggcactcgcgcccacgaagacggagcaggatcgtctctctccgagcgacatgatcggacatgaggacgcctaccaaggccgggttctaaaatat

SEQ ID NO: 47 - DNA, 300 bp, Methylobacterium gregans
  source 1..300: mol_type = genomic DNA
aagatggatacgacaagcgcgattacattatttgcgaaatagatggacaaataaaagacaaaggactgatgtatttccttaaatctggacaagttgacctctttcacatagaagtcaccactccctttgggacaatttggtgtcacgaaaacatagaggccgaacttcttagctgaattatcgcgctccgggttcttatgcggctgagtgaagcgcgggacagcttgcgagcagggccgccaatggcagccgggatgacacaatgctcggtctcccgacgcttcttcaatcgggagcgct

SEQ ID NO: 48 - DNA, 299 bp, Methylobacterium gregans
  source 1..299: mol_type = genomic DNA
agctgaattatcgcgctccgggttcttatgcggctgagtgaagcgcgggacagcttgcgagcagggccgccaatggcagccgggatgacacaatgctcggtctcccgacgcttcttcaatcgggagcgcttcgcagcccggggcggcgcgctcatgcgtcacgacctgggccctgcgcaccttcgcggccccgccgtcccggcagatccctgatgccccaagtgggcggccactccatcaaagaaccccggcctgtggcagatctcgtaggcataccgaggttccgcagtgcccccacc

SEQ ID NO: 49 - DNA, 300 bp, Methylobacterium komagatae
  source 1..300: mol_type = genomic DNA
acggtcaccccacggactgggcgagtacctcaccggtgttctatcataacgccgagttagttttcgaccgtcccttatgcgatgtaccaccggtgtcggcagccgatttcgtcccaccgggagctggcgttccggttcagaccaccatcatcggtcacgatgtctggattggacacggggccttcatctcccccggcgtgactataggaaacggcgcgatcgtcggggcccaggcggtcgtcacaagagatgtcccaccctatgcggtagttgctggcgtccccgcgaccgtacgacgat

SEQ ID NO: 50 - DNA, 300 bp, Methylobacterium komagatae
  source 1..300: mol_type = genomic DNA
ccaataaaagcgttggccgcctgggcaacccgatccgagcctaagactcaaagcgcaagcgaacacttggtagagacagcccgccgactacggcgttccagcactctccggctttgatcggataggcattggtcaaggtgccggtggtgatgacctcgcccgccgcaagcggcgaattactcggatcagcggccagcacctcgaccaagtgtcggagcgcgaccaaagggccacgttcgaggacgtttgaggcgcgaccagtctcgatagtctcatcgtcgcggcgaagctgcacctcga

SEQ ID NO: 51 - DNA, 300 bp, Methylobacterium komagatae
  source 1..300: mol_type = genomic DNA
cgatggcaccgacctgccatgcctctgccgtccgcgccagaatggtaaagaggacgaagggggtaaggatcgtcgctgcagtgttgagcagcgaccagagaagggggccgaacatcggcatcaaacctcgattgccactcggacgcgaagcgcgtcttgaaggagggatggaagcgaaacggccgcagagtaaccgccgacgaaagattgcacccctcatcgagcaggatcggaggtgaaggcaagcgtgggttattggtaagtgcaaaaaatataatggtagcgtcagatctagcgttc

SEQ ID NO: 52 - DNA, 300 bp, Methylobacterium radiotolerans
  source 1..300: mol_type = genomic DNA
agtcattgatcaagcaacccctattgagttggatatcgaaggatcaaggtcgcgtcaatagatgcatctatcaggccaaatgtcgcttttcaagaatggctctttcgaagctatctttataatcgctcgccattctctcattaccaaaatcgaccttaactagctcgacattgatgcgagcagctccggcaaacgaggagagattgaccttaaaggaattgaacgcctcaagcaattcagacacattaccaggagtgctatagcaacaaccagacccatatcggtcaataacctctttta

SEQ ID NO: 53 - DNA, 300 bp, Methylobacterium radiotolerans
  source 1..300: mol_type = genomic DNA
cgcaaaacgatttatcactgccatcttgttgtttgataacccttttttaccagacgttatgctgggcgagaaagaggactagcagatcggagcggtatcgcgatttttcggtagttcgcgcctacaacaggataagatccgatagtgaagcaacatggctgttttttgatttgtaagtcagcaacttaagcagccagcctatctgccgtcgcagacgcttgaggcatcgggcagcatcttagaaaaggtggcagtaattgccacagcggaacgtagcggcacggataagcacgcagggtc

SEQ ID NO: 54 - DNA, 300 bp, Methylobacterium radiotolerans
  source 1..300: mol_type = genomic DNA
cccatctggacccaatatccccttcatcgacaattcccgagtaagtgtgggttcgaggatttcgcgaaacagccttgttcgttcctccggccttaaaattggcgtgccgtcgggagatcgataggcatcccttacctgcctttcgaccgccggcacacgcgcgccggtcgtcgtgttcacggccacggaatggacgaaggtgcgccgctcatttcgctcgtttgccgtctccaccatccaggaggccagcaggacggtttcgtctcgaccgccggtcacacacaccgcaagggactcagg

SEQ ID NO: 55 - DNA, 300 bp, Methylobacterium komagatae
  source 1..300: mol_type = genomic DNA
acctgctaaaatcacgtcctctcagattgaaaaatcattgaagaaacgtgtcgaacgattgccggggattatgacgttagatcaattgaaaaatacaagctttgaaattgagttacagccaaaagatgccccggatccggacccatcagacttcggtggctagttcgagccaaactcgaacgtcgccatggcgcgcaagtcgcaataccatttcacagcgcagcggttatttcgttgtacactgtagcaatgcgtcggcttgcgcgcttccgctggcgatcaaaggtccgccgatttacg

SEQ ID NO: 56 - DNA, 300 bp, Methylobacterium komagatae
  source 1..300: mol_type = genomic DNA
tcccgaacatacaatggaggaagcgtgtggtaggccaatttgtaacgaaatatggcatcggtcacggctctctcaataaattcgatctcaagtcttctgaacgagcatgcctcatccttatcctgagcgaacgcctgccagtttgcagtcattccaacatacatagccaaaaaggcgaggtagaccttcatacgggcacctcaatcgtccccattcgttcaagctccttcaagataacagccgcaccacattgctgagatcgaagattcggatcaaatattccatcaaatttatactttc

SEQ ID NO: 57 - DNA, 300 bp, Methylobacterium komagatae
  source 1..300: mol_type = genomic DNA
gcatcctttgcgctcgcaggcctaaggtcaagcccggttacttcgtttggtagaacgaggtagacgatgcctagtcttaaggtggcccatgttaaccaacagggccagaacatgattatagttccgttagatgccaacttcggttacaaaaccgatggtgagcagtccgacatcatgttcgaaatacaggacgcggcgcggtccgccggtcttgcgggtgccgtagtagcgttctggcagtcaggtggacaaacccgtttccggggcccggctccgtggcacccattccttcgcagcctc

SEQ ID NO: 58 - DNA, 300 bp, Methylobacterium gregans
  source 1..300: mol_type = genomic DNA; mol_type = genomic DNA
caactatgtagacccgacggtgcgatttcacttcgcaaagccgcagggcagcacccttgcgctcaatgttgacgccagcgtgatctatactattaccgtcacgcacacgcagggcggcgtacagattcatcgcgagagtaagaaccaccatcagaccatcacgcgcagcgacctgagcaagcagttcggcgttggtgtggccgaccagctgacgcgcgatcaggtcatgaaggtgatcgagtcggcatttcgcgacgctacccgctaagatcggcgcccacgaaacgctacgagactagg

SEQ ID NO: 59 - DNA, 300 bp, Methylobacterium gregans
  source 1..300: mol_type = genomic DNA
agccggcatcttgttcaaggcgctcacctcgacgccgacgctgtaggcgacttgagagggcgtctcatatgaacgaagcatcttcgcgtagagaaccttcttgttctcctgcgtgatgttcgctttgcagacgttgactgccgccatgaacgccgaagccttgcgcgcttcatcgtaatcgcctgcgaaggcgggtagtgaaaagcttagtgcaatggcaaacacagccgccgaacgtcgcatggtatccgtccccgattgacggcagtgccgccatatctcggctttagcagagctgat

SEQ ID NO: 60 - DNA, 300 bp, Methylobacterium gregans
  source 1..300: mol_type = genomic DNA
aacctgcgccggccgaggtttcgcgagccgtcgccacgggcaacgcctcgcccgcgatgtgcaaaaaagtccccggcacttcgcgccgtcgtccgatccacgaccgcgaatttctcaacgagtacaaggtgcttatgggagatccgagcgtccgtcccggagcccgagaccgcgcggcccgagtaataggcgaaaaagactcctactcctcgggcttctcgggccccctcagcaacatctacgcttgccgcccatcaccctggcgggagatcagcgacgagacacaggcccacttcgccc

SEQ ID NO: 61 - DNA, 300 bp, Methylobacterium radiotolerans
  source 1..300: mol_type = genomic DNA
ctcaacgttgccgccttaaccgaagactaactaacataaatttcagttagccgcgaaacagaatatgatacttggtcttatacgacgcatagaggcgcattccgtcaagtcgcgcattgcagagtccccccgaaaattcgttttctgggagcgagcagaaacgtgtgcacgtgttctgtcactctattttctggtttagagcaatcatctcgcggctcagatcccgtcgttccatagagtggtttccggttcagtatgaattgtgagcagcccctgcggctatatctacttcgacgtata

SEQ ID NO: 62 - DNA, 300 bp, Methylobacterium radiotolerans
  source 1..300: mol_type = genomic DNA
accaacaggcagaagaggcttacaaaaatgctctagatatctataatcgcgccggtctggataataggcgggaaaagtcaaatattcttatcggacttggcgatgcagctagcgcactgagcaaatttatagacgcgaaaaatttctacagtgaaggtttagcagtccgagcgcggtcatgacggtgcttgcggactagtgtctgctcgcaattcagagcagccgcttcggctggttcagtttgggtatgccctttctgtaccagcctgtgactggtctggtatgtagcccttgaccaat

SEQ ID NO: 63 - DNA, 300 bp, Methylobacterium radiotolerans
  source 1..300: mol_type = genomic DNA
atgaagctccgtcgtgtttatgtaaagaacgttcgcagtttctacgatgctgaagaactcattctggacggagatatttctattattataggcccaaatggtggaggaaaaacaaatctacttgatgcgacgatacacttgttgagaagacatttattgcaatcgtgggctgtcgttcgccatacatacacgccaataaactattccgatcacttccaaccaaatgatcaaatcacaaactatgcgctggagaagtacagcggtcgcgaaagcgaagaccaagtcgttgagttcgatatc

SEQ ID NO: 64 - DNA, 300 bp, Methylobacterium komagatae
  source 1..300: mol_type = genomic DNA
cgggggcggtctcctcatatatggagggaattaatctgaagggtcggagcccccgctacggcacacacacggcggcgggcggtgggctccttcctcatatacgcttcctgataatttcgaccgtaatcgcccgccggccgagcggccagaccttattagtccagcatcacacggtgcatcgtatcgtggagggcgaacaaccgcctaaggttcgccacgtagccatccacatccgcccttgccgctcgcgcaagggaagcagaatgcttgatagcgacttcgcgggcgtctttggtagcc

SEQ ID NO: 65 - DNA, 300 bp, Methylobacterium komagatae
  source 1..300: mol_type = genomic DNA
tgtgggcgctcatggaaacgggggacgtgcttttttgatctctagccattgtcatcatccatcggtccgggtgacaggcctttacgcacataacaacggttatttcgatcagccccctcctgatcgcacaagtcagctatacagatgcttctgaaagccaacaaaggcgttacgtatctgcgtgaccccaccgagctgcgctgcggcatcgctgacagctctgtattttaattcgaggaggtggggcaatttagcctggtcaagctcgccaacgtcttccttcacatattgagacaggac

SEQ ID NO: 66 - DNA, 300 bp, Methylobacterium komagatae
  source 1..300: mol_type = genomic DNA
agagtgtatttccccaagactttatctctttgctcatcacaaaacagcccgctcaagatgatatgcgctgggatttgtgccgcgacccgttgtaattgtatttacctctcggctcatgttccctgtcgccggagcgattggaggtaaagtaatctcaactttgcggcccatggctgatgctagctcagctacgcgacctaaggtaatatctttgtgtccgcggagttcgcgatgaatgaccgagcgattgacgccgatggtgcgggcaatatcggcttgagttagaccgttcttggcttc

SEQ ID NO: 67 - DNA, 300 bp, Methylobacterium bullatum
  source 1..300: mol_type = genomic DNA
acctgtctcgacatcgggggcagccacgaggcgtcggccgtggtagatctcggcgaagcggtagtagtgggcgaggtccccatcgggatcgatgggggagtccggcgtaccttctccctcttctgcgattagcgacagggctcgcatggcgctgggtgcgtcagtgatggcgaacaacctactatcggggaaccaccgcctgtcgacgagctggcggtccgggtttcctttgaagacagcgtcacccagcgcttcgatcttctcaatcaaggctgcgtagaactgcccgatggtcgcgaa

SEQ ID NO: 68 - DNA, 300 bp, Methylobacterium bullatum
  source 1..300: mol_type = genomic DNA
gacgtggtgacggtggcaccagtcgcccagcgaagcggctactgtactgagcccgcagtcatgtcggctgaagaaatggtcgatatgccccagggaggtcgggcgtaggtcgtacctcaaattcaccggcaggatcttgtccagcaaggcgtccgaattcgcatcgacctgacttaggaaattaccgaggtaatcgctcggtccgttcgcgtcggcggcctgcatcgccagcagcgccttatgctcggtcgtcaactcacgagcgaatattgtccaggacaggttgtcgacctcggcgac

SEQ ID NO: 69 - DNA, 300 bp, Methylobacterium bullatum
  source 1..300: mol_type = genomic DNA
gagcatggcaggccaccacgcatcgcttcttcatcaccttgctcggacgacttgctgctttgtcggtctctctctcaaggacgagacgctgcgacacttgcttcgtcagagtgccaggattaatcccggtcaataccactactacgtcagctggcgtgcgccgggcggcacgagggacgtcgacgcggaacgattgattcgagacgctaactttgacgtgtacaatttagtcacgcttttccttagcgacgccgagatagcctcgttgggccgcttgctcaccgccgatgcagccgagtt

SEQ ID NO: 70 - DNA, 300 bp, Methylobacterium dankookense
  source 1..300: mol_type = genomic DNA
aacgcgatgggagcagcacgtaatgggcgtcatacaagatataaaagaaggacttgaaggtagaatagataaccttaaggaaaatattgataatatcagttccagtatcacatgcacaatacaaggagtgccgaaggtcgagtcagaaagaattttgggatgtgcaaacgcttcgtttgaccaaatacagaaaatgtggatgaatggtcctacggatagggctgcatttctcgacgccgtgaaaagtttgatagaccgcaacgcaagtaatggaaaattcggagttccaggatcatattg

SEQ ID NO: 71 - DNA, 300 bp, Methylobacterium dankookense
  source 1..300: mol_type = genomic DNA
ttttcctttcgcgcctatgacctgagtgcggccgaagctgtccgcttattagagttttacaaccagatgcgagcggcttagctcttcctccgttattaagcagggcgcctcttcttaggggcgccccttcatatttaatcttgtctgatgtctggcgccatatcagacaaacatcagcgttgaccttgattttgcatctacattagggttgctcccagaatggagccgccgatgtccgtcccgtacctcaagcgagagatgtggggtgtgtattacatccattggcgcgagggcggccgc

SEQ ID NO: 72 - DNA, 300 bp, Methylobacterium dankookense
  source 1..300: mol_type = genomic DNA
gttggagatggtgccgtttgcaaccgtcagatcgcctactgtgaatccggtgacctcccgagagaacgtgaaggtaactgtcgtagtctcgccaactgccaaagaggtgtccgcaacggcgatggtcgctgtcggggcgcttgtttggacatcaacggtgagacccgccgaggcaggccccgtgttaccagcggtatcgacggccctagcggtgaccgtgtgcgatccagccgttagcgtcgaactggtgatgctataggtcccgccgatcgccactgccgagcccagaatggtcgtgcc

SEQ ID NO: 73 - DNA, 300 bp, Methylobacterium radiotolerans
  source 1..300: mol_type = genomic DNA
accgaaggcgtccccggacacgaaggcctgaaacaccatatctgtggcgatcaggccgacgtggtcgcggacttcaactggcagagaatgccaggccgcttcgatttcagatgatactggtacggacataggagcggcttagctttctcagtgcaaatgtgattgattccggctcaaaaatgatcttgatcggacgagacgttttcaatccatgtcgtgttgccatcgccgatcggtgcgtcaagagacagatggcgccgaccgtagatacgcgttcgggttgcccgcaccgcttctcca

SEQ ID NO: 74 - DNA, 300 bp, Methylobacterium radiotolerans
  source 1..300: mol_type = genomic DNA
ggaggtgtgatctgatgatgtgctggatgaaattggcggtcgagcacttgttcagcttggccagctcgacgagatcggcgtgatgctcggcgtcgatcaggatgttcagcgagaccggacgtacgcaggacttggtattagcgccgttgcgcatcagcttgcagccttgctctgcttctcagcgtgccgcgtcaggatgaccctgatgtagctgttgaggttgatgccgtaatagcctgcggactctgtgagatcccggcgaagatcgtcggcgagggtcaggcggatggtgctggtcgg

SEQ ID NO: 75 - DNA, 300 bp, Methylobacterium radiotolerans
  source 1..300: mol_type = genomic DNA
aagtaaccgctcaacatgatcttcagcatgttgtccaacagcaggagaatacatgtaattcaccatgaccggcaagctgcgactggccattgcttccaccgcttgaatgtagcgatcgaatttcgcaaaatcagggtggaatgaaaatatcgaaccaaactgcgagccttgaatccgttctgcaaaattatcgaaaaattttcttggccgactgccgttcgaaaacattcttacgtttacatgcggcccgcctgaaacaagacagtctaccagctctgggaaatgggggtgaagggtcgg

SEQ ID NO: 76 - protein, 256 aa, Methylocystis hirsuta
MSASTETGSPAGKTWKSKEEFLGCVILTDWILLVILFAVLLGSFHIHYMLLAGDWDFWIDFKDRRMWPTVTPIVAMCFAAAAQSFFWTRFRLPIGATTVVLALLIGEWINRYDNFWGWTFFPINLVFPSALIPMGFWLDIVLMISGSWLVTALLGGLGWGLLFYPINWPVLAQYHQATEIDGVLLTLADLIGFNYVRTGTPEYIRMVERGTLRTFGKDVVPVAAFFSGFISMLVYFLWWNMGKWFSTTKYIEIEEV

SEQ ID NO: 77 - protein, 429 aa, Methylocystis hirsuta
MKSLNLQSMAKCATARVVRLWILGLAVAGALTTLPAATPAAAHGERSQQAFLRMRTLNWYDVKWSKTELNVNDEMELTGKVHVFSGWPQAVARPGESFLNVGEPGPVLIRKSAFVGEVPVPRTFSMDVGEDYEYKIVLKARRQGRYHVHVQINVKDGGPIVGPGQWITIKGDMKDFTNPVTLLEGSTIDLETYGITWTYIYHFFWMAAATAWILYWFMTKGIIVRYWQVKAGKGRELIGQDEKRIGAITLAAVLLAVLVFYAAANREFPRTLPMQAGLLTGISPINEPRSTVAVQFKGGSYTVPGRELRIRAKVTNNGKEPVRLGEFTAAGLRFLNPEVFTTRPDFPDYLLADRGLSVSDPNPIAPGETRDLEVIIQDARFDIERLSDLAYDTDSQFGGLLFFFTPSGERQRAEIGGPVIPKFQAGATL

SEQ ID NO: 78 - protein, 259 aa, Methylocystis hirsuta
MMNQTTEKAVSAAQAGETDTIVNFKPAYITMGVMLVFYVGIRIYEQYFGWKAGLDSFAPEFQTYWMNLMWTELPLEFVAFCAIGGYLWKTRDRNIDAVAPREEMRRILTLIGWLAVYAFSVYWGASYFTEQDGTWHQTVIRDTDFTPSHILEFYLSYPIYIICGWGAFMYAHTRIPQFAKRISLAFLMFFAGPFMIFPNIGLNEWGHTFWFMEELFTAPLHWGFVFFGWFALAVFGVARQVLDRVIELSKEYEKDALAL

SEQ ID NO: 79 - protein, 256 aa, Methylocystis hirsuta
MSASTETGSPAGKTWKSKEEFLGCVILTDWILLVILFAVLLGSFHIHYMLLAGDWDFWIDFKDRRMWPTVTPIVAMCFAAAAQSFFWTRFRLPIGATTVVLALLIGEWINRYDNFWGWTFFPINLVFPSALIPMGFWLDIVLMISGSWLVTALLGGLGWGLLFYPINWPVLAQYHQATEIDGVLLTLADLIGFNYVRTGTPEYIRMVERGTLRTFGKDVVPVAAFFSGFISMLVYFLWWNMGKWFSTTKYLEIEEV

SEQ ID NO: 80 - protein, 429 aa, Methylocystis hirsuta
MKSLNLQSMAKCATARVVRLWILGLAVAGALTTLPAATPAAAHGERSQQAFLRMRTLNWYDVKWSKTELNVNDEMELTGKVHVFSGWPQAVARPGESFLNVGEPGPVLIRKSAFVGEVPVPRTFSMDVGKDYEYKIVLKARRQGRYHVHVQINVKDGGPIVGPGQWITIKGDMKDFTNPVTLLEGSTIDLETYGITWTYIYHFFWMAAATAWILYWFMTKGIIVRYWQVKAGKGRELIGQDEKRIGAITLAAVLLAVLVFYAAANREFPRTLPMQAGLLTGISPIDEPRSTVAVQFKGGSYTVPGRELRIRAKVTNNGKEPVRLGEFTSAGLRFLNPEVFTARPDFPDYLLADRGLSVSDSNPIAPGETRDLEVIIQDARFDIERLSDLAYDTDSQFGGLLFFFTPSGERQRAEIGGPVIPKFQAGATL

SEQ ID NO: 81 - protein, 259 aa, Methylocystis hirsuta
MMNQTTEKAVSAAQAGETDTIVNYKPAYITMGVMLVFYVGIRIYEQYFGWKAGLDSFAPEFQTYWMNLMWTELPLEFVAFCAIGGYLWKTRDRNIDAVAPREEMRRILTLIGWLAVYAFSVYWGASYFTEQDGTWHQTVIRDTDFTPSHILEFYLSYPIYIICGWGAFMYAHTRIPQFAKRISLAFLMFFAGPFMIFPNIGLNEWGHTFWFMEELFTAPLHWGFVFFGWFALAVFGVARQVLDRVIELSKEYEKDALAL

SEQ ID NO: 82 - protein, 257 aa, Methylocystis hirsuta
MSVTTQSAAGALDRAEPIVELKGMWIGLAVLNGFYLVVRIYEQIYGWRAGLDSFAPEFQTYWMSILWTEIPLELISGIGLAGWLWKTRVRDMSTVTPRQEMKAIVDNAAWLVVYAAAIYWGASFFTEQDGTWHMTVIRDTDFTPSHIIEFYMSYPIYSIMAIGCFFHAKTRIPYFAKGYSIAYLIVSIGPFMIIPNVGLNEWGHTFWFMEELFVAPLHWGFVFFGWMALGVFGVVIQLLLNIHRVIGKDGVELLTAA

SEQ ID NO: 83 - protein, 250 aa, Methylomicrobium lacus
MSLPITTQSIKPYAGEKARLVRAYDYLILVLALFLFIGSFHLHFALTVGDWDFWVDWKDRQWWPLVTPLMGITFPAAVQAVLWDKFRLPLGATLCVAALLIGTWVTRIFAYHYWNFFPVNMVLPATMLPGALVLDAILMLSNSFTITAIVGGSAFALLFYPSNWPIFALFHMPVEYANAQLTLADLFGFQYIRTGMPEYLRIIERGTLRTYGQYAAPLAAFCSALLCTLMYALWWYIGKWFATVRYLNKI

SEQ ID NO: 84 - protein, 247 aa, Methylomicrobium lacus
MSASQSAVRSRAEAVKVSRTFDWLICFTLFFIILGGYHVHFMLTGGDWDFWTDWKDRRLWVTVLPIVGITFPAAVQAVLWYRYRLPFGAMLAVLGLLFGEWVNRYFNFWGWTYFPVNFVFPSQFVPGALVLDVILMLSNSMQLTAVLGGLAYGLLFYPGNWPVIAPLHVPVEYNGMVMTLADLQGYHYVRTGTPEYIRMVEKGTLRTFGKDVAPVSAFFSGFVSIIIYFLWHFFGRWFGSTAFTQSS

SEQ ID NO: 85 - protein, 408 aa, Methylomicrobium lacus
MKIKMTKWVCAVGLTVMGLCSPLQTALAHGEKALEPFIRMRTIQWYDVAWSKQQFKVNEEVAVTGRFHVAEDWPVSVPKPEATFLNISTPGPVLIRTERYLNGKPWVNSVALQPGGDYEFKVVLKGRLPGHYHIHPFFNLKDAGQVMGPGVWLDISGTAGDFNNEVRTLNGEIVDMESVGLLNGIGWHLFWGVLATAWLLWWVRRPLFISRYRMLQAGMEEELITANDRLIAKAVLVAVPVAVLAINAVTVNRYPNAIPLQASLDRILPLSALVNAGQVDAEAVKAEYVVAKRAMIFTVRINNSSDKPVQIGEFNTANASFINPELPAAVSQASAIGATLRLDDNTPIRPGEERTVTVTAQDGLWETEKLDGLINDADSRVGGLLFLYASEGGERYISSVSAAVIPKF

SEQ ID NO: 86 - protein, 414 aa, Methylomicrobium lacus
MKLIKDKVAKLSFVALLLTVAAAMFYTPTASAHGEKSQAAFMRMRTIHWYDLNWSKEVVKVNDTMTISGKFLVFGGWPETVDKPESAFLNIGIPGPVFIRAGSWIGGQLVPRSVSLELGETYEFKVLLKARRPGDWHVHTMMNVEGGGPIIGPGKWVTVEGSMSQFTNPITTLTGQTIDLETYNLGNTYFWHAFWYAIGAAWMIYWMRKPMFIPRLIAVEAGKADTLITATDKKVALAFGAATIGIVAFTMAQTNSEYPITTPLQAGLLRGMKPLDLPTPTVSVKIDDATYRVPGRSMNMTLTVTNNGDTAVRLGEFNTAGVRFLDAGVTEDTTGYPDDLLAPEGLTVSDNSPLAPGQTRTIQVNASDAAWEVYRLADLIYDPDSRFAGLLFFHDENGNKQTVQIDGPLIPTFI

SEQ ID NO: 87 - protein, 257 aa, Methylomicrobium lacus
MATPSELMPVVTPTGHRLPWYLQDLPRYLKTFGALTVMYIGLRLYQGAFAVSAGLDSTEPAFEQYWMRLFYIELAVIAAVMTGLWAYLWLTRDRQLDKLAPKEEIRRYFTLTMWISIYTFAVYWAGSYFAEQDNSWHQVAIRDTPFTANHIIEFYFNFPLYVILGGCAWLYARTRLPLYAQGISLPLTLAVFGPFMILVSVGFNEWGHTFWFREEFFAAPIHWGFVIGVWFALGVGGILLQGVTRLIELLDKIEDAE

SEQ ID NO: 88 - protein, 250 aa, Methylomicrobium lacus
MAATTESVKADAAEAPLLNKRNLFLGVAIYNVFYAWVRWYEGVYGWSAGLDSFAPEFETYWMNFLYIEFVLEVSTAGILWGYIWKTRDRKVMSITPREELRRHFTHWTWLVCYSVAIYFGASYFTEQDGTWHQTIVRDTDFTPSHIIEFYLSYPIYIITGGASFLYAKTRLPTYQQGLSLQYMVSVVGPFMILPNVGLNEWGHTFWFMEELFVAPLHYGFVFFGWAALGALGVLNIECQAIAKLLKKDLA

SEQ ID NO: 89 - protein, 250 aa, Methylosarcina fibrata
MAANLTKQIFRPYTGEKARITRAYDYLILVLALFLFIGSFHLHFALTVGDWDFWIDWKDRQWWPLVTPLMGITFPAAVQAVMWEKFRLPFGATLCVAALLIGTWITRVFAYHYWNYFPINMVLPATMLPSALVLDVILMLSNSLTITAIFGGGAFALLFYPSNWPIFAMFHEPIESGNGQLTVADLFGFQYIRTGMPEYLRIIERGTLRTYGQYAAPLAAFCSALLCSLMYTLWWYIGKWFATVRYLAKI

SEQ ID NO: 90 - protein, 247 aa, Methylosarcina fibrata
MSTTSLSIGFDKETATVSRIYDYLILVLAGFLYIGSYHLHFMLTVGDWDFWLDWKDRQYWPLVTPVLMIAWPAAVQSVLWTHFRLPMGATLSIVGLMLGMWITRVTAYQVWTNYPLNFVLPATMIPSALVLDAILMLSPSFLVTAIIGGASFALLFYPTNWPIFAMFHVPIEWQGGLQTIADQFGFEYLRAGMPEYLRIIERGTLRTYGQMAAPLSAFFAALLCILVYAAGWYIGKAFATVRYLKRV

SEQ ID NO: 91 - protein, 247 aa, Methylosarcina fibrata
MSASQSAVRSRAEAVKVSRTFDWLICFALFFTILGGYHVHFMLTGGDWDFWTDWKDRRLWVTVLPIVGITFPAAVQAVLWWRYRLPFGAVLSVLGLLFGEWVNRYFNFWGWTYFPVNFVFPSQFVPGAIVLDVILMLSNSMQLTAVIGGLAYGLLFYPGNWPVIAPLHVPVEYNGMVMTLADLQGYHYVRTGTPEYIRMVEKGTLRTFGKDVAPVSAFFSGFVSIIIYFLWHFFGRWFGQQTFTSQT

SEQ ID NO: 92 - protein, 412 aa, Methylosarcina fibrata
MKNLLIKSVFSLSLVLSLYALNIQTVMAHGEKALEPFIRMRTIQWYDVQWSRQKFAVNEEISVSGKFHVAEDWPVSVPKPDAAFLNISTPGPVLIRTERYLNGKPYVNSVALQPGGDYSFKVVLKGRLPGRYHIHPFFNLKDAGQVMGPGVWLDVTGDASDFSNEAKTINGEVINMETYGLGNGIFWHIFWGAFASAWLLWWVRRPLFIPRYRMLQAGLEDELVTRQDKHIGKAILIGVPLFVLAANWMAIDQHPKAIPLQAALDQILPLSAQVNAGVVDVDTLKAEYRIPERAMTFLVKVNNRSDKPVQVGELAVANVRFLNKEFAQPGQETTDRMVAPDGLTLSDATPIQPGEERTMTIIAKDALWESERLDGLIRDSDSRIGGLLFFYDGLIGERFISSITAAVIPKFD

SEQ ID NO: 93 - protein, 414 aa, Methylosarcina fibrata
MKLIKDKVAKLSFVALLLTVTAAMFYTPTASAHGEKSQAAFMRMRTIHWYDLNWSKEVLAVNDTMTISGKFLVFAGWPETVDKPESAFLNIGIPGPVFIRAGSWIGGQLVPRSVSLELGETYEFKVLLKARRPGDWHVHTMMNVEGGGPIIGPGKWVTITGSMSSFTNPITTLTGQTIDLETYNLSNTYFWHAFWYAIGIAWMVYWMRKPMFVPRMIAVDAGKADSLITGTDRKVALAFAAGSIAIVAFTMQSTNSQYPVTTPLQAGLLRGMKPIEMPTPTVSVKVDDATYRVPGRSMNMTLTVTNNGDSAVRLGEFNTAGVRFLDANVTEDTTGYPDDLLAPEGLTVSDNSPLAPGQSRTIQVTASDAAWEVYRLADLIYDPDSRFAGLLFFHDESGNKQVVTIDAPLIPTFI

SEQ ID NO: 94 - protein, 257 aa, Methylosarcina fibrata
MATTSEQLSSSVENDNKVPWYLLDLPRYLKGFGVLTVIYIGLRLYQGAFAVSSGLDSTEPAFERYWMRLFYIELGVIAVFVTGIWGYLWFSRDRHLDTLSPREEIRRYFTLTMWISIYTFAVYWAGSYFAEQDNSWHQVAIRDTPFTANHIIEFYFNFPLYVILGGSAWLYARTRLPLYAKGISLPLTLAVFGPFMILVSVGFNEWGHTFWFREEFFAAPIHWGFVIGVWFALGVGGILLQGVTRMIELLDELEEKP

SEQ ID NO: 95 - protein, 262 aa, Methylosarcina fibrata
MVTAPDKIMAPSSTETYGSQWYLKEFKRYAVTFLAILFMYVSITIYQYTFGAATGLDSTDPLFEEYWMNLFYIEVIFQLILITALGYYFWKNRDLHLDQLSPREELRRYLNLTMWISIYTFSVYWAGSYFAEQDNAWHQAAIRDNIFTANHVVEFYLCFPFYTIMGISSWLYARTQLPLFAKNISLPLTLAVAGPFMIFVAVGFNEWGHTFWFREELFGAYMHYAFVIGVWFALAVGGILLQQVMRMSELIELINQDNSAHP

SEQ ID NO: 96 - protein, 250 aa, Methylosarcina fibrata
MAATTESVKADAAEAPLLNKRNLFLGVALYNVFYAWVRWYEGVYGWSAGLDSFAPEFETYWMNFLYIEFVLEVSTAGILWGYIWKTRDRKVMSITPREELRRHFTHWIWLVCYSVAIYFGASYFTEQDGTWHQTIVRDTDFTPSHIIEFYLSYPIYIITGGASYLYAKTRLPTYQQGHSIAYMVAVVGPFMILPNVGLNEWGHTFWFMEELFVAPLHYGFVFFGWAALGVLGVLNIEVSALSKLLKKDLA

SEQ ID NO: 97 - DNA, 771 bp, Methylocystis hirsuta
  source 1..771: mol_type = genomic DNA
atgtcggcatcaactgaaacaggctcgccagccggcaagacctggaaatcgaaagaagagtttctcggctgcgtcattctgacggattggatcctgttggtgatcctgttcgccgtgctgctcggctccttccacatccactacatgctgctcgccggcgactgggacttctggatcgacttcaaagaccgacgcatgtggccgacggtgacgccgatcgtcgccatgtgcttcgccgcggccgcgcagtccttcttctggacgaggttccgactgccgatcggcgccacgaccgtcgtgcttgccctgctgattggcgagtggatcaaccgctatgacaacttctggggctggaccttcttcccgatcaatctggtgtttccgtcggcgctcattcccatgggcttctggctcgacatcgtgctgatgatctcgggcagctggctggtcacggcgcttctcggcggccttggctggggcctcctgttctatccgatcaactggcctgtgttggcgcaatatcatcaggccactgaaatcgacggcgtgctgctcacgctcgccgacctcatcggcttcaactacgtccgcaccggcaccccggaatatatccgcatggtcgagcgcggcacgctgcgcaccttcggcaaggacgtggttccggtcgccgccttcttctccggcttcatttcgatgctcgtctacttcctgtggtggaacatgggcaaatggttttcgacgacgaaatatatcgagatcgaagaggtctag

SEQ ID NO: 98 - DNA, 1290 bp, Methylocystis hirsuta
  source 1..1290: mol_type = genomic DNA
atgaaaagcttgaatttgcaatcgatggccaaatgcgcgacggcccgtgtcgtccgcctttggatcctcggattggccgttgcaggcgcgctaacaaccttgcctgcggcgacgcccgccgccgcgcatggcgaacgttcccagcaggccttcctgcgcatgcgcacgctcaactggtatgacgtcaaatggtcgaagaccgagctcaacgtgaatgacgagatggagctcaccggaaaggtgcatgtgttttccggctggccgcaagccgtcgccaggcccggcgaatcatttctcaacgtcggcgagccgggaccggtgctcatccgcaagagcgccttcgtcggcgaagtgccggtgccgcgcaccttctcgatggatgtcggcgaggactatgaatacaagatcgtcctgaaagcgcgccgacaaggccgctatcacgtccatgtccagatcaacgtcaaggacggcggcccgatcgtaggtccgggccagtggatcaccatcaagggcgacatgaaggatttcaccaatcccgtgacgctgctcgaaggttcgacaatcgatctcgaaacctatgggatcacctggacctatatctatcacttcttctggatggctgccgccaccgcctggattctctattggttcatgaccaaagggatcatcgtccgatattggcaggtcaaggccggcaagggccgcgagctgatcggtcaagacgaaaagcggatcggagcgatcacccttgccgcggttctcttggctgtgctcgtcttctatgccgccgccaaccgggagtttccgcgcacgctgccgatgcaggcaggcttgctgaccggcatctcgccgatcaacgagccgcggtcgaccgtcgccgttcagtttaagggcggctcctatacggtgccgggccgtgaattgcggatcagggcgaaggtgaccaataatggcaaggagccggtccggctcggcgaattcactgccgccggtctgcgcttcctcaatccggaagtcttcacgacgcggccggatttccctgactatctgcttgccgaccgcggcctttccgtgagcgatcccaacccgatcgcgcccggcgagactcgcgacctggaagtgatcattcaggacgcccgcttcgacatcgaacgcctgtccgatctcgcctatgacaccgatagccagtttggcggactgctgttcttcttcaccccctctggcgagcgtcagagagctgaaatcggcggcccggttattccgaagttccaggcgggagccacgctctaa

SEQ ID NO: 99 - DNA, 780 bp, Methylocystis hirsuta
  source 1..780: mol_type = genomic DNA
atgatgaatcaaacgacagaaaaagccgtgagcgccgcacaagccggcgagaccgatacgatcgtgaacttcaagcccgcctacatcactatgggcgtgatgctggtgttttacgtcggcatccgaatctatgagcaatatttcggatggaaggccggcctcgattccttcgcgccggaattccagacctactggatgaatttgatgtggaccgagctgccgctcgagttcgtcgccttctgcgccatcggcggctatctctggaagacgcgcgaccgcaacatcgacgccgtcgcgccgcgcgaggagatgcgccgcatcttgacgctcatcggctggctcgcggtttacgccttctcggtctactggggcgcctcttacttcaccgaacaggatggcacctggcaccagacggtgattcgcgacaccgacttcacgccaagccacatcctcgagttctacctcagctatccgatctacatcatctgcggctggggcgccttcatgtatgcgcacacgcgcatcccgcagttcgccaagcgaatctcgctcgccttcctgatgttcttcgccgggccgttcatgattttcccgaacatcggcctgaacgagtggggccacactttctggttcatggaagagctgttcaccgccccgctgcactggggcttcgtgttcttcggctggttcgcgctcgccgttttcggcgtcgcgcgccaggtgctcgatcgcgtgatcgaactcagcaaggaatacgagaaggacgctctcgcgctctga

SEQ ID NO: 100 - DNA, 771 bp, Methylocystis hirsuta
  source 1..771: mol_type = genomic DNA
atgtcggcatcaactgaaacaggctcgccagctggcaagacctggaaatcgaaagaagagtttctcggctgcgtcattctgacggattggatcctgctggtgatcctgttcgccgtgctgctcggctccttccacatccactacatgctgctcgccggcgactgggacttctggatcgacttcaaagatcgacgcatgtggccgacggtgacgccgatcgtcgccatgtgcttcgccgcggccgcgcagtccttcttctggacgaggttccgactgccgatcggcgccacgaccgtcgtgctcgccctgctgattggcgagtggatcaaccgctatgacaacttctggggctggaccttcttcccgatcaatctggtgtttccgtcggcgctcattcccatgggcttctggctcgacatcgtgctgatgatctcgggcagctggctggtcacggcgctcctcggcggccttggctggggcctcctgttctatccgatcaactggcctgtgttggcgcaatatcatcaggccactgaaatcgacggcgtgctgctcacgctcgccgacctcatcggcttcaactacgtccgcaccggcaccccggaatacatccgcatggtcgagcgcggcacgctgcgcaccttcggcaaggacgtcgtgccggtcgccgccttcttctccggcttcatttcgatgctcgtctacttcctatggtggaacatgggcaagtggttctcgacgacgaaatatctcgagatcgaagaggtctag

SEQ ID NO: 101 - DNA, 1290 bp, Methylocystis hirsuta
  source 1..1290: mol_type = genomic DNA
atgaaaagcttgaatttgcagtcgatggccaaatgcgcgacggcccgcgtcgttcgcctttggatcctcggattggccgttgcaggcgcgctaacgaccttgccggcggcgacgcccgccgccgcgcatggcgaacgttcgcagcaggccttcctgcgcatgcgcacgctcaactggtacgacgtcaaatggtcgaagaccgagctcaacgtgaatgatgagatggagctcaccggaaaggtgcatgtgttttccggctggccgcaagccgtcgccaggcccggcgaatcctttctcaacgtcggcgagccggggccggttctcatccgcaagagcgccttcgtcggcgaagtgccggtgccgcgcaccttctcgatggatgtcggcaaggactatgaatacaagatcgtcctgaaagcgcgcaggcaaggccgctatcacgtccatgtccagatcaacgtcaaggacggcggtccgatcgtcggtccagggcagtggatcaccatcaagggcgacatgaaggattttaccaatcccgtgacgctgcttgaaggttcgacgatcgatctcgaaacctatgggatcacctggacctatatctatcacttcttctggatggctgccgccaccgcctggattctctattggttcatgaccaaagggatcatcgtccgatattggcaggtcaaggccggcaagggccgcgagctgatcggtcaagacgaaaagcggatcggcgcgatcacccttgccgcggttctcttggctgtgctcgtcttctatgccgccgccaaccgggaattcccgcgcacgctgccgatgcaggcgggcttgctgaccggcatctcgccgatcgacgagccgcggtcgaccgtcgccgttcagttcaagggcggctcctatacggtgccgggccgtgaattgcggatcagggcgaaggtgaccaacaatggcaaggagccggtccggctcggcgaattcacttccgccggtctgcgcttcctcaatccggaagtctttacggcgcggccggatttccctgactatctgcttgccgaccgcggcctttccgtgagcgattccaacccgatcgcgcccggcgagactcgcgacctggaagtgatcattcaggacgcccgcttcgacatcgaacgcctgtccgatctcgcctatgacaccgatagccagtttggcggactgctgttcttcttcaccccctcgggcgagcgtcagagagctgaaatcggcggcccggttattccgaagttccaggcgggagccacgctctaa

SEQ ID NO: 102 - DNA, 780 bp, Methylocystis hirsuta
  source 1..780: mol_type = genomic DNA
atgatgaatcaaacgacagaaaaagccgtgagcgccgcacaagccggcgagaccgatacgatcgtgaactacaagcccgcctacatcaccatgggcgtgatgctggtgttttacgtcggcatccgaatctatgagcaatatttcggatggaaggccggcctcgattccttcgcgccggaattccagacttactggatgaatttgatgtggaccgagctgccgctcgagttcgtcgccttctgcgccatcggcggctatctctggaagacgcgcgaccgcaacatcgacgccgtcgcgccgcgcgaggagatgcgccgcatcttgactctcatcggctggctcgcggtttacgccttctcggtctactggggcgcctcttacttcaccgaacaggatggcacctggcaccagacggtgattcgcgacaccgacttcacgccaagccacatcctcgagttctacctcagctatccgatctacatcatctgcggctggggcgccttcatgtatgcgcacacgcgcatcccgcagttcgccaagcgaatctcgctcgccttcctgatgttcttcgccgggccgttcatgattttcccgaacatcggcctgaacgagtggggccacactttctggttcatggaagagctgttcaccgctccgctgcactggggcttcgtgttcttcggctggttcgcgctcgccgttttcggcgtcgcgcgccaggtgctcgatcgcgtgatcgaactcagcaaggaatacgagaaggacgctctcgcgctctga

SEQ ID NO: 103 - DNA, 774 bp, Methylocystis hirsuta
  source 1..774: mol_type = genomic DNA
atgagcgtaaccacgcaatcggcagcaggcgcccttgatcgcgccgagccgatcgtcgaattaaagggaatgtggatcgggcttgcggtgctcaacggcttctatctcgtcgtgcgcatctacgagcagatttacggctggcgggccgggctcgattcattcgcgccagagtttcaaacctactggatgagtatcttgtggaccgaaattccgcttgaactcatctcgggcatcggcctcgccggctggctctggaagacgcgcgttcgcgacatgtcgaccgtcacgccgcggcaggaaatgaaggccatcgttgataatgccgcatggctcgtggtctatgcggcggcgatctattggggcgcgtccttcttcacggagcaggacggcacctggcacatgacggtgatacgcgacacggacttcaccccgtcgcacatcatcgagttctacatgagctacccgatctactcgatcatggcgatcggctgctttttccatgccaagacacgaattccctacttcgcgaagggctattcgattgcctatctgatcgtctcgatcggacccttcatgattattccgaacgttggcctgaacgaatggggccacaccttctggttcatggaggaactgttcgttgcgccgctgcattggggcttcgtgttcttcggctggatggcgctcggcgtgttcggagtcgtgatccagctcctgctgaacatccatcgcgttatcggcaaagacggcgtcgagctgctgacggccgcgtaa

SEQ ID NO: 104 - DNA, 753 bp, Methylomicrobium lacus
  source 1..753: mol_type = genomic DNA
atgtcgctacctattaccacacaatcaatcaagccctacgcgggcgaaaaagccaggctcgtcagggcctatgattatctgattctggtcctggccctgttcctttttatcggttcgttccatctgcatttcgcgctgaccgtcggcgactgggatttctgggtggactggaaggaccggcagtggtggcctttggtcacgccgctgatgggcatcacctttccggcggcggtgcaggcggtgttgtgggataaattccggctaccgctcggcgcgaccttgtgcgtcgccgccttgttgatcggcacctgggtcacccgaatctttgcctatcactactggaatttttttccggtcaacatggtgctgccggcgacgatgctgcccggcgcgctggtgctcgatgcgatactgatgttgagcaacagtttcacgatcaccgcgatagtcggcggcagcgccttcgccttgctgttttatccgagcaactggccgattttcgcgctgtttcacatgcctgtcgagtatgcgaatgcccagctgaccctcgccgatttattcggttttcaatacatcagaaccgggatgccggaatatctgcgcatcatcgaacgcggtacgctgcgcacttacggccaatacgcggcgcctctggcggcgttctgttcggcgctgctgtgcacgctgatgtatgccttgtggtggtacatcggcaagtggtttgccaccgtccgttacctgaacaaaatctga

SEQ ID NO: 105 - DNA, 744 bp, Methylomicrobium lacus
  source 1..744: mol_type = genomic DNA
atgagcgcatctcaatcagctgtacgttcacgtgccgaagctgtcaaagtttcacgcacgttcgattggctaatttgtttcactttgttcttcatcatcctcggtggctaccacgttcactttatgttgactggtggtgactgggatttctggactgactggaaagaccgtcgtctgtgggtaactgtactgccaatcgttggtattacattcccagctgctgttcaagcagttctttggtaccgttatcgtctgccattcggcgctatgctggctgttttgggcctgctgttcggcgaatgggttaacagatacttcaacttctggggatggacttatttcccagttaactttgtattcccatcacaattcgttccaggcgctctggtactggacgtaatcctgatgttgtcaaacagcatgcagttgactgctgttctgggtggcttggcttatggtctgttgttctatcctggcaactggcctgtcatcgctccattgcacgtgccagttgaatacaacggcatggtaatgaccctggctgacttgcaaggttaccactatgtaagaactggtactccagaatacattcggatggttgaaaaaggtactttgagaactttcggtaaagacgttgctccagtatctgcgttcttctctggcttcgtaagtatcattatctacttcttgtggcacttcttcggtagatggttcggtagcaccgcatttactcaaagctcttga

SEQ ID NO: 106 - DNA, 1227 bp, Methylomicrobium lacus
  source 1..1227: mol_type = genomic DNA
atgaaaatcaagatgacaaaatgggtttgcgcagtaggattgaccgtgatgggcctctgctcgcccttacaaaccgctcttgcacacggcgaaaaagccctggaaccgttcatccggatgcgcacgattcagtggtatgacgtggcctggtccaagcagcagttcaaggtcaacgaagaagtggcggtgaccggccgttttcatgtcgccgaggactggccggtcagcgtaccgaaacccgaagcgacctttctgaatatctccacacccggaccggtgcttatccgaacagagcgttatttgaacggcaagccatgggtcaattccgtggcgcttcaaccgggcggcgattatgaattcaaggtcgtattaaaaggccgtttgccgggacactatcacattcatccgtttttcaacctgaaggatgccggacaagtgatggggccgggggtctggctcgatatttccggaactgccggcgatttcaacaacgaagtcagaacgctgaacggcgaaatcgtcgatatggaaagcgtcgggctcctcaatggtatcgggtggcacctgttttggggcgtgctggcgacggcctggctgctctggtgggtgcgccggccgctgttcatttcacgttaccggatgctgcaggccggcatggaagaggaactgatcacagccaacgatcgcttgattgccaaggccgtgctggtggcagtgcccgtggctgtgctggccatcaatgcggtcacggtcaatcgctacccgaacgcgattcccctgcaggcgtcgttggaccggatattgccgctgtccgccctggtcaatgcgggccaggtcgatgccgaagcggtcaaggccgaatatgtggtcgcgaagcgggcgatgatttttaccgtcaggatcaataatagcagcgataaaccggttcagatcggcgaattcaataccgcgaacgccagctttatcaatcccgagttgccggccgccgtctcccaggcttcggcaattggagcgactttgagactggatgataatacgccgattcgaccgggcgaagagcgaaccgtcaccgtgacagcgcaagacggcttatgggagaccgaaaaactggatggcctgatcaacgatgcggacagccgcgtcggcggcctgctgtttctttacgctagcgaaggcggcgagcgctacatatcgagtgtgtccgccgccgtgataccgaagttttaa

SEQ ID NO: 107 - DNA, 1245 bp, Methylomicrobium lacus
  source 1..1245: mol_type = genomic DNA
atgaaattaataaaagacaaggtagcaaagttgtcctttgtcgcactgttgctgactgttgcagcagcgatgttctacacaccaactgcgtccgcacacggtgaaaagtctcaagcggctttcatgcgtatgcgtactattcactggtatgatctgaactggtcaaaagaagttgttaaagttaacgacaccatgacaatctctggtaaattcctcgtattcggaggatggccggaaactgttgacaagccagaaagcgctttcttgaacattggtatcccaggcccagtatttatccgtgccggatcttggatcggtggtcaattggttcctcgttcggtttctctggaactgggcgaaacttatgagttcaaagttctgttgaaagcacgccgtccaggtgactggcacgttcacactatgatgaacgttgaaggcggtggtccaatcatcggtcctggtaaatgggtaactgttgaaggttctatgagccaattcaccaacccaatcaccactttgactggtcaaaccattgatctggaaacctacaacctgggcaacacctacttctggcatgctttctggtatgcaattggtgcagcttggatgatctactggatgcgcaaaccaatgttcattccacgtttgatcgctgtagaagctggcaaagcagatactctgatcactgcaactgacaagaaagtcgctctggctttcggtgcagcgacaatcggtatcgttgctttcacaatggctcaaaccaactctgaatacccaattacaactcctctgcaagcaggtttgttgcgtggtatgaagcctcttgatctgccaacaccaactgttagcgttaaaattgacgacgctacctaccgtgtaccaggccgttcaatgaacatgactctgactgtaaccaacaatggcgataccgctgttcgtttgggtgaattcaacactgctggcgttcgtttcttggatgctggcgtcactgaagacaccaccggttacccagatgacttgttggctccagaaggtctgactgttagcgacaacagcccactggctccaggtcaaaccagaactatccaagttaatgcatctgatgcagcttgggaagtatatcgtctggctgacttgatctatgacccagatagccgttttgctggcttgctgttcttccacgatgaaaatggaaacaaacaaaccgttcaaatcgacggtcctttgatcccaaccttcatctaa

SEQ ID NO: 108 - DNA, 774 bp, Methylomicrobium lacus
  source 1..774: mol_type = genomic DNA
atggctacaccatcagaacttatgcccgtggttacaccaaccgggcaccgtctaccctggtatttgcaggatttgccgagatacctgaaaaccttcggcgcgctgaccgtcatgtatatcggcttgcgtctgtatcagggcgcttttgcggtctcggcggggctcgattcgaccgagccggcgttcgagcagtactggatgcggttgttttatattgaactcgcggtgattgcggcggtaatgaccggcctgtgggcctatttgtggctcacgcgcgaccggcagttagacaagctggcgccgaaggaagagattcgccgctatttcacgctgacgatgtggatcagcatttatactttcgcggtgtattgggccggcagttatttcgccgaacaggacaattcctggcatcaggtggcgattcgcgatacgccgttcacggctaaccacatcatcgagttttatttcaattttccgttgtatgtgattctgggcggctgtgcctggctttatgccagaacgcggcttcccttgtatgcgcaaggcatctccctgccattgaccctggcggtgttcggtccgttcatgatcctggtcagcgtcggcttcaacgaatggggacataccttctggtttcgcgaagaattcttcgccgcgccgattcactggggcttcgtgatcggcgtctggtttgccctgggcgtgggcggcattcttttacaaggcgtcactcggttaatcgaattattggataagatcgaagatgccgaataa

SEQ ID NO: 109 - DNA, 753 bp, Methylomicrobium lacus
  source 1..753: mol_type = genomic DNA
atggcagctacaactgaatcagtgaaagctgatgctgcggaagcaccgctgttaaataaaagaaatctgttcctgggcgttgcgatatacaacgtattctatgcttgggttcgttggtacgaaggcgtttacggctggtccgctggtcttgactctttcgcgcctgagttcgaaacatactggatgaacttcctgtacatcgagttcgtattagaagtatcaaccgcaggtatcctgtggggctacatctggaagactcgtgaccgcaaagtgatgtcaatcactccaagagaagaactgagaagacacttcactcactggacatggttggtttgctactctgtagctatttacttcggcgctagctacttcactgagcaagatggtacatggcatcaaactatcgttcgtgataccgacttcactccaagtcatatcatcgaattctacttgagctacccaatctatatcatcactggtggcgcttctttcctgtatgctaaaaccagactgcctacatatcaacaaggcttgtccttgcaatatatggtgtcagttgttggtcctttcatgattctgccaaacgttggtctgaatgagtggggtcacactttctggtttatggaagagttgttcgttgctcctctgcactatggcttcgtattcttcggatgggctgctctgggtgcactgggtgttctgaacatcgaatgccaagcaattgctaaattgctgaaaaaagacttggcttaa

SEQ ID NO: 110 - DNA, 753 bp, Methylosarcina fibrata
  source 1..753: mol_type = genomic DNA
atggctgcaaatcttaccaaacaaatttttcgaccctataccggagaaaaggcgcgaatcacccgcgcttacgattatctgatcctggtattggcactgtttttatttatcggttcctttcatctgcattttgccttgaccgtcggggattgggacttctggatagactggaaagaccggcaatggtggccgctggttacgccgctgatgggaattacctttccggcggcggtgcaggcggtgatgtgggaaaaatttagactgccgttcggggcaacattgtgcgtggcggcattgttgataggaacctggattacccgggtgtttgcctatcattactggaattactttcccatcaatatggtgctgccggcaacgatgttgccaagcgcattggttttggacgttattctgatgctaagcaacagcctgaccatcacggctatttttggcggaggtgcatttgccttattgttttatcccagcaactggccaattttcgccatgttccacgagcctatcgaatccggcaacggacaattgacggtagccgatttgttcggattccagtatatccgcaccggcatgccggaatatttacggatcatcgaacggggtactttgcgtacttacggtcaatatgcggcaccgttagcggctttttgttcggctttgttatgttccttgatgtataccttgtggtggtatatcgggaagtggttcgcaacggttcgttatctcgccaaaatctga

SEQ ID NO: 111 - DNA, 744 bp, Methylosarcina fibrata
  source 1..744: mol_type = genomic DNA
atgtcaactactagtttatccatcggttttgataaagaaaccgccacagtctccaggatttatgattatctaatcctggtgttagccggatttttatatattggctcttatcacctgcattttatgctaacggtgggtgactgggatttttggctcgactggaaagaccggcagtactggccactggtgacgcctgtgctaatgattgcctggcccgcagccgtgcaatcggtattgtggacccattttcgcttgccaatgggtgcaactttatcgatagtgggtttaatgctgggcatgtggatcacgcgcgtgaccgcctatcaagtttggaccaattacccacttaattttgtgctaccggccaccatgatcccgagcgccttagttctggatgccattttaatgttatcccccagttttctagtgaccgccatcataggaggggcttcttttgcgcttttattctacccaactaactggccaatttttgccatgttccatgttcccattgaatggcaaggtggattgcaaaccatcgccgatcaatttggttttgaatatttacgggcgggcatgccggaatatctgcgcatcatagaacgtggtacgttacgaacctatggtcaaatggctgctccgttgtccgccttttttgcggcactgctctgtatcctggtttatgccgcaggctggtatatcggtaaagcctttgccactgtccgttatctgaaacgtgtatag

SEQ ID NO: 112 - DNA, 744 bp, Methylosarcina fibrata
  source 1..744: mol_type = genomic DNA
atgagcgcatctcaatcagctgtacgttcacgcgcggaagcagtcaaagtttctcgcacgttcgattggctaatctgttttgctttgttcttcactattctcggtggctatcacgttcacttcatgttgactggtggtgactgggatttctggacagactggaaagaccgtcgtctttgggtaaccgtactgcctatcgttggtattacattcccagcagctgttcaagcagttttgtggtggcgttaccgtctgccattcggtgcagttctgtccgttttgggtctgctgttcggcgaatgggttaacagatacttcaacttctggggatggacttatttcccagttaactttgtattcccatcacaattcgttccaggcgctatcgttctggacgtcattctgatgttgtctaacagcatgcagttaacagctgtaatcggcggtttggcttatggtttgttgttctatcctggcaactggcctgtcatcgctccattacacgttcctgtagaatacaacggtatggtaatgactcttgctgacttgcaaggttaccactatgtaagaactggtacacctgagtacatccggatggttgaaaaaggtacactgagaactttcggtaaagacgttgctcctgtatctgcgttcttctctggtttcgtgagtatcattatttacttcttgtggcatttcttcggaagatggttcggtcaacaaactttcacttcgcaaacctaa

SEQ ID NO: 113 - DNA, 1239 bp, Methylosarcina fibrata
  source 1..1239: mol_type = genomic DNA
atgaaaaacttattaataaaaagcgtattctcgctgagtctcgttctttcactgtatgcgctgaacattcaaaccgtgatggcgcatggcgaaaaggcgttagaaccgtttatcagaatgcgcacgattcaatggtatgacgtgcaatggtccagacaaaaatttgcagtcaatgaagaaatcagtgttagcggcaaatttcatgtggccgaagattggcctgtcagtgtacctaagccggatgcagccttcttgaacatatcgacgccggggcctgtattgatcagaactgaacgctatttgaacgggaaaccctatgtaaattcggtagccctacagccgggcggcgattacagcttcaaagttgtccttaagggacgtttgccgggccgttatcacatccatcccttttttaatctgaaagatgccggtcaggtgatgggtcccggggtatggctggatgtgacaggcgatgcgtccgatttcagcaacgaagccaaaaccattaatggcgaagtcatcaatatggaaacctatggattgggcaatggcatattctggcatatcttttggggagcgttcgcttcggcctggctgctctggtgggtgcggcggccgttatttattccccgctaccggatgctgcaagcaggcctggaagatgaactggtcacccggcaggataaacatatcggtaaggccattttgattggcgtaccgttattcgtactggctgctaactggatggcgatcgatcagcatccaaaagccatccctttgcaggctgcactcgatcagatactgccgctgtcagcgcaggtcaatgcaggcgtggtcgatgtcgacaccttaaaagccgaatatcggataccggaacgtgcgatgacttttcttgtaaaagtcaataatagaagcgataagcctgttcaggtgggtgagttagccgttgccaatgtccgcttcctcaacaaagaatttgctcagcccggtcaggagactaccgacaggatggtagctccggatggattgactctatctgatgccactcccatccagcccggcgaagaacgcacgatgacgattatcgctaaagatgcgttatgggaatctgaaaggttagatgggctgatacgggattccgatagccggattggaggtctgctgtttttctacgatgggttaataggcgagcgttttatctccagtataaccgccgctgtgatcccgaaatttgattaa

SEQ ID NO: 114 - DNA, 1245 bp, Methylosarcina fibrata
  source 1..1245: mol_type = genomic DNA
atgaaattaataaaagacaaggtagcaaagttgtcctttgtcgcactgctgttgactgttacggcagcgatgttctacacgccaactgcatcagctcatggtgaaaaatcgcaagcggcattcatgcgtatgcgtacaattcactggtacgatcttaactggtcaaaagaagtgttggccgttaacgatacaatgacgatttccggtaaattcctggtattcgcaggatggcctgaaactgttgataaaccagaaagcgcattcttgaacattggtattcctggaccagtatttatccgtgctggatcttggatcggtggtcaattggttcctcgttccgtttctttggaattgggcgaaacttatgagtttaaagttctgttgaaagcacgtcgtccaggtgactggcacgttcacactatgatgaacgttgaaggcggtggtccaatcatcggaccaggaaaatgggtaaccatcactggttcaatgagctcgttcaccaacccaatcactacgttgactggtcaaacaattgatctggaaacttacaatctgagcaacacctacttctggcatgctttctggtatgctatcggtatcgcttggatggtatattggatgcgtaaaccaatgttcgttcctcgtatgatcgctgttgacgctggcaaagctgattctctgatcactggaaccgatagaaaagttgctctggcattcgctgccggttcaattgctatcgttgcattcacaatgcaaagcaccaactcgcaatatcctgtaacaactccgttgcaagcaggtttgttgcgtggcatgaagccaattgaaatgccaacaccaactgttagcgtgaaagtagatgatgctacctatcgtgtaccaggccgttcaatgaacatgactctgactgtaaccaataacggtgattcagcagtccgtttaggtgaattcaacaccgctggcgttcgtttcttggatgctaacgtaactgaagacaccaccggctacccagatgacttgttggctccagaaggtttgactgttagcgataacagccctctggctccaggccaatcaagaaccatccaagtaaccgcatctgatgcagcttgggaagtttatcgtttggctgacttgatctacgatccagacagccgcttcgctggtttgttgttcttccacgacgaaagtggcaacaagcaagttgtaacgatcgatgcaccattaatcccaactttcatctaa

SEQ ID NO: 115 - DNA, 774 bp, Methylosarcina fibrata
  source 1..774: mol_type = genomic DNA
atggctacaacatccgaacaactatcttcatcggtagaaaatgacaacaaagtgccctggtatttgctggatttaccgaggtatctgaagggctttggcgtactgacggtgatctatatcgggctacgcctatatcaaggcgcttttgccgtttcgtcgggactcgactcgaccgaaccggctttcgaacggtactggatgcggttgttttacattgaattaggcgtaatcgccgttttcgtgaccggaatttggggctatttatggttttcccgggatcgtcatctggacaccctgtcaccgcgggaagaaatccgtcgttattttaccctgaccatgtggatcagcatctacacctttgccgtctattgggccggcagttacttcgccgaacaggacaattcctggcatcaggtcgccattcgggatacgccttttactgccaatcatatcattgagttttatttcaatttcccgctttatgtgattttaggcggctccgcctggttatacgccagaacacgtctaccgctttatgcaaaagggatttcgctgcccctgacgctggcggtattcgggccgttcatgattctggtcagcgttggctttaacgagtggggacataccttctggtttagagaggaattcttcgctgcgcctatccactgggggtttgtgattggcgtctggtttgcattaggggtaggcgggatactactgcaaggcgtcacgcgaatgattgaattacttgatgaactcgaagaaaaaccctga

SEQ ID NO: 116 - DNA, 789 bp, Methylosarcina fibrata
  source 1..789: mol_type = genomic DNA
atggttactgcacctgataaaatcatggcaccatccagcactgaaacctacggtagccagtggtatctgaaagagtttaaacgatacgctgtgactttcttggcgattttgttcatgtatgtatcgatcaccatctatcaatatacctttggtgcggccacgggcctagactcaaccgatcctctatttgaggaatactggatgaatctattttatatcgaggtcatttttcaactcatcttgatcactgcactaggctattatttttggaaaaatcgtgatctgcatcttgatcaactgtctccacgcgaggaattgcgccgatatttgaacctgaccatgtggatcagtatttataccttctctgtttattgggcaggtagttattttgccgaacaggataatgcctggcatcaggctgccattcgcgacaacatttttaccgccaaccatgtcgtggaattttatctgtgctttccgttttataccatcatgggtatttcatcatggctatatgcgcgtacccaattaccgttatttgctaaaaacatttcgctgccgttaaccttggctgtggcgggccccttcatgatttttgtcgctgttggctttaacgaatggggacatacattttggtttagagaagaattgtttggtgcttatatgcattatgccttcgtcatcggagtatggtttgcacttgccgttggaggcattttgttgcaacaagtcatgcgtatgtccgagctgattgaactcattaatcaagataattcagcacatccttaa

SEQ ID NO: 117 - DNA, 753 bp, Methylosarcina fibrata
  source 1..753: mol_type = genomic DNA
atggcagctacaactgaatcagtgaaagctgatgctgcggaagcaccgctgttaaataaaagaaatctgttcctgggcgtagcactgtacaacgtgttctatgcatgggttcgctggtatgaaggcgtctatggctggtctgctggtctggactcatttgcaccagagtttgaaacatactggatgaacttcctgtacatcgagttcgttctggaagtatcgactgcaggtatcctgtggggctacatctggaagactcgtgaccgtaaagttatgtcaatcactccaagagaagagttgagaagacacttcacacactggatctggctggtttgctacagcgttgcaatttacttcggcgctagctacttcacagagcaagatggtacatggcatcaaacgatcgttcgcgataccgacttcactccaagtcacattatcgagttctatctgagctatcctatctacatcatcactggtggtgcatcttacctttacgctaaaaccagactgccaacctatcagcaaggtcactccattgcttacatggttgccgttgtaggtcctttcatgattctgccaaacgttggtctgaacgaatggggtcacactttctggtttatggaagagttgttcgttgctcctttacactacggcttcgtattcttcggatgggctgctctgggtgtattgggtgtattgaacatcgaagtttcagcactttcaaaacttctgaaaaaagatctggcttaa

SEQ ID NO: 118 - DNA, 1483 bp, Methylocystis hirsuta
  source 1..1483: mol_type = genomic DNA
cttgagagtttgatcctggctcagaacgaacgctggcggcaggcctaacacatgcaagtcgaacgccctagcaatagggagtggcagacgggtgagtaacgcgtgggaacgtacccttcggtctggaataacccagggaaacttgggctaataccggatacgtgcgagagcagaaagatttatcgccgaaggatcggcccgcgtccgattagctagttggtgaggtaaaagctcaccaaggcgacgatcggtagctggtctgagaggatgatcagccacactgggactgagacacggcccagactcctacgggaggcagcagtggggaatattggacaatgggcgaaagcctgatccagccatgccgcgtgagtgatgaaggccctagggttgtaaagctctttcgccagggacgataatgacggtacctggataagaagccccggctaacttcgtgccagcagccgcggtaatacgaagggggctagcgttgttcggaatcactgggcgtaaagcgcacgtaggcggatctttaagtcaggggtgaaatcccgaggctcaacctcggaactgcctttgatactggaggtctcgagtccgggagaggtgagtggaactgcgagtgtagaggtgaaattcgtagatattcgcaagaacaccagtggcgaaggcggctcactggcccggtactgacgctgaggtgcgaaagcgtggggagcaaacaggattagataccctggtagtccacgccgtaaactatggatgctagccgttgggcagcttgctgttcagtggcgcagctaacgctttaagcatcccgcctggggagtacggtcgcaagattaaaactcaaaggaattgacgggggcccgcacaagcggtggagcatgtggtttaattcgaagcaacgcgcagaaccttaccagcttttgacatgcccggtatgatcgccagagatggctttcttcccgcaaggggccggtgcacaggtgctgcatggctgtcgtcagctcgtgtcgtgagatgttgggttaagtcccgcaacgagcgcaaccctcgcccttagttgccatcattaagttgggcactctagggggactgccggtgataagccgcgaggaaggtggggatgacgtcaagtcctcatggcccttacaggctgggctacacacgtgctacaatggcggtgacaatgggatgcggaagggcgacctcgagcaaatctcaaaaagccgtctcagttcggattgcactctgcaactcgagtgcatgaaggtggaatcgctagtaatcgcagatcagcacgctgcggtgaatacgttcccgggccttgtacacaccgcccgtcacaccatgggagttggttttacccgaaggcgtttcgccaaccgcaaggaggcaggcgaccacggtagggtcagcgactggggtgaagtcgtaacaaggtagccgtaggggaacctgcggctggatcacctccttt

SEQ ID NO: 119 - DNA, 1440 bp, Methylomicrobium lacus
  source 1..1440: mol_type = genomic DNA
tgaagagtttgatcatggctcagattgaacgctggcggtatgcttaacacatgcaagtcgaacggtaacaggccttcgggcgctgacgagtggcggacgggtgcgtaacgcgtaggaatctgcctgttagtgggggacaacgtggggaaactcacgctaataccgcatacgccctacgggggaaagcgggggaccttcgggcctcgcgctaacagatgagcctgcgtgggattagctagttggtagggtaaaggcctaccaaggcgacgatccctagctggtctgagaggacgatcagccacactgggactgagacacggcccagactcctacgggaggcagcagtggggaatattggacaatgggcgcaagcctgatccagcaataccgcgtgtgtgaagaaggcctgagggttgtaaagcacttttaatcgggaggaacacctgtcagcgaatacctggcagtatgacattaccgatagaaaaagcaccggctaactccgtgccagcagccgcggtaatacggagggtgcaagcgttaatcggaattactgggcgtaaagcgtgcgtaggcggctgtttaagtcagatgtgaaagccccgggcttaacctgggaactgcatttgatactgggcagctagagttgagtagaggagaggggaatttcaggtgtagcggtgaaatgcgtagagatctgaaggaacaccagtggcgaaggcgcctctctggactcaaactgacgctgaggtacgaaagcgtgggtagcaaacaggattagataccctggtagtccacgccgtaaacgatgtcaactagccgttggtcttattcgtaagattagtggcgtagctaacgcgataagttgaccgcctggggagtacggccgcaaggttaaaactcaaatgaattgacgggggcccgcacaagcggtggagcatgtggtttaattcgatgcaacgcgaagaaccttacctacccttgacatccagagaatctgttagagatagtggagtgccttcgggaactctgagacaggtgctgcatggctgtcgtcagctcgtgtcgtgagatgttgggttaagtcccgtaacgagcgcaacccttatccttagttgccagcgggtcatgccgggaactctagggagactgccggtgataaaccggaggaaggtggggacgacgtcaagtcatcatggcccttatgggtagggctacacacgtgctacaatggccggtacagagggctgcgaacttgcgaaagtaagccaatcccacaaagccggtcctagtccggattggagtctgcaactcgactccatgaagtcggaatcgctagtaatcgcgaatcagaatgtcgcggtgaatacgttcccgggccttgtacacaccgcccgtcacaccatgggagtgggttgcaaaagaagtaggtagtctaacctt

SEQ ID NO: 120 - DNA, 1530 bp, Methylosarcina fibrata
  source 1..1530: mol_type = genomic DNA
tgaagagtttgatcatggctcagattgaacgctggcggtatgcttaacacatgcaagtcgaacggtaacaggccttcgggcgctgacgagtggcggacgggtgcgtaacgcgtaggaatctgcctggtagtgggggacaacttggggaaactcaagctaataccgcatacgccctacgggggaaagcgggggatcttcggacctcgcgctatcagatgagcctgcgttggattagctagttggtagggtaaaggcctaccaaggcgacgatccatagctggtctgagaggacgatcagccacactgggactgagacacggcccagactcctacgggaggcagcagtggggaatattggacaatgggcgcaagcctgatccagcaataccgcgtgtgtgaagaaggcctgagggttgtaaagcacttttaatcgggaggaaaacctgtcggccaatacccgacagcttgacattaccgatagaaaaagcaccggctaactccgtgccagcagccgcggtaatacggagggtgcaagcgttaatcggaattactgggcgtaaagcgtgcgtaggcggccttttaagtcagatgtgaaagccccgggcttaacctgggaactgcatttgatactgggaggctagagtttaatagaggggagtggaatttcaggtgtagcggtgaaatgcgtagagatctgaaggaacaccagtggcgaaggcgactccctggattaaaactgacgctgaggtacgaaagcgtgggtagcaaacaggattagataccctggtagtccacgccgtaaacgatgtcaactagccgttggtcttatttataagattagtggcgcagctaacgcattaagttgaccgcctggggagtacggccgcaaggttaaaactcaaatgaattgacgggggcccgcacaagcggtggagcatgtggtttaattcgatgcaacgcgaagaaccttacctacccttgacatccagagaatctgttagagatagcggagtgccttcgggaactctgagacaggtgctgcatggctgtcgtcagctcgtgtcgtgagatgttgggttaagtcccgtaacgagcgcaacccttatccttagttgccaacaggtcatgctgggaactctagggagactgccggtgataaaccggaggaaggtggggacgacgtcaagtcatcatggcccttatgggtagggctacacacgtgctacaatggccggtacagagggcagccagctcgcgagagtcagcaaatcccacaaagccggtcctagtccggattggagtctgcaactcgactccatgaagtcggaatcgctagtaatcgcgaatcagaatgtcgcggtgaatacgttcccgggccttgtacacaccgcccgtcacaccatgggagtgggttgcaaaagaagtaggtagtctaaccttcgggagggcgcttaccactttgtgattcatgactggggtgaagtcgtaacaaggtagccctaggggaacctggggctggatcacctcctt

SEQ ID NO: 121 - DNA, 1471 bp, Methylobacterium radiotolerans
  source 1..1471: mol_type = genomic DNA
gagtttgatcctggctcagagcgaacgctggcggcaggcttaacacatgcaagtcgagcgggcctttcggggtcagcggcggacgggtgagtaacgcgtgggaacgtgccttctggttcggaataaccctgggaaactagggctaataccggatacgcccttttggggaaaggtttactgccggaagatcggcccgcgtctgattagctagttggtggggtaacggcctaccaaggcgacgatcagtagctggtctgagaggatgatcagccacactgggactgagacacggcccagactcctacgggaggcagcagtggggaatattggacaatgggcgcaagcctgatccagccatgccgcgtgagtgatgaaggccttagggttgtaaagctcttttatccgggacgataatgacggtaccggaggaataagccccggctaacttcgtgccagcagccgcggtaatacgaagggggctagcgttgctcggaatcactgggcgtaaagggcgcgtaggcggcgttttaagtcgggggtgaaagcctgtggctcaaccacagaatggccttcgatactgggacgcttgagtatggtagaggttggtggaactgcgagtgtagaggtgaaattcgtagatattcgcaagaacaccggtggcgaaggcggccaactggaccattactgacgctgaggcgcgaaagcgtggggagcaaacaggattagataccctggtagtccacgccgtaaacgatgaatgccagctgttggggtgcttgcaccgcagtagcgcagctaacgctttgagcattccgcctggggagtacggtcgcaagattaaaactcaaaggaattgacgggggcccgcacaagcggtggagcatgtggtttaattcgaagcaacgcgcagaaccttaccatcctttgacatggcgtgttacccagagagatttggggtccacttcggtggcgcgcacacaggtgctgcatggctgtcgtcagctcgtgtcgtgagatgttgggttaagtcccgcaacgagcgcaacccacgtccttagttgccatcattcagttgggcactctagggagactgccggtgataagccgcgaggaaggtgtggatgacgtcaagtcctcatggcccttacgggatgggctacacacgtgctacaatggcggtgacagtgggacgcgaaggagcgatctggagcaaatccccaaaagccgtctcagttcggattgcactctgcaactcgagtgcatgaaggcggaatcgctagtaatcgtggatcagcatgccacggtgaatacgttcccgggccttgtacacaccgcccgtcacaccatgggagttggtcttacccgacggcgctgcgccaaccgcaaggaggcaggcgaccacggtagggtcagcgactggggtgaagtcgtaacaaggtagccgtaggggaacctgcggctggatcacctc

SEQ ID NO: 122 - DNA, 1220 bp, Methylorubrum populi
  source 1..1220: mol_type = genomic DNA
aaaggtttactgccgaaggatcggcccgcgtctgattagcttgttggtggggtaacggcctaccaaggcgacgatcagtagctggtctgagaggatgatcagccacactgggactgagacacggcccagactcctacgggaggcagcagtggggaatattggacaatgggcgcaagcctgatccagccatgccgcgtgagtgatgaaggccttagggttgtaaagctcttttgtccgggacgataatgacggtaccggaagaataagccccggctaacttcgtgccagcagccgcggtaatacgaagggggctagcgttgctcggaatcactgggcgtaaagggcgcgtaggcggccgattaagtcgggggtgaaagcctgtggctcaaccacagaattgccttcgatactggttggcttgagaccggaagaggacagcggaactgcgagtgtagaggtgaaattcgtagatattcgcaagaacaccagtggcgaaggcggctgtctggtccggttctgacgctgaggcgcgaaagcgtggggagcaaacaggattagataccctggtagtccacgccgtaaacgatgaatgccagccgttggtctgcttgcaggtcagtggcgccgctaacgcattaagcattccgcctggggagtacggtcgcaagattaaaactcaaaggaattgacgggggcccgcacaagcggtggagcatgtggtttaattcgaagcaacgcgcagaaccttaccatcccttgacatggcatgttacctcgagagatcggggatcctcttcggaggcgtgcacacaggtgctgcatggctgtcgtcagctcgtgtcgtgagatgttgggttaagtcccgcaacgagcgcaacccacgtccttagttgccatcattcagttgggcactctagggagactgccggtgataagccgcgaggaaggtgtggatgacgtcaagtcctcatggcccttacgggatgggctacacacgtgctacaatggcggtgacagtgggacgcgaaaccgcgaggttgagcaaatccccaaaagccgtctcagttcggattgcactctgcaactcgggtgcatgaaggcggaatcgctagtaatcgtggatcagcacgccacggtgaatacgttcccgggccttgtacacaccgcccgtcacaccatgggagttggtcttacccgacggcgctgcgccaaccgcaagg

SEQ ID NO: 123 - protein, 212 aa, Methylobacterium populi
MNPLQTILYARVSTADQTSAHQRTQAEAAGFKIDAVIADDGVSGVSTILAERPQGKRLFDMLRRGDVLVVRWVDRLGRSYADVTETIREFMRRGVIVRTVINNMTFDGATTDPVQMAVRDALIGFMAATAQAQGEATREAQKAGIAHARDREEAYRGRKPSFTREQFQTVRDRSELVGVSEIARETGLSRQTVYRILQSPADAEAALGRWEA

SEQ ID NO: 124 - protein, 168 aa, Methylobacterium radiotolerans
MPVMTAREFADTIAVPTVEDFMRDPCQRRGYLACLAAYHLGDYLKPTGAWAPKAVHQAMETELGAPFLALQRMANAVKHREKAEGGIVLMQSGSDQERPPAGFGQASADNPFRFEDYGGRWIEDGGRRFDMLDLCLIVLRAYAKLHPDELSGSLIEGIRYNTATYPRI

SEQ ID NO: 125 - protein, 66 aa, Methylobacterium radiotolerans
MLSFQDVADAGPEERDAIMRALAEEMMEVSGQRRGGRGFVRAERPRDLARVLGRDRRERRPQRQAG

SEQ ID NO: 126 - protein, 54 aa, Methylobacterium radiotolerans
MPVGIGIGRGDPLRPAVTRTARFSGPEGFHPGALWLAAAAPLLATLLLLVRLAA

SEQ ID NO: 127 - protein, 210 aa, Methylobacterium radiotolerans
MQTVLYARVSTAEQTIAHQRKQAEAAGFRIDEVVADEGVSGVSTVLAERPQGRRLFDMLRAGDVLVVRWVDRLGRNYADVTATIREFMRRGVVVRTVINAMTFDGTTTDPVQMAVRDALIGFMAATAQAQAEVTRDAQRAGIDYAKGREDAYLGRKPSYSAGQLADVVAGLDRGDTVAAIAKATRLTRQTVYRIKNNRAEAEAALARWAA

SEQ ID NO: 128 - protein, 54 aa, Methylobacterium radiotolerans; identical to SEQ ID NO: 30